(19) United States
(12) Patent Application Publication
(10) Pub. No.: US 2015/0133431 A1
(43) Pub. Date: May 14, 2015

(54) GGA DERIVATIVES

(71) Applicant: COYOTE PHARMACEUTICALS, INC., Menlo Park, CA (US)

(72) Inventor: Gary C. Look, Santa Clara, CA (US)

(73) Assignee: COYOTE PHARMACEUTICALS, INC., Menlo Park, CA (US)

(21) Appl. No.: 14/328,616

(22) Filed: Jul. 10, 2014

Related U.S. Application Data
(60) Provisional application No. 61/845,302, filed on Jul. 11, 2013.

(51) Int. Cl. C07C 49/203; C07D 271/06; C07D 401/06; C07D 263/32; C07D 263/34; C07D 271/10; C07D 263/08; C07D 263/30

(52) U.S. Cl. CPC C07C 49/203; C07D 263/08; C07D 271/06; C07D 263/30; C07D 263/32; C07D 263/34; C07D 271/10; C07D 401/06
USPC 514/217.11; 548/239; 548/228; 548/233; 548/131; 548/132; 548/133; 548/235; 548/311.1; 546/210; 548/186; 548/183; 548/184; 546/209; 548/231; 548/531; 548/541; 546/242; 540/604; 544/178; 544/162; 544/170; 568/417; 514/374; 514/361; 514/376; 514/397; 514/326; 514/369

(57) ABSTRACT

This invention relates to geranylgeranyl acetone (GGA) derivatives, pharmaceutical compositions comprising GGA derivatives and the use of GGA derivatives.

### REFERENCE TO RELATED APPLICATION

This application claims priority under 35 U.S.C. section 119(e)(1) to U.S. provisional application No. 61/845,302, filed Jul. 11, 2013, which is incorporated herein in its entirety by reference.

### FIELD OF THE INVENTION

This invention relates generally to GGA derivatives, compositions comprising and methods for using the same.

### STATE OF THE ART

Geranylgeranyl acetone is an acyclic isoprenoid compound with a retinoid skeleton that has been shown to induce expression of heat shock proteins in various tissue types. GGA is a known anti-ulcer drug used commercially and in clinical situations.

GGA has also been shown to exert cytoprotective effects on a variety of organs, such as the eye, brain, and heart (See for example Ishii Y., et al., Invest Ophthalmol V is Sci 2003; 44:1982-92; Tanito M, et al., J Neurosci 2005; 25:2396-404; Fujiki M, et al., J Neurotrauma 2006; 23:1164-78; Yasuda H, et al., Brain Res 2005; 1032:176-82; Ooie T, et al., Circulation 2001; 104:1837-43; and Suzuki S, et al., Kidney Int 2005; 67:2210-20). The effects and cytoprotective benefits of GGA in these settings is less understood as is the relationship of isomers of GGA to these cytoprotective benefits. There is a need for GGA derivatives for use in these and other therapies.

### SUMMARY OF THE INVENTION

In various aspects, provided herein are GGA derivatives, such as those of Formulas (I) and (VIII)-(XXII), and sub-formulas thereof, compositions, preferably pharmaceutical formulations, thereof, processes of their syntheses, and their use, wherein Formulas (I) and (VIII)-(XXII) are shown below:

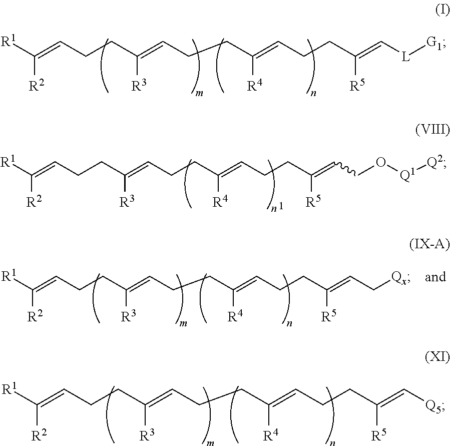

or pharmaceutically acceptable salt thereof; wherein Qx is Q₃, Q₄, or Q₆, and all the variables for Formulas (I), (VIII), (IX), and (XI) are as defined herein.

In one aspect, this invention provides a pharmaceutical composition comprising an effective amount of a GGA derivatives, such as those of Formulas (I), (VIII), (IX), and (XI), and sub-formulas thereof, and optionally at least one pharmaceutical excipient. In some non-limiting embodiments, the compositions are suitably formulated for oral administration, such as an enteric coated oral formulation, intranasal administration, sublingual administration, topical ocular administration, parenteral administration through the ocular surface of a patient, etc.

In another aspect, this invention provides a method for treating a disease or disorder. Non-limiting disease or disorders include osteoporosis, a neural disorder or disease (e.g., for inhibiting neural death, increasing neural activity or treating paralysis); ulcers; chronic liver disease (CLD), inflammatory bowel disease (IBD), coronary heart disease (CHD), cardiac ischemia, liver injury disorder, acute liver failure, myocardial infarcation; an ocular neural disease, optic nerve damage, glaucoma, etc. In other embodiments, the treatment relates to providing cytoprotective effects on a variety of organs, such as the eye, brain, and heart; inducing expression of a heat shock protein, in ocular tissue, and inhibiting apoptosis of a retinal ganglion cell. The compounds and compositions provided herein are administered for such treatment.

In one embodiment, the GGA derivative used according to this invention is 5-trans GGA derivative or substantially pure 5-trans GGA derivative which is optionally free of cis GGA derivative or is essentially free of cis GGA derivative.

### DETAILED DESCRIPTION

It is to be understood that this invention is not limited to particular embodiments described. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”, “an”, and “the” include plural referents unless the context clearly dictates otherwise. Thus, for example, reference to “an excipient” includes a plurality of excipients.

### 1. DEFINITIONS

Unless defined otherwise, all technical and scientific terms used herein have the same meaning as commonly understood by one of ordinary skill in the art to which this invention belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, the term “treatment” or “treating” means any treatment of a disease or condition in a patient, including one or more of:
preventing or protecting against the disease or condition, that is, causing the clinical symptoms not to develop, for example, in a subject at risk of suffering from such a disease or condition, thereby substantially averting onset of the disease or condition;
inhibiting the disease or condition, that is, arresting or suppressing the development of clinical symptoms; and/or
relieving the disease or condition that is, causing the regression of clinical symptoms.

An effective amount of GGA derivative is the amount of GGA derivative required to produce a protective effect in vitro or in vivo.

Routes of administration refers to the method for administering GGA derivative to a mammal. Administration can be achieved by a variety of methods. These include but are not limited to subcutaneous, intravenous, transdermal, sublingual, or intraperitoneal injection or oral administration.

The term “about” when used before a numerical designation, e.g., temperature, time, amount, and concentration, including range, indicates approximations which may vary by (+) or (−) 10%, 5%, or 1%.

The term “halogenating” is defined as converting a hydroxy group to a halo group. The term “halo” or “halo group” refers to fluoro, chloro, bromo and iodo.

The term “stereoselectively” is defined as providing over 90% of the E isomer for the newly formed double bond.

“Geometrical isomer” or “geometrical isomers” refer to compounds that differ in the geometry of one or more olefinic centers. “E” or “(E)” refers to the trans orientation and “Z” or “(Z)” refers to the cis orientation.

Geranylgeranyl acetone (GGA) refers to a compound of the formula:

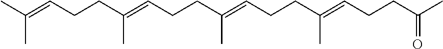 [SMILES: CC(=O)CC/C=C(\C)CC/C=C(\C)CC/C=C(\C)CCC=C(C)C]

wherein compositions comprising the compound are mixtures of geometrical isomers of the compound.

The 5-trans isomer of geranylgeranyl acetone refers to a compound of the formula VI:

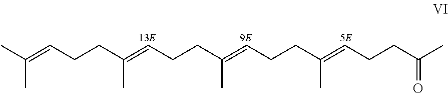

wherein the number 5 carbon atom is in the 5-trans (5E) configuration.

The 5-cis isomer of geranylgeranyl acetone refers to a compound of the formula VII:

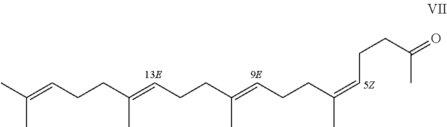

wherein the number 5 carbon atom is in the 5-cis (5Z) configuration.

“Trans” in the context of GGA derivatives refer to the GGA scaffold as illustrated below:

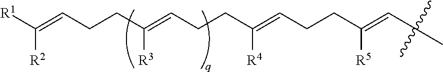

wherein R¹-R⁵ is defined herein and q is 0-2. As shown, each double bond is in a trans or E configuration. In contrast, a cis form of GGA or a GGA derivative will contain one or more of these bonds in a cis or Z configuration.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques.

As used herein, Cₘ-Cₙ, such as C₁-C₁₀, C₁-C₆, or C₁-C₄ when used before a group refers to that group containing m to n carbon atoms.

The term “about” when used before a numerical designation, e.g., temperature, time, amount, and concentration, including range, indicates approximations which may vary by (+) or (−) 10%, 5% or 1%.

The term “alkoxy” refers to —O-alkyl.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). In some embodiments, the term “alkyl” refers to substituted or unsubstituted, straight chain or branched alkyl groups with C₁-C₁₂, C₁-C₆ and preferably C₁-C₄ carbon atoms.

The terms “alkylene” alone or as part of another substituent means a divalent radical derived from an C₁-C₆ alkyl group as described herein, optionally substituted with 1-3 C₁-C₆ alkyl groups, as exemplified by —CH₂—, —CH₂CH₂—, and —CH₂CH₂CH(CH₃)—. For alkylene linking groups, no orientation of the linking group is implied.

The term “amide” means —CONR₂, where R is hydrogen or C₁-C₆ alkyl group as described herein, optionally substituted with 1-3 C₁-C₆ alkyl groups.

The term “ester” means —COOR, where R is C₁-C₆ alkyl group as described herein, optionally substituted with 1-3 C₁-C₆ alkyl groups.

The term “aryl” refers to a monovalent, aromatic mono- or bicyclic ring having 6-10 ring carbon atoms. Examples of aryl include phenyl and naphthyl. The condensed ring may or may not be aromatic provided that the point of attachment is at an aromatic carbon atom. For example, and without limitation, the following is an aryl group:

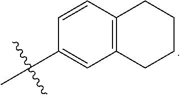 [SMILES: *c1ccc2c(c1)CCCC2]

In some embodiments, the term “aryl” refers to a 6 to 10 membered, preferably 6 membered aryl group. An aryl group may be substituted with 1-5, preferably 1-3, halo, alkyl, and/or —O-alkyl groups.

The term “—CO₂H ester” refers to an ester formed between the —CO₂H group and an alcohol, preferably an aliphatic alcohol. A preferred example included —CO₂R^{E}, wherein R^{E} is alkyl or aryl group optionally substituted with an amino group.

“Co-crystal,” or as sometimes referred to herein “co-precipitate” refers to a solid, preferably a crystalline solid, comprising GGA or a GGA derivative, and urea or thiourea, more preferably, where, the GGA or the GGA derivative reside within the urea or thiourea lattice, such as in channels formed by urea or thiourea.

The term “chiral moiety” refers to a moiety that is chiral. Such a moiety can possess one or more asymmetric centers. Preferably, the chiral moiety is enantiomerically enriched, and more preferably a single enantiomer. Non limiting examples of chiral moieties include chiral carboxylic acids, chiral amines, chiral amino acids, such as the naturally occurring amino acids, chiral alcohols including chiral steroids, and the likes.

The term “cycloalkyl” refers to a monovalent, preferably saturated, hydrocarbyl mono-, bi-, or tricyclic ring having 3-12 ring carbon atoms. While cycloalkyl, refers preferably to saturated hydrocarbyl rings, as used herein, it also includes rings containing 1-2 carbon-carbon double bonds. Nonlimiting examples of cycloalkyl include cyclopropyl, cyclobutyl, cyclopentyl, cyclohexyl, cycloheptyl, adamentyl, and the like. The condensed rings may or may not be non-aromatic hydrocarbyl rings provided that the point of attachment is at a cycloalkyl carbon atom. For example, and without limitation, the following is a cycloalkyl group:

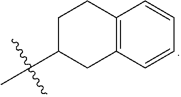 [SMILES: *C1CCc2ccccc2C1]

The term “halo” refers to F, Cl, Br, and/or I.

The term “heteroaryl” refers to a monovalent, aromatic mono-, bi-, or tricyclic ring having 2-14 ring carbon atoms and 1-6 ring heteroatoms selected preferably from N, O, S, and P and oxidized forms of N, S, and P, provided that the ring contains at least 5 ring atoms. Nonlimiting examples of heteroaryl include furan, imidazole, oxadiazole, oxazole, pyridine, quinoline, and the like. The condensed rings may or may not be a heteroatom containing aromatic ring provided that the point of attachment is a heteroaryl atom. For example, and without limitation, the following is a heteroaryl group:

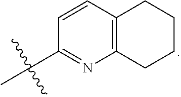 [SMILES: *c1ccc2c(n1)CCCC2]

The term “heterocyclyl” or heterocycle refers to a non-aromatic, mono-, bi-, or tricyclic ring containing 2-10 ring carbon atoms and 1-6 ring heteroatoms selected preferably from N, O, S, and P and oxidized forms of N, S, and P, provided that the ring contains at least 3 ring atoms. While heterocyclyl preferably refers to saturated ring systems, it also includes ring systems containing 1-3 double bonds, provided that they ring is non-aromatic. Nonlimiting examples of heterocyclyl include, azalactones, oxazoline, piperidinyl, piperazinyl, pyrrolidinyl, tetrahydrofuranyl, and tetrahydropyranyl. The condensed rings may or may not contain a non-aromatic heteroatom containing ring provided that the point of attachment is a heterocyclyl group. For example, and without limitation, the following is a heterocyclyl group:

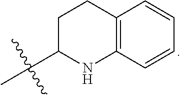 [SMILES: *C1CCc2ccccc2N1]

The term “hydrolyzing” refers to breaking an R^{H}—O—CO—, R^{H}—O—CS—, or an R^{H}—O—SO₂— moiety to an R^{H}—OH, preferably by adding water across the broken bond. A hydrolyzing is performed using various methods well known to the skilled artisan, non limiting examples of which include acidic and basic hydrolysis.

The term “oxo” refers to a C═O group, and to a substitution of 2 geminal hydrogen atoms with a C═O group.

The term “pharmaceutically acceptable” refers to safe and non-toxic for in vivo, preferably, human administration.

The term “pharmaceutically acceptable salt” refers to a salt that is pharmaceutically acceptable.

The term “salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary, and non-limiting cations useful in pharmaceutically acceptable salts include Na, K, Rb, Cs, NH₄, Ca, Ba, imidazolium, and ammonium cations based on naturally occurring amino acids. When the compounds provided and/or utilized herein contain basic functionally, such salts include, without limitation, salts of organic acids, such as caroboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

The term “substantially pure trans isomer” refers to a trans isomer that is by molar amount 95%, preferably 96%, more preferably 99%, and still more preferably 99.5% or more a trans isomer with the rest being the corresponding cis isomer.

### 2. COMPOUNDS

In one aspect, the GGA derivative provided and/or utilized herein is of Formula (I):

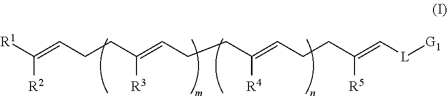

or pharmaceutically acceptable salt thereof, wherein

n is 0, 1 or 2;

m is 0 or 1;

L is a bond or C₁-C₆ alkylene;

G₁ is
—C(═O)H, —CO₂H or an ester or acyl halide thereof;
a 5-14 membered heteroaryl or 5-14 heterocycle containing up to 6 ring heteroatoms, wherein the heteroatom is selected from the group consisting of O, N, S, and oxidized forms of N and S;
wherein the heteroaryl or heterocyclyl is optionally substituted with 1-3 substituents selected from the group consisting of:
hydroxy, oxo, —N(R⁴⁰)₂, C₁-C₆ alkoxy group, C₁-C₆ alkyl group, C₃-C₁₀ cycloalkyl, —CO₂H or an C₁-C₆ alkyl ester or an C₁-C₆ alkyl amide thereof, wherein the cycloalkyl group is optionally substituted with 1-3 C₁-C₆ alkyl groups,
   a 5-9 membered heteroaryl or heterocyclyl containing up to 3 ring heteroatoms, wherein the heteroaryl or heterocyclyl is optionally substituted with 1-3 hydroxy, —N(R⁴⁰)₂, or C₁-C₆ alkyl groups, and
   benzyl or C₆-C₁₀ aryl, optionally substituted with 1-3 substituents selected from the group consisting of C₁-C₆ alkyl, C₁-C₆ alkoxy, hydroxy, and halo groups;
each R¹ and R² are independently C₁-C₆ alkyl, or R¹ and R² together with the carbon atom they are attached to form a C₄-C₇ cycloalkyl ring optionally substituted with 1-3 C₁-C₆ alkyl groups; or a 5-6 membered heterocycle containing up to 3 ring heteroatoms, wherein the heteroatom is selected from the group consisting of O, N, S, and oxidized forms of N and S, and further wherein the heterocyclyl ring is optionally substituted with 1-3 substituents selected from the group consisting of:

hydroxy, oxo, —N(R⁴⁰)₂, C₁-C₆ alkoxy, and C₁-C₆ alkyl,

each of R³, R⁴, and R⁵ independently are hydrogen or C₁-C₆ alkyl;

R⁴⁰ is hydrogen or C₁-C₆ alkyl or 2 R⁴⁰ groups together with the nitrogen atom they are bonded to form a 4-7 membered heterocycle optionally substituted with 1-3 C₁-C₆ alkyl groups.

In some embodiments, if L is a bond, G₁ is not —C(═O)H, —CO₂H or an ester or acyl halide thereof. In some embodiments, if L is a bond or —CH₂—, R¹ and R² are not C₁-C₆ alkyl. In some embodiments, if L is a bond or —CH₂—, R¹ and R² do not combine with the carbon to which they are attached to form a C₄-C₇ cycloalkyl ring. In some embodiments, if L is a bond or —CH₂—, R¹ and R² together form a heterocycle as disclosed above. In some embodiments, the compounds of this invention exclude specific compounds and compounds disclosed generically in U.S. Ser. No. 13/779,568.

In some embodiments, R³, R⁴ and R⁵ are methyl, ethyl, n-propyl, isopropyl, n-butyl, or isobutyl. In preferred embodiments, R³, R⁴ and R⁵ are methyl.

In some embodiments, G₁ is —C(═O)H, —CO₂H or an ester or acyl halide thereof. In some embodiments, G₁ is a ring of formula

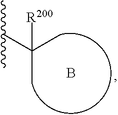

wherein ring B is a 5-10 membered nitrogen-containing heterocycle containing up to 2 additional ring heteroatoms, wherein the heteroatom is selected from the group consisting of O, N, S, and oxidized forms of N and 5; further optionally substituted as disclosed above; and R²⁰⁰ is hydrogen, CO₂H or an C₁-C₆ alkyl ester thereof. In some embodiments, G₁ is a 5-14 membered heteroaryl as disclosed above, optionally substituted as disclosed above. In some embodiments, G₁ is a 5-14 membered heterocycle as disclosed above, optionally substituted as disclosed above.

In some embodiments, L is C₁-alkylene. In some embodiments, L is C₂-alkylene. In some embodiments, L is C₃-C₅ alkylene. In some embodiments, L is —CH₂—. In some embodiments, L is —CH₂CH₂—. In some embodiments, L is —CH₂CH₂CH(CH₃)—. In some embodiments, L is a bond.

In some embodiments, the compound of Formula (I) is of Formula (II):

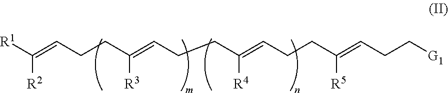

or pharmaceutically acceptable salt thereof.

In some embodiments, n is 0. In some embodiments, n is 1. In some embodiments, n is 2. In some embodiments, m is 0. In some embodiments, m is 1. In some embodiments, n+m is 0. In some embodiments, n+m is 1. In some embodiments, n+m is 2. In some embodiments, n+m is 3.

In some embodiments, R¹, R², R³, R⁴ and R⁵ are methyl, ethyl, n-propyl, isopropyl, n-butyl, or isobutyl. In preferred embodiments, R¹, R², R³, R⁴ and R⁵ are methyl.

In some embodiments, the compound of Formula (I) or (II) is of Formula (IIa):

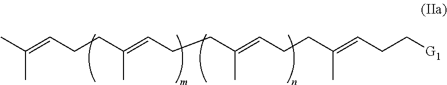

or pharmaceutically acceptable salt thereof.

In some embodiments, for the compound of Formula (II) or (IIa), G is selected from

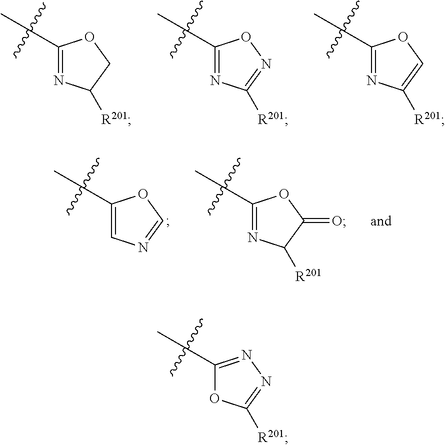

wherein R²⁰¹ is selected from the group consisting of

hydrogen or hydroxy, oxo, —N(R⁴⁰)₂, C₁-C₆ alkoxy group, C₁-C₆ alkyl group, C₃-C₁₀ cycloalkyl, —CO₂H or an C₁-C₆ alkyl ester or an C₁-C₆ alkyl amide thereof, wherein the cycloalkyl group is optionally substituted with 1-3 C₁-C₆ alkyl groups,

a 5-9 membered heteroaryl or heterocyclyl containing up to 3 ring heteroatoms, wherein the heteroaryl or heterocyclyl is optionally substituted with 1-3 hydroxy, —N(R⁴⁰)₂, and C₁-C₆ alkyl group, and

benzyl or C₆-C₁₀aryl, optionally substituted with 1-3 substituents selected from the group consisting of C₁-C₆ alkyl, C₁-C₆ alkoxy, hydroxy, and halo groups;

wherein R⁴⁰ is defined as above.

In some embodiments, R²⁰¹ is hydrogen. In some embodiments, R²⁰¹ is C₁-C₆ alkyl, optionally substituted. In some embodiments, R²⁰¹ is C₁₀ cycloalkyl, optionally substituted. In some embodiments, R²⁰¹ is —CO₂H or an C₁-C₆ alkyl ester or an C₁-C₆ alkyl amide thereof. In some embodiments, R²⁰¹ is heteroaryl, optionally substituted. In some embodiments, R²⁰¹ is heterocyclyl, optionally substituted. In some embodiments, R²⁰¹ is benzyl. In some embodiments, R²⁰¹ is C₆-C₁₀aryl, optionally substituted.

In some embodiments, the compound of Formula (I) is of Formula (III):

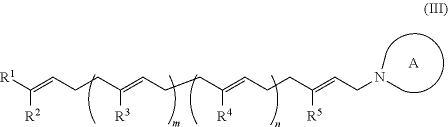

or pharmaceutically acceptable salt thereof, wherein

ring A is a 5-10 membered heteroaryl or heterocycle containing up to 6 ring heteroatoms, wherein the heteroatom is selected from the group consisting of O, N, S, and oxidized forms of N and S, and further wherein the heteroaryl, or heterocyclyl ring is optionally substituted with 1-3 substituents selected from the group consisting of:

hydroxy, oxo, —N(R⁴⁰)₂, C₁-C₆ alkoxy group, C₁-C₆ alkyl group, C₃-C₁₀ cycloalkyl, —CO₂H or an C₁-C₆ alkyl ester or an C₁-C₆ alkyl amide thereof, wherein the alkyl or cycloalkyl group is optionally substituted with 1-3 C₁-C₆ alkyl groups,

a 5-9 membered heteroaryl or heterocyclyl containing up to 3 ring heteroatoms,

wherein the heteroaryl or heterocyclyl is optionally substituted with 1-3 hydroxy, —N(R⁴⁰)₂, and C₁-C₆ alkyl group,

benzyl or C₆-C₁₀aryl, optionally substituted with 1-3 substituents selected from the group consisting of C₁-C₆ alkyl, C₁-C₆ alkoxy, hydroxy, and halo groups.

In some embodiments, the compound of Formula (I) or (III) is of Formula (IIIa):

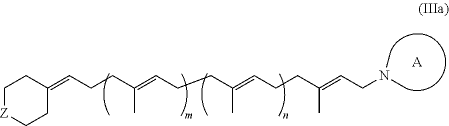

wherein Z is as defined above, and the remaining variables are as defined herein.

In some embodiments, in compounds (III) and (IIIa),

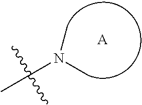

is selected from the group consisting of:

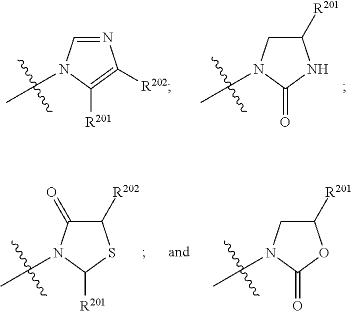

wherein each R²⁰² is defined as R²⁰¹.

In some embodiments, R²⁰² is hydrogen. In some embodiments, R²⁰² is C₁-C₆ alkyl, optionally substituted. In some embodiments, R²⁰² is C₁₀ cycloalkyl, optionally substituted. In some embodiments, R²⁰² is —CO₂H or an C₁-C₆ alkyl ester or an C₁-C₆ alkyl amide thereof. In some embodiments, R²⁰² is heteroaryl, optionally substituted. In some embodiments, R²⁰² is heterocyclyl, optionally substituted. In some embodiments, R²⁰² is benzyl. In some embodiments, R²⁰² is C₆-C₁₀ aryl, optionally substituted.

In some embodiments, R²⁰¹ and R²⁰² are the same. In some embodiments, R²⁰¹ and R²⁰² are different.

In some embodiments, the compound of Formula (I) is of Formula (VI):

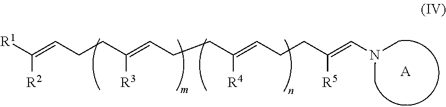

wherein

ring A is a 5-10 membered heteroaryl or heterocycle containing up to 6 ring heteroatoms, wherein the heteroatom is selected from the group consisting of O, N, S, and oxidized forms of N and S, and further wherein the heteroaryl, or heterocyclyl ring is optionally substituted with 1-3 substituents selected from the group consisting of:

hydroxy, oxo, —N(R⁴⁰)₂, C₁-C₆ alkoxy group, C₁-C₆ alkyl group, C₃-C₁₀ cycloalkyl, —CO₂H or an C₁-C₆ alkyl ester or an C₁-C₆ alkyl amide thereof, wherein the alkyl or cycloalkyl group is optionally substituted with 1-3 C₁-C₆ alkyl groups,

a 5-9 membered heteroaryl or heterocyclyl containing up to 3 ring heteroatoms, wherein the heteroaryl or heterocyclyl is optionally substituted with 1-3 hydroxy, —N(R⁴⁰)₂, and C₁-C₆ alkyl group,

benzyl or C₆-C₁₀ aryl, optionally substituted with 1-3 substituents selected from the group consisting of C₁-C₆ alkyl, C₁-C₆ alkoxy, hydroxy, and halo groups.

In some embodiments, the compound of Formula (I) is of Formula (IVa):

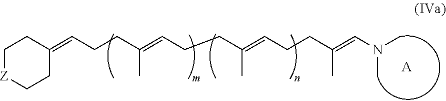

wherein Z is as defined above, and the remaining variables are as defined herein.

In some embodiments, the compound of Formula (I) is of Formula (V):

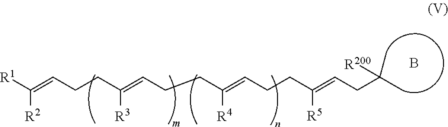

where the variables are as described herein.

In some embodiments, the compound of Formula (I) or (V) is of Formula (Va):

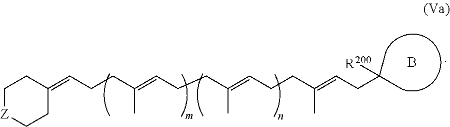

In some embodiments, the compound of Formula (I) is of Formula (VI):

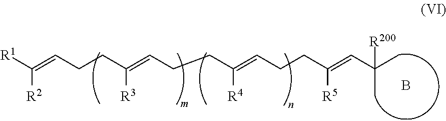

wherein the variables are as defined herein.

In some embodiments,

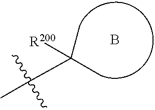

is selected from the group consisting of:

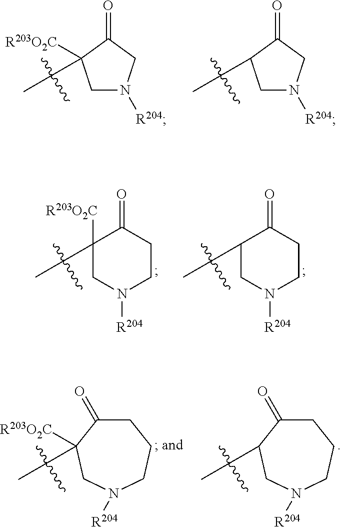

wherein

R²⁰³ is hydrogen or C₁-C₆ alkyl; and

R²⁰⁴ is hydrogen, —CO₂H or a C₁-C₆ alkyl ester or a C₁-C₆ alkyl amide thereof, —SO₂N(R⁴⁰)₂, C₁-C₆ alkyl, C₆-C₁₀ aryl, optionally substituted with 1-3 substituents selected from the group consisting of C₁-C₆ alkyl, C₁-C₆ alkoxy, hydroxy, and halo.

In some embodiments, R²⁰³ is hydrogen. In some embodiments, R²⁰³ is C₁-C₆ alkyl. In some embodiments, R²⁰³ is methyl, ethyl, n-propyl, isopropyl, n-butyl or isobutyl. In preferred embodiments, R²⁰³ is methyl or ethyl.

In some embodiments, R²⁰⁴ is hydrogen. In some embodiments, R²⁰⁴ is —CO₂H. In some embodiments, R²⁰⁴ is a C₁-C₆ alkyl ester. In some embodiments, R²⁰⁴ is a C₁-C₆ alkyl amide. In some embodiments, R²⁰⁴ is —SO₂N(R⁴⁰)₂. In some embodiments, R⁴⁰ are both hydrogen. In some embodiments, R⁴⁰ are both C₁-C₆ alkyl. In some embodiments, R⁴⁰ are both methyl. In some embodiments, R²⁰⁴ is C₁-C₆ alkyl, optionally substituted. In some embodiments, R²⁰⁴ is methyl, ethyl, n-propyl, isopropyl, n-butyl or isobutyl. In some embodiments, R²⁰⁴ is methyl. In some embodiments, R²⁰⁴ is C₆-C₁₀ aryl, optionally substituted. In some embodiments, R²⁰⁴ is phenyl.

In some embodiments, the compound of Formula (I) is of Formula (VII):

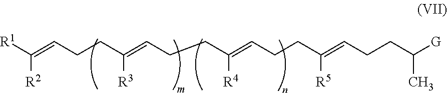

or pharmaceutically acceptable salt thereof.

In some embodiments, the compound of Formula (I) or (VII) is of Formula (VIIa):

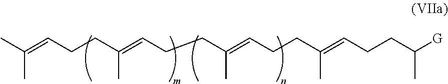

or pharmaceutically acceptable salt thereof.

For compounds of formulas (VII) and (Vila), in some embodiments, G is selected from:

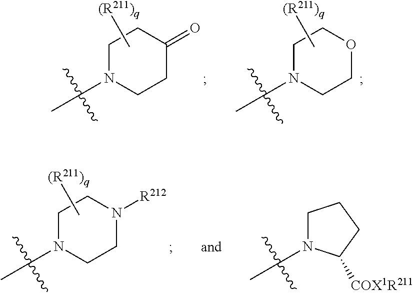

wherein

q is 0, 1, 2, 3 or 4;

X¹ is O or NR²¹⁴

R²¹¹ is selected from the group consisting of
hydrogen,
C₁-C₆ alkyl optionally substituted with 1-3 C₁-C₆ alkyl;
a 5-9 membered heteroaryl containing up to 3 ring heteroatoms, wherein the heteroaryl or heterocyclyl is optionally substituted with 1-3 hydroxy, —N(R⁴⁰)₂, and C₁-C₆ alkyl group, and
C₆-C₁₀ aryl, optionally substituted with 1-3 substituents selected from the group consisting of C₁-C₆ alkyl, C₁-C₆ alkoxy, hydroxy, and halo groups;

R²¹² is selected from the group consisting of
hydrogen, R²¹³, —CO—R²¹³ and —SO₂—R²¹³; wherein

R²¹³ is selected from the group consisting of:
C₁-C₆ alkyl optionally substituted with 1-3 C₁-C₆ alkyl;
a 5-9 membered heteroaryl containing up to 3 ring heteroatoms, wherein the heteroaryl is optionally substituted with 1-3 hydroxy, —N(R⁴⁰)₂, and C₁-C₆ alkyl group, and
C₆-C₁₀ aryl, optionally substituted with 1-3 substituents selected from the group consisting of C₁-C₆ alkyl, C₁-C₆ alkoxy, hydroxy, and halo groups; and

R²¹⁴ is hydrogen or C₁-C₆ alkyl optionally substituted with 1-3 C₁-C₆ alkyl.

In some embodiments, q is 0. In some embodiments, q is 1. In some embodiments, q is 2. In some embodiments, q is 3. In some embodiments, q is 4.

In some embodiments, X¹ is O. In some embodiments, X¹ is NR²¹⁴.

In some embodiments, R²¹¹ is hydrogen. In some embodiments, R²¹¹ is C₁-C₆ alkyl, optionally substituted. In some embodiments, R²¹¹ is a 5-9 membered heteroaryl, optionally substituted. In some embodiments, R²¹¹ is a C₆-C₁₀ aryl, optionally substituted.

In some embodiments, R²¹² is hydrogen. In some embodiments, R²¹² is R²¹³. In some embodiments, R²¹² is —CO—R²¹³. In some embodiments, R²¹² is —SO₂—R²¹³.

In some embodiments, R²¹³ is C₁-C₆ alkyl, optionally substituted. In some embodiments, R²¹³ is a 5-9 membered heteroaryl, optionally substituted. In some embodiments, R²¹³ is a C₆-C₁₀ aryl, optionally substituted.

In some embodiments, R²¹⁴ is hydrogen. In some embodiments, R²¹⁴ is C₁-C₆ alkyl. In some embodiments, R²¹⁴ is methyl.

For the aspects and embodiments disclosed herein, in some embodiments, R¹ and R² are independently C₁-C₆ alkyl. In some embodiments, each R¹, R², R³, R⁴ and R⁵ are independently methyl, ethyl, n-propyl, isopropyl, n-butyl, or isobutyl. In some embodiments, R¹-R⁵ are the same. In preferred embodiments, R¹, R², R³, R⁴ and R⁵ are methyl. In some embodiments, each R³, R⁴ and R⁵ are independently methyl, ethyl, n-propyl, isopropyl, n-butyl, or isobutyl. In preferred embodiments, each R³, R⁴ and R⁵ are methyl.

In some embodiments, R¹ and R² together with the carbon atom they are attached to form a C₄-C₇ cycloalkyl ring optionally substituted with 1-3 C₁-C₆ alkyl groups.

In some embodiments, R¹ and R² together with the carbon atom they are attached to a 5-6 membered heterocycle as disclosed above, and optionally substituted disclosed above. In some preferred embodiments, R¹ and R², with the carbon atom they are attached to, form

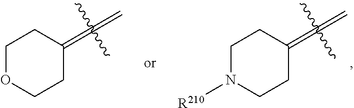

where R²¹⁰ is hydrogen or C₁-C₆ alkyl, CO₂H or an C₁-C₆ alkyl ester thereof. In some embodiments, R²¹⁰ is hydrogen. In some embodiments, R²¹⁰ is C₁-C₆ alkyl. In some embodiments, R²¹⁰ is methyl.

In some preferred embodiments, L is —CH₂— and R¹, R², R³, R⁴ and R⁵ are methyl.

In other preferred embodiments, L is —CH₂CH₂— and R¹, R², R³, R⁴ and R⁵ are methyl.

In still other preferred embodiments, L is —CH₂CH₂CH(CH₃)— and R¹, R², R³, R⁴ and R⁵ are methyl.

In some embodiments, n is 0. In some embodiments, n is 1. In some embodiments, n is 2. In some embodiments, m is 0. In some embodiments, m is 1. In some embodiments, n+m is 0. In some embodiments, n+m is 1. In some embodiments, n+m is 2. In some embodiments, n+m is 3.

In another aspect, the GGA derivative provided and/or utilized herein is of Formula (VIII), (IX), (X), (XI) or (XII):

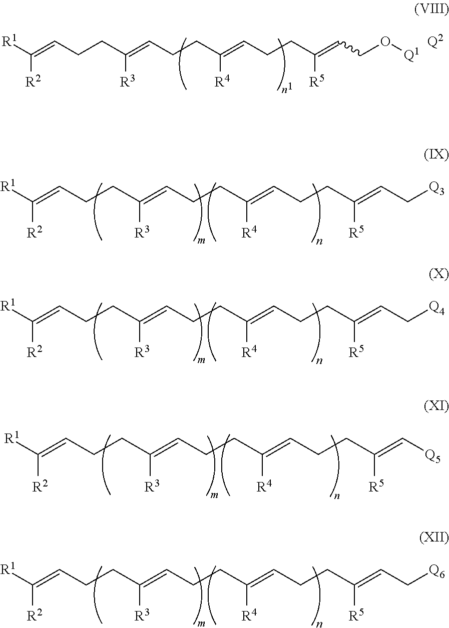

or pharmaceutically acceptable salt thereof, wherein

n¹ is 1 or 2;

n is 0, 1 or 2;

m is 0 or 1;

each R¹ and R² form, together with the carbon atom to which they are attached, a 5-6 membered heterocycle containing up to 3 ring heteroatoms, wherein the heteroatom is selected from the group consisting of O, N, S, and oxidized forms of N and S, and further wherein the heterocyclyl ring is optionally substituted with 1-3 substituents selected from the group consisting of:

hydroxy, oxo, —N(R⁴⁰)₂, C₁-C₆ alkoxy group, and C₁-C₆ alkyl group, wherein the alkyl group is optionally substituted with 1-3 substituents selected from hydroxy, NH₂, —CO₂H or an ester or an amide thereof, wherein R⁴⁰ is defined as above,

each of R³, R⁴, and R⁵ independently are hydrogen or C₁-C₆ alkyl;

Q¹ is —(C═O)—, —(C═S)—, or —S(O₂)—;

Q² is hydrogen, R⁶, —O—R⁶, —NR⁷R⁸, or is a chiral moiety;

Q³ is —OH, —NR²²R²³—X—CO—NR²⁴R²⁵, —X—CS—NR²⁴R²⁵, or —X—SO₂—NR²⁴R²⁵;

Q⁴ is selected from the group consisting of:

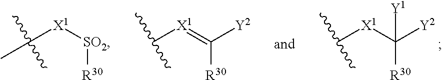

Q⁵ is —C(═O)H, —CO₂H or an ester or acyl halide thereof, wherein the ester is optionally substituted with —CO-phenyl; a 6-10 membered aryl or a 5-14 membered heteroaryl or heterocycle containing up to 6 ring heteroatoms, wherein the heteroatom is selected from the group consisting of O, N, S, and oxidized forms of N and S, and further wherein the aryl, heteroaryl, or heterocyclyl ring is optionally substituted with 1-3 substituents selected from the group consisting of:

hydroxy, oxo, —N(R⁴⁰)₂, C₁-C₆ alkoxy group, and C₁-C₆ alkyl group, wherein the alkyl group is optionally substituted with 1-3 substituents selected from hydroxy, NH₂, —CO₂H or an ester or an amide thereof,

a 5-9 membered heteroaryl containing up to 3 ring heteroatoms, wherein the heteroaryl is optionally substituted with 1-3 hydroxy, —N(R⁴⁰)₂, and C₁-C₆ alkyl group,

benzyl, and phenyl optionally substituted with 1-3 substituents selected from the group consisting of C₁-C₆ alkyl, C₁-C₆ alkoxy, hydroxy, and halo groups; and

when Q₅ is present:

R⁵ and Q₅ together with the intervening carbon atoms form a 6 membered aryl ring, or a 5-8 membered cycloalkenyl ring, or a 5-14 membered heteroaryl or heterocycle, wherein each aryl, cycloalkenyl, heteroaryl, or heterocycle, ring is optionally substituted with 1-2 substituents selected from the group consisting of halo, hydroxy, oxo, —N(R⁴⁰)₂, and C₁-C₆ alkyl;

Q⁶ is selected from the group consisting of:

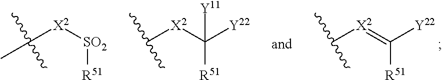

X is —O—, —S—, —NR²⁶—, or —CR²⁷R²⁸;

when X¹ is bonded via a single bond, X¹ is —O—, —NR³¹—, or —CR³²R³³—, and when X¹ is bonded via a double bond, X¹ is —CR³²—;

when X² is bonded via a single bond, X² is —O—, —NR⁵²—, or —CR⁵³R⁵⁴—, and when X² is bonded via a double bond, X² is —CR⁵³—;

Y¹ is hydrogen, —OH or —O—R¹⁰,

Y² is —OH, —OR¹¹ or —NHR¹², or Y¹ and Y² are joined to form an oxo group (═O), an imine group (═NR¹³), a oxime group (═N—OR¹⁴), or a substituted or unsubstituted vinylidene (═CR¹⁶R¹²);

Y¹¹ is hydrogen, —OH or —OR⁵⁵;

Y²² is —OH, —OR⁵⁶, —NHR⁵⁷, or —O—CO—NR⁵⁸R⁵⁹, or Y¹¹ and Y²² are joined to form an oxo group (═O), an imine group (═NR⁶⁰), a oxime group (═N—OR⁶¹), or a substituted or unsubstituted vinylidene (═CR⁶³R⁶⁴);

each R¹ and R² form, together with the carbon atom to which they are attached, a 5-6 membered heterocycle containing up to 3 ring heteroatoms, wherein the heteroatom is selected from the group consisting of O, N, S, and oxidized forms of N and S, and further wherein the heterocyclyl ring is optionally substituted with 1-3 substituents selected from the group consisting of:

hydroxy, oxo, —N(R⁴⁰)₂, C₁-C₆ alkoxy group, and C₁-C₆ alkyl group, wherein the alkyl group is optionally substituted with 1-3 substituents selected from hydroxy, NH₂, —CO₂H or an ester or an amide thereof, R⁴⁰ is discussed as above,

each of R³, R⁴, and R⁵ independently are hydrogen or C₁-C₆ alkyl;

R⁶ is:

C₁-C₆ alkyl, optionally substituted with —CO₂H or an ester thereof, C₁-C₆ alkoxy, oxo, —OH, —CR═CR₂, —C≡CR, C₃-C₁₀ cycloalkyl, C₃-C₈ heterocyclyl, C₆-C₁₀ aryl, C₂-C₁₀heteroaryl, wherein each R independently is hydrogen or C₁-C₆ alkyl;

CO—C₁-C₆ alkyl;

C₃-C₁₀ cycloalkyl;

C₃-C₈ heterocyclyl;

C₆-C₁₀ aryl; or

C₂-C₁₀ heteroaryl;

wherein each cycloalkyl, heterocyclyl, aryl, or heteroaryl is optionally substituted with 1-3 alkyl groups; —CF₃, 1-3 halo, preferably, chloro or fluoro, groups; 1-3 nitro groups; 1-3 C₁-C₆ alkoxy groups; —CO-phenyl; or —NR¹⁸R¹⁹;

each R⁷ and R⁸ are independently hydrogen or defined as R⁶;

R¹⁰ is C₁-C₆ alkyl;

R¹¹ and R¹² are independently C₁-C₆ alkyl, C₃-C₁₀ cycloalkyl, —CO₂R¹⁵, or —CON(R¹⁵)₂, or R¹⁰ and R¹¹ together with the intervening carbon atom and oxygen atoms form a heterocycle optionally substituted with 1-3 C₁-C₆ alkyl groups;

R¹³ is C₁-C₆alkyl or C₃-C₁₀ cycloalkyl optionally substituted with 1-3 C₁-C₆ alkyl groups;

R¹⁴ is hydrogen, C₃-C₈ heterocyclyl, or C₁-C₆alkyl optionally substituted with a —CO₂H or an ester thereof or a C₆-C₁₀ aryl, C₂-C₆ alkenyl, C₂-C₆ alkynyl, C₃-C₁₀cycloalkyl, or a C₃-C₈ heterocyclyl, wherein each cycloalkyl, heterocyclyl, or aryl, is optionally substituted with 1-3 alkyl groups;

each R¹⁵ independently are hydrogen, C₃-C₁₀ cycloalkyl, C₁-C₆ alkyl optionally substituted with 1-3 substituents selected from the group consisting of —CO₂H or an ester thereof, aryl, or C₃-C₈ heterocyclyl, or two R¹⁵ groups together with the nitrogen atom they are bonded to form a 5-7 membered heterocycle;

R¹⁶ is hydrogen or C₁-C₆ alkyl;

R¹⁷ is hydrogen, C₁-C₆ alkyl substituted with 1-3 hydroxy groups, —CHO, or is CO₂H or an ester thereof;

each R¹⁸ and R¹⁹ independently is hydrogen; C₁-C₆ alkyl, optionally substituted with —CO₂H or an ester thereof, C₁-C₆ alkoxy, oxo, —CR═CR₂, —CCR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, or where R¹⁸ and R¹⁹ together with the nitrogen atom they are attached to form a 5-7 membered heterocycle;

each R²² and R²³ independently is hydrogen; C₁-C₆ alkyl, optionally substituted with C₁-C₆ alkoxy; and C₃-C₁₀ cycloalkyl;

each R²⁴ and R²⁵ independently is hydrogen; C₁-C₆ alkyl, optionally substituted with —CO₂H or an ester thereof, C₁-C₆ alkoxy, oxo, —CR═CR₂, —CCR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, or R²⁴ and R²⁵ together with the nitrogen atom they are attached to form a 5-7 membered heterocycle;

R²⁶ is hydrogen or together with R²⁴ or R²⁵ and the intervening atoms form a 5-7 membered heterocyclic ring optionally substituted with 1-3 C₁-C₆ alkyl groups;

each R²⁷ and R²⁸ independently are hydrogen, C₁-C₆ alkyl, —COR⁸¹ or —CO₂R⁸¹, or R²⁷ together with R²⁴ or R²⁵ and the intervening atoms form a 5-7 membered heterocyclyl ring optionally substituted with 1-3 C₁-C₆ alkyl groups;

R³⁰ is C₁-C₆ alkyl optionally substituted with 1-3 alkoxy or 1-5 halo group, C₂-C₆ alkenyl, C₂-C₆ alkynyl, C₃-C₁₀ cycloalkyl, C₆-C₁₀ aryl, C₃-C₈ heterocyclyl, or C₂-C₁₀ heteroaryl, wherein each cycloalkyl or heterocyclyl is optionally substituted with 1-3 C₁-C₆ alkyl groups, or wherein each aryl or heteroaryl is independently substituted with 1-3 C₁-C₆ alkyl or nitro groups, or R³⁰ is —NR³⁴R³⁵;

R³¹ is hydrogen or together with R³⁰ and the intervening atoms form a 5-7 membered ring optionally substituted with 1-3 C₁-C₆ alkyl groups;

each R³² and R³³ independently are hydrogen, C₁-C₆ alkyl, —COR⁸¹ or —CO₂R⁸¹, or R³² together with R³⁰ and the intervening atoms form a 5-7 membered cycloalkyl or heterocyclyl ring optionally substituted with oxo or 1-3 C₁-C₆ alkyl groups;

each R³⁴ and R³⁵ independently is hydrogen, C₁-C₆ alkyl, optionally substituted with —CO₂H or an ester thereof, C₃-C₁₀ cycloalkyl, C₃-C₈ heterocyclyl, C₆-C₁₀ aryl, or C₂-C₁₀ heteroaryl, or is C₃-C₁₀ cycloalkyl, C₃-C₈ heterocyclyl, C₆-C₁₀ aryl, or C₂-C₁₀ heteroaryl, wherein each cycloalkyl, heterocyclyl, aryl, or heteroaryl is optionally substituted with 1-3 alkyl groups, or R³⁴ and R³⁵ together with the nitrogen atom they are attached to form a 5-7 membered heterocycle;

R⁵¹ is C₁-C₆ alkyl, C₁-C₆ alkyl substituted with 1-3 alkoxy or 1-5 halo groups, C₂-C₆ alkenyl, C₂-C₆ alkynyl, C₃-C₁₀ cycloalkyl, C₃-C₈ heterocyclyl, C₆-C₁₀ aryl, C₂-C₁₀ heteroaryl, or —NR⁶⁵R⁶⁶, wherein each cycloalkyl or heterocyclyl is optionally substituted with 1-3 C₁-C₆ alkyl groups, and wherein each aryl or heteroaryl is optionally substituted independently with 1-3 nitro and C₁-C₆ alkyl groups;

R⁵² is hydrogen or together with R⁵¹ and the intervening atoms form a 5-7 membered ring optionally substituted with 1-3 C₁-C₆ alkyl groups;

each R⁵³ and R⁵⁴ independently are hydrogen, C₁-C₆ alkyl, —COR⁸¹, —CO₂R⁸¹, or —CONHR⁸², or R⁵³ together with R⁵¹ and the intervening atoms form a 5-7 membered cycloalkyl or heterocyclyl ring optionally substituted with 1-3 C₁-C₆ alkyl groups;

R⁵⁵ is C₁-C₆ alkyl;

each R⁵⁶ and R⁵² independently are C₁-C₆ alkyl, C₃-C₁₀ cycloalkyl, —CO₂R⁶², or —CON(R⁶²)₂; or R⁵⁵ and R⁵⁶ together with the intervening carbon atom and oxygen atoms form a heterocycle optionally substituted with 1-3 C₁-C₆ alkyl groups;

R⁵⁸ is: C₃-C₁₀ cycloalkyl, C₁-C₆ alkyl optionally substituted with —OH, CO₂H or an ester thereof, or C₃-C₁₀ cycloalkyl,

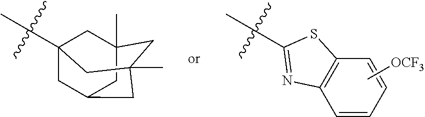

R⁵⁹ is hydrogen or C₁-C₆ alkyl;

R⁶⁰ is C₁-C₆ alkyl or C₃-C₁₀ cycloalkyl optionally substituted with 1-3 C₁-C₆ alkyl groups, or is:

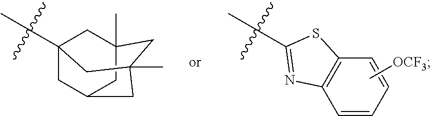

R⁶¹ is hydrogen, C₃-C₈ heterocyclyl, or C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;

each R⁶² independently are hydrogen, C₃-C₁₀ cycloalkyl, C₁-C₆ alkyl optionally substituted with 1-3 substituents selected from the group consisting of —CO₂H or an ester thereof, aryl, C₃-C₈ heterocyclyl, or two R⁶² groups together with the nitrogen atom they are bonded to form a 5-7 membered heterocycle;

R⁶³ is hydrogen or C₁-C₆ alkyl;

R⁶⁴ is hydrogen, C₁-C₆ alkyl substituted with 1-3 hydroxy groups, —CHO, or is CO₂H or an ester thereof;

one or both of R⁶⁵ and R⁶⁶ independently are hydrogen, C₁-C₆ alkyl, optionally substituted with —CO₂H or an ester thereof, C₃-C₁₀ cycloalkyl, C₃-C₈ heterocyclyl, C₂-C₁₀ aryl, or C₂-C₁₀ heteroaryl, or is C₃-C₁₀ cycloalkyl, C₃-C₈ heterocyclyl, C₆-C₁₀aryl, or C₂-C₁₀ heteroaryl, wherein each cycloalkyl, heterocyclyl, aryl, or heteroaryl is optionally substituted with 1-3 alkyl groups, or R⁶⁵ and R⁶⁶ together with the nitrogen atom they are bonded to form a 5-7 membered heterocycle, and if only one of R⁶⁵ and R⁶⁶ are defined as above, then the other one is

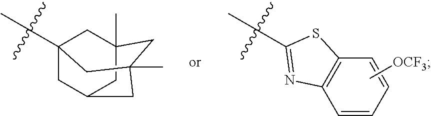

R⁸¹ is C₁-C₆ alkyl; and

R⁸² is:

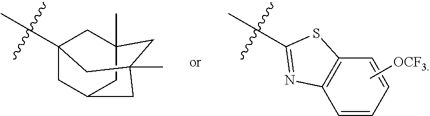

In one embodiment, when X² is bonded via a single bond, and R⁵³ or R⁵⁴ is not —CONHR⁸², Y¹¹ and Y²² are joined to form an imine group (═NR⁶⁰), and R⁶⁰ is:

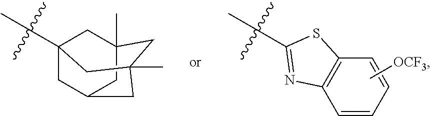

or Y²² is —O—CO—NR⁵⁸R⁵⁹; or

when Q₆ is:

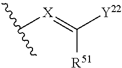

and R⁵³ is not —CONHR⁸², Y²² is —O—CO—NR⁵⁸R⁵⁹;

or provided that, when Q₆ is —O—CO—NR⁶⁵R⁶⁶, then at least one of R⁶⁵ and R⁶⁶ is:

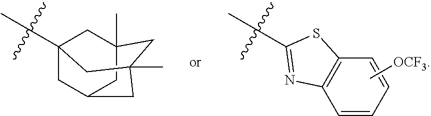

In some embodiments, R¹ and R² are together with the carbon atom they are attached to form

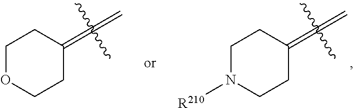

R²¹⁰ is hydrogen or C₁-C₆ alkyl, CO₂H or an C₁-C₆ alkyl ester thereof.

In some embodiments, R³, R⁴ and R⁵ are methyl.

In some embodiments, n is 0. In some embodiments, n is 1. In some embodiments, n is 2. In some embodiments, m is 0. In some embodiments, m is 1. In some embodiments, n+m is 0. In some embodiments, n+m is 1. In some embodiments, n+m is 2. In some embodiments, n+m is 3.

### 3. PHARMACEUTICAL COMPOSITIONS

In another aspect, this invention is also directed to pharmaceutical compositions comprising at least one pharmaceutically acceptable excipient and an effective amount of the trans-isomer compound of a GGA derivative according to this invention.

Pharmaceutical compositions can be formulated for different routes of administration. Although compositions suitable for oral delivery will probably be used most frequently, other routes that may be used include intravenous, intraarterial, pulmonary, rectal, nasal, vaginal, lingual, intramuscular, intraperitoneal, intracutaneous, transdermal, intracranial, and subcutaneous routes. Other dosage forms include tablets, capsules, pills, powders, aerosols, suppositories, parenterals, and oral liquids, including suspensions, solutions and emulsions. Sustained release dosage forms may also be used, for example, in a transdermal patch form. All dosage forms may be prepared using methods that are standard in the art (see e.g., Remington's Pharmaceutical Sciences, 16^{th} ed., A. Oslo editor, Easton Pa. 1980).

The compositions are comprised of in general, a GGA derivative or a trans-isomer compound of a GGA derivative or a mixture thereof in combination with at least one pharmaceutically acceptable excipient. Acceptable excipients are non-toxic, aid administration, and do not adversely affect the therapeutic benefit of the compound of this invention. Such excipients may be any solid, liquid, semi-solid or, in the case of an aerosol composition, gaseous excipient that is generally available to one of skill in the art. Pharmaceutical compositions in accordance with the invention are prepared by conventional means using methods known in the art.

The compositions disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerin and the like.

Solid pharmaceutical excipients include starch, cellulose, hydroxypropyl cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc.

The concentration of the excipient is one that can readily be determined to be effective by those skilled in the art, and can vary depending on the particular excipient used. The total concentration of the excipients in the solution can be from about 0.001% to about 90% or from about 0.001% to about 10%.

In certain embodiments of this invention, there is provided a pharmaceutical composition comprising the compound of formulas (I) and (VIII)-(XXII) and α-tocopherol. A related embodiment provides for a pharmaceutical composition comprising the compound of formulas (I) and (VIII)-(XXII), α-tocopherol, and hydroxypropyl cellulose. In another embodiment, there is provided a pharmaceutical composition comprising the compound of formulas (I) and (VIII)-(XXII), α-tocopherol, and gum arabic. In a further embodiment, there is a pharmaceutical composition comprising the compound of formulas (I) and (VIII)-(XXII), and gum arabic. In a related embodiment, there is provided the compound of formulas (I) and (VIII)-(XXII), gum arabic and hydroxypropyl cellulose.

When α-tocopherol is used alone or in combination with other excipients, the concentration by weight can be from about 0.001% to about 1% or from about 0.001% to about 0.005%, or from about 0.005% to about 0.01%, or from about 0.01% to about 0.015%, or from about 0.015% to about 0.03%, or from about 0.03% to about 0.05%, or from about 0.05% to about 0.07%, or from about 0.07% to about 0.1%, or from about 0.1% to about 0.15%, or from about 0.15% to about 0.3%, or from about 0.3% to about 0.5%, or from about 0.5% to about 1% by weight. In some embodiments, the concentration of α-tocopherol is about 0.001% by weight, or alternatively about 0.005%, or about 0.008%, or about 0.01%, or about 0.02%, or about 0.03%, or about 0.04%, or about 0.05% by weight.

When hydroxypropyl cellulose is used alone or in combination with other excipients, the concentration by weight can be from about 0.1% to about 30% or from about 1% to about 20%, or from about 1% to about 5%, or from about 1% to about 10%, or from about 2% to about 4%, or from about 5% to about 10%, or from about 10% to about 15%, or from about 15% to about 20%, or from about 20% to about 25%, or from about 25% to about 30% by weight. In some embodiments, the concentration of hydroxypropyl cellulose is about 1% by weight, or alternatively about 2%, or about 3%, or about 4%, or about 5%, or about 6%, or about 7%, or about 8%, or about 10%, or about 15% by weight.

When gum arabic is used alone or in combination with other excipients, the concentration by weight can be from about 0.5% to about 50% or from about 1% to about 20%, or from about 1% to about 10%, or from about 3% to about 6%, or from about 5% to about 10%, or from about 4% to about 6% by weight. In some embodiments, the concentration of hydroxypropyl cellulose is about 1% by weight, or alternatively about 2%, or about 3%, or about 4%, or about 5%, or about 6%, or about 7%, or about 8%, or about 10%, or about 15% by weight.

The concentration of GGA derivative, or its trans isomer can be from about 1 to about 99% by weight in the pharmaceutical compositions provided herein. In other embodiments, the concentration of the trans isomer can be from about 1 to about 75%, or alternatively, from about 1 to about 40%, or alternatively, from about 1 to about 30%, or alternatively, from about 1 to about 25%, or alternatively, from about 1 to about 20%, or alternatively, from about 2 to about 20%, or alternatively, from about 1 to about 10%, or alternatively, from about 10 to about 20%, or alternatively, from about 10 to about 15% by weight in the pharmaceutical composition. In certain embodiments, the concentration of geranylgeranyl acetone in the pharmaceutical composition is about 5% by weight, or alternatively, about 10%, or about 20%, or about 1%, or about 2%, or about 3%, or about 4%, or about 6%, or about 7%, or about 8%, or about 9%, or about 11%, or about 12%, or about 14%, or about 16%, or about 18%, or about 22%, or about 25%, or about 26%, or about 28%, or about 30%, or about 32%, or about 34%, or about 36%, or about 38%, or about 40%, or about 42%, or about 44%, or about 46%, or about 48%, or about 50%, or about 52%, or about 54%, or about 56%, or about 58%, or about 60%, or about 64%, or about 68%, or about 72%, or about 76%, or about 80% by weight.

In one embodiment, this invention provides sustained release formulations such as drug depots or patches comprising an effective amount of GGA derivative. In another embodiment, the patch further comprises gum Arabic or hydroxypropyl cellulose separately or in combination, in the presence of alpha-tocopherol. Preferably, the hydroxypropyl cellulose has an average MW of from 10,000 to 100,000. In a more preferred embodiment, the hydroxypropyl cellulose has an average MW of from 5,000 to 50,000. The patch contains, in various embodiments, an amount of GGA derivative, preferably the 5E,9E,13E isomer of it, which is sufficient to maintain a therapeutically effective amount GGA derivative in the plasma for about 12 hours. In one embodiment, the GGA derivative comprises at least 80%, at least 85%, at least 90%, at least 95%, or at least 99% of the 5E,9E,13E isomer of GGA derivative.

Compounds and pharmaceutical compositions of this invention may be used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

In some embodiments, a compound of this invention can be used as an adjunct to conventional drug therapy.

### 5. SYNTHETIC METHODS

This invention provides a synthetic method comprising one or more of the following steps:

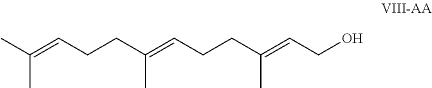 [SMILES: CC(C)=CCC/C(C)=C/CC/C(C)=C/CO]

(i) reacting a compound of formula III-AA under halogenation conditions to provide a compound of formula IX-AA;

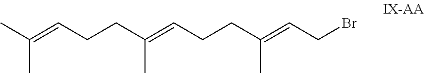 [SMILES: CC(C)=CCC/C(C)=C/CC/C(C)=C/CBr]

(ii) reacting the compound of formula IX-AA with alkyl acetoacetate under alkylation conditions to provide a compound of formula X-AA, where the stereochemistry at stereogenic center can be a racemic, R or S configuration:

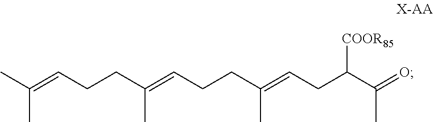

(iii) reacting the compound of formula V-AA under hydrolysis and decarboxylation conditions to provide a compound of formula XI-AA:

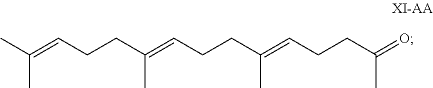 [SMILES: CC(=O)CC/C=C(\C)CC/C=C(\C)CCC=C(C)C]

(iv) reacting the compound of formula XI-AA with a compound of formula XII-AA:

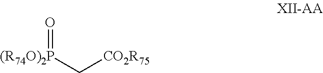

wherein R₇₄, R₇₅, R₈₅ and each R₈₆ independently are alkyl or substituted or unsubstituted aryl, under olefination conditions to selectively provide a compound of formula XIII-AA:

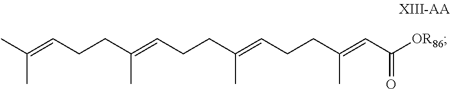

(v) reacting the compound of formula XIII-AA under reduction conditions to provide a compound of formula XIV-AA

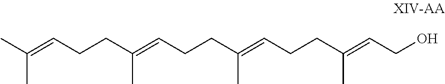 [SMILES: CC(C)=CCC/C(C)=C/CC/C(C)=C/CC/C(C)=C/CO]

Compound VIII-AA is combined with at least an equimolar amount of a halogenating agent typically in an inert solvent. As used in this application, an “inert solvent” is a solvent that does not react under the reaction conditions in which it is employed as a solvent. The reaction is typically run at a temperature of about 0° C. to 20° C. for a period of time sufficient to effect substantial completion of the reaction. Suitable solvents include, by way of example only, diethyl ether, acetonitrile, and the like. Suitable halogenating agents include PBr₃ or PPh₃/CBr₄. After reaction completion, the resulting product, compound IX-AA, can be recovered under conventional conditions such as extraction, precipitation, filtration, chromatography, and the like or, alternatively, used in the next step of the reaction without purification and/or isolation.

Compound IX-AA is combined with at least an equimolar amount of an alkyl acetoacetate, in the presence of a base and an inert solvent. The reaction is typically run initially at 0° C., and then warmed up to room temperature for a period of time sufficient to effect substantial completion of the reaction. Suitable solvents include, by way of example only, various alcohols, such as ethanol, dioxane, and mixtures thereof. Suitable bases include, by way of example only, alkali metal alkoxides, such as sodium ethoxide.

Compound X-AA is reacted with at least an equimolar amount, preferably, an excess of aqueous alkali. The reaction is typically run at about 40 to 80° C. and preferably about 80° C. for a period of time sufficient to effect substantial completion of the reaction. Suitable solvents include, by way of examples only, alcohols, such as methanol, ethanol, and the like.

Compound XI-AA is combined with at least an equimolar amount, preferably, an excess of a compound of formula XII-AA, and at least an equimolar amount, preferably, an excess of base, in an inert solvent. The reaction is typically run, initially at about −30° C. for about 1-2 hours, and at room temperature for a period of time sufficient to effect substantial completion of the reaction. Suitable solvents include, by way of examples only tetrahydrofuran, dioxane, and the like. Suitable bases include, by way of example only, alkali metal hydrides, such as sodium hydride, or potassium hexamethyldisilazide (KHMDS), or potassium tertiary butoxide (^{t}BuOK).

Compound XIII-AA is combined with a reducing agent in an inert solvent. The reaction is typically run at about 0° C. for about 15 minutes, and at room temperature for a period of time sufficient to effect substantial completion of the reaction. Suitable reducing agents include, without limitation, LiAlH₄. Suitable solvents include, by way of examples only diethyl ether, tetrahydrofuran, dioxane, and the like.

As will be apparent to the skilled artisan, after reaction completion, the resulting product, can be recovered under conventional conditions such as precipitation, filtration, chromatography, and the like or, alternatively, used in the next step of the reaction without purification and/or isolation.

In some embodiments, the method further comprises repeating steps (i), (ii), and (iii) sequentially with compound of formula XIII-AA to provide the compound of formula VI-B, wherein m is 2.

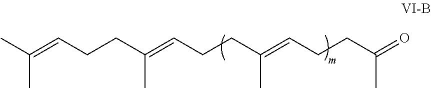

In another embodiment, the method or procedure further comprises repeating steps (i), (ii), (iii), (iv), and (v), sequentially, 1-3 times.

In another of its synthetic method aspects, there is provided a method comprising one or more of the following steps:

(i) reacting a compound of formula VIII-B:

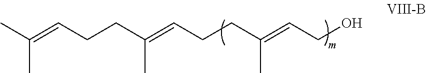

wherein m is 1-3, under halogenation conditions to provide a compound of formula IX-B:

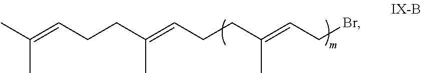

(ii) reacting the compound of formula IX-B with alkyl acetoacetates, under alkylating conditions to provide a compound of formula X-B, where the stereochemistry at sterogenic center can be a racemic, R or S configuration:

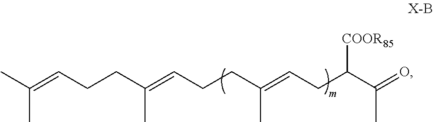

wherein R³¹ alkyl is substituted or unsubstituted alkyl
(iii) reacting a compound of formula X-B under hydrolysis and decarboxylation conditions to provide a compound of formula XI-B:

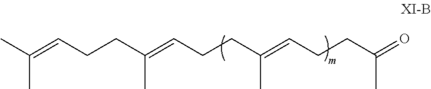

In another of its synthetic method aspects, this invention provides a method comprising step (i) or step (ii) or steps (i)+(ii):

(i) reacting a compound of formula XV-C:

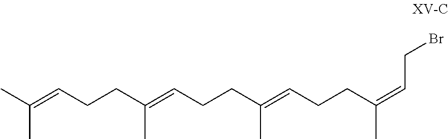 [SMILES: CC(C)=CCC/C(C)=C/CC/C(C)=C/CC/C(C)=C\CBr]

with alkyl acetoacetate under alkylating conditions to provide a compound of formula XVI-C, the stereochemistry at stereogenic center can be a racemic, R or S configuration:

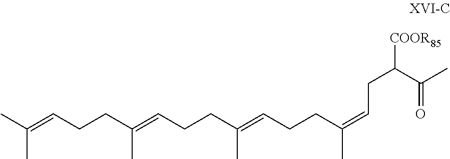

wherein R₃₁ is as defined herein, and
(ii) reacting the compound XVI-C obtained under hydrolysis and decarboxylation conditions to provide a compound of formula VII-AA:

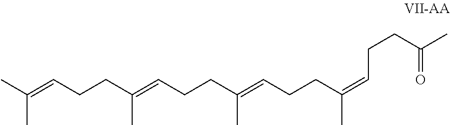 [SMILES: CC(=O)CC/C=C(/C)CC/C=C(\C)CC/C=C(\C)CCC=C(C)C]

As will be apparent to the skilled artisan, the various reaction steps leading to compound XI-B or to the 5Z isomer are performed in the manner described hereinabove.

In another of its synthetic method aspects, this invention provides a method comprising reacting a ketal compound of formula XVII-AA:

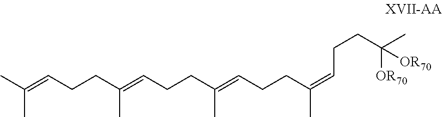

wherein each R₇₀ independently is C₁-C₆ alkyl, or two R₇₀ groups together with the oxygen atoms they are attached to form a 5 or 6 membered ring, which ring is optionally substituted with 1-3, preferably 1-2, C₁-C₆ alkyl groups, under hydrolysis conditions to provide a compound of formula II-AA.

The ketal is combined with at least a catalytic amount, such as, 1-20 mole % of an aqueous acid, preferably, an aqueous mineral acid in an inert solvent. The reaction is typically run about 25° C. to about 80° C., for a period of time sufficient to effect substantial completion of the reaction. Suitable acids include, without limitation, HCl, H₂SO₄, and the like. Suitable solvents include alcohols, such as methanol, ethanol, tetrahydrofuran, and the like.

In another embodiment, this invention provides a method comprising reacting a compound of formula XVI-AA:

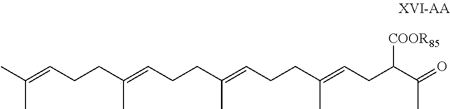

under hydrolysis and subsequently decarboxylation conditions to form a compound of formula I:

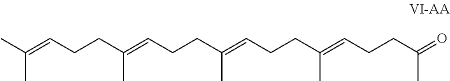 [SMILES: CC(=O)CC/C=C(\C)CC/C=C(\C)CC/C=C(\C)CCC=C(C)C]

Alternatively, reacting compound of formula XII-AA with XV-AA followed by in situ hydrolysis and decarboxylation of compound with formula XVI-AA can afford the compound of formula VI-AA.

In another embodiment, this invention provides a method comprising reacting a compound of formula XVI-C:

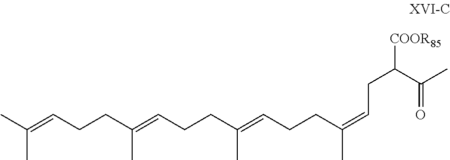

under hydrolysis and subsequent decarboxylation conditions to form the compound of formula VII-AA

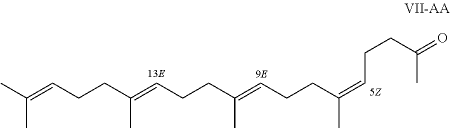 [SMILES: CC(=O)CC/C=C(/C)CC/C=C(\C)CC/C=C(\C)CCC=C(C)C]

Hydrolysis and decarboxylation conditions useful in these methods will be apparent to the skilled artisan upon reading this disclosure.

It will also be apparent to the skilled artisan that the methods further employ routine steps of separation or purification to isolate the compounds, following methods such as chromatography, distillation, or crystallization.

In some embodiments, this invention provides compositions comprising co-crystals or co-precipitates of the GGA derivatives described herein (including salts and tauotomers thereof) with urea and/or thiourea, and processes related to such co-crystals. Preferably, the co-crystals include the all-trans (hereinafter “trans”) form or substantially the trans form of the GGA derivative.

### Synthesis of GGA Derivatives

Certain methods for making GGA or certain GGA derivatives provided and/or utilized herein are described in PCT publication no. WO 2012/031028, PCT application no. PCT/US2012/027147, and U.S. Ser. No. 13/779,568 each of which are incorporated herein by reference in its entirety. Other GGA derivatives can be prepared by appropriate substitution of reagents and starting materials, as will be well known to the skilled artisan upon reading this disclosure.

The reactions are preferably carried out in a suitable inert solvent that will be apparent to the skilled artisan upon reading this disclosure, for a sufficient period of time to ensure substantial completion of the reaction as observed by thin layer chromatography, ¹H-NMR, etc. If needed to speed up the reaction, the reaction mixture can be heated, as is well known to the skilled artisan. The final and the intermediate compounds are purified, if necessary, by various art known methods such as crystallization, precipitation, column chromatography, and the likes, as will be apparent to the skilled artisan upon reading this disclosure.

Some compounds provided and/or utilized in this invention are synthesized according to methods described in the schemes below:

For example, provided below are representative synthetic routes to compounds having a one carbon L group and heteroaryl or heterocyclic G₁ groups:

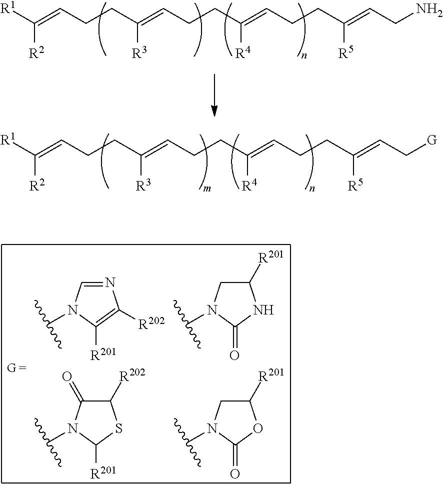

wherein the variables are as defined herein. Methods for cyclizing a primary amine will be apparent to the skilled artisan in view of this disclosure.

Provided below are representative synthetic routes to compounds having a one carbon L group and heteroaryl or heterocyclic G₁ groups:

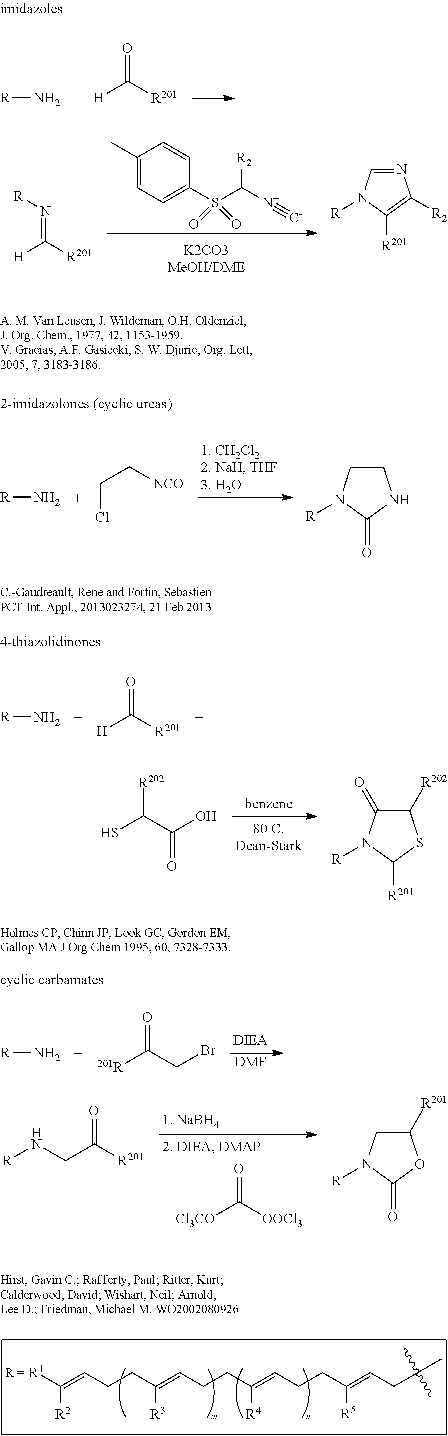

wherein the variables are as defined herein.

Provided below are representative synthetic routes to compounds having a one carbon L group and heterocyclic G₁ groups:

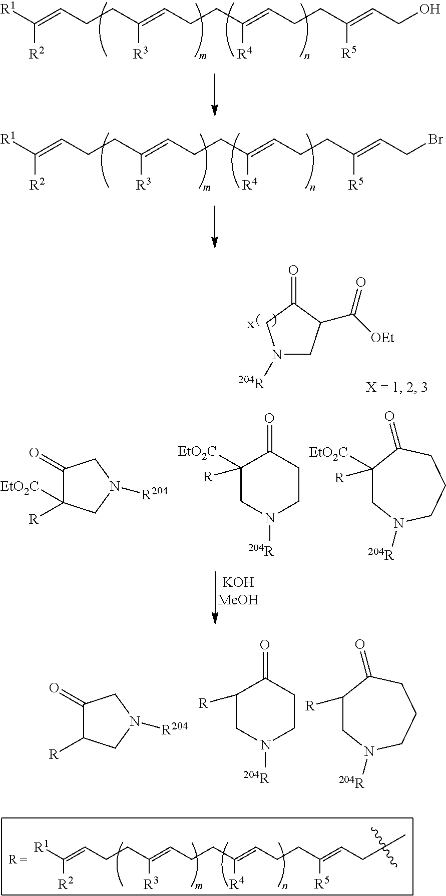

wherein the variables are as defined herein.

Provided below are representative synthetic routes to compounds having a one carbon L group:

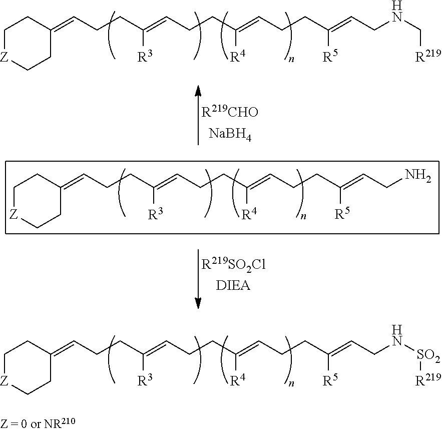

wherein

R²¹⁰ is hydrogen or C₁-C₆ alkyl, CO₂H or an C₁-C₆ alkyl ester thereof.

R²¹⁴ is C₁-C₆ alkyl or C₆-C₁₀ aryl, wherein each alkyl or aryl is optionally substituted with 1-3 alkyl or halo groups; and the remaining variables are as defined herein.

Provided below are representative synthetic routes to compounds having a two carbon L group:

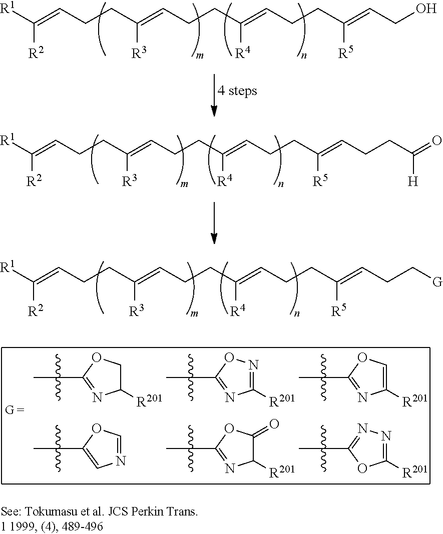

wherein the variables are as defined herein.

Provided below are representative synthetic routes to compounds having a two carbon L group:

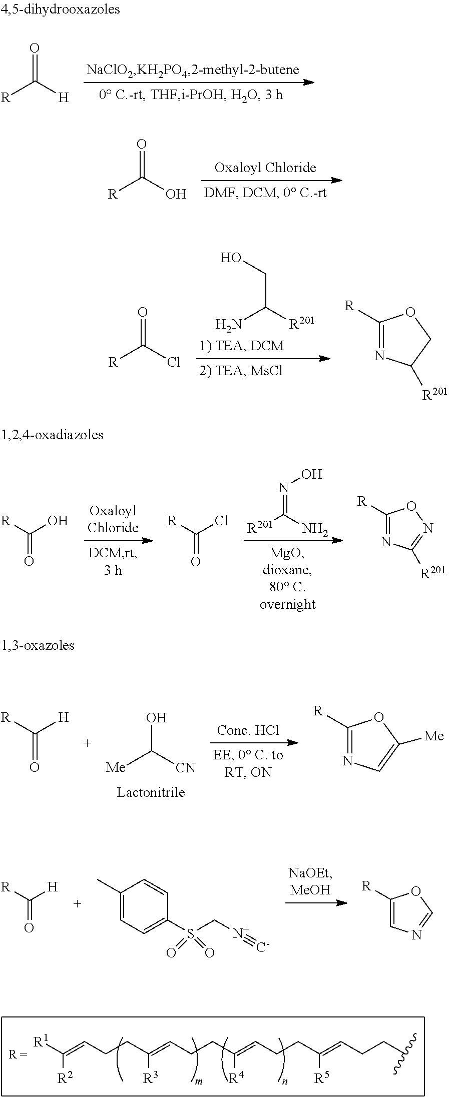

wherein the variables are as defined herein.

Provided below are representative synthetic routes to compounds having a two carbon L group:

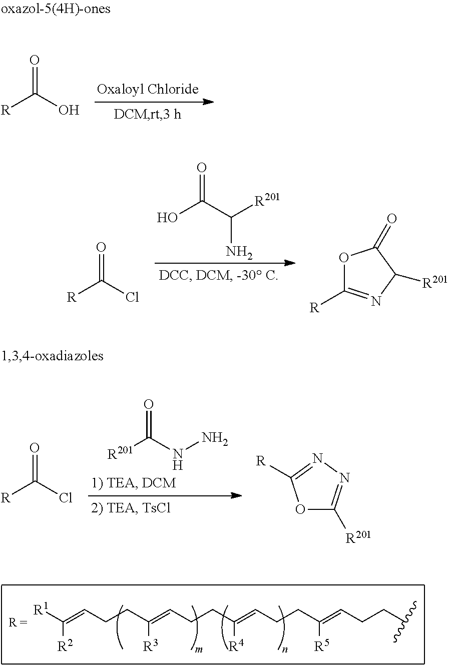

wherein the variables are as defined herein.

Provided below are representative synthetic routes to compounds having a two carbon L group:

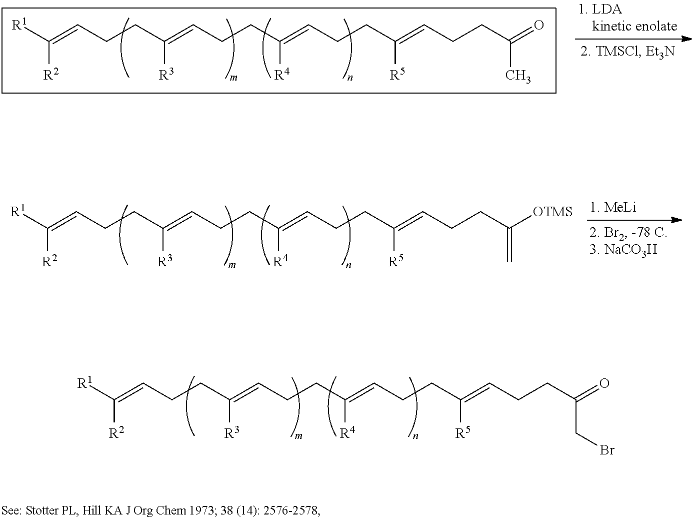

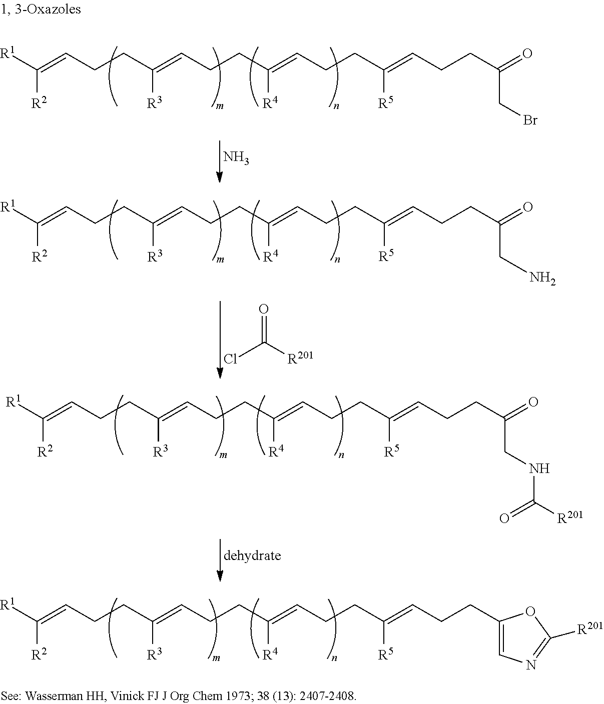

wherein the variables are as defined herein.

Provided below are representative synthetic routes to compounds having a two carbon L group:

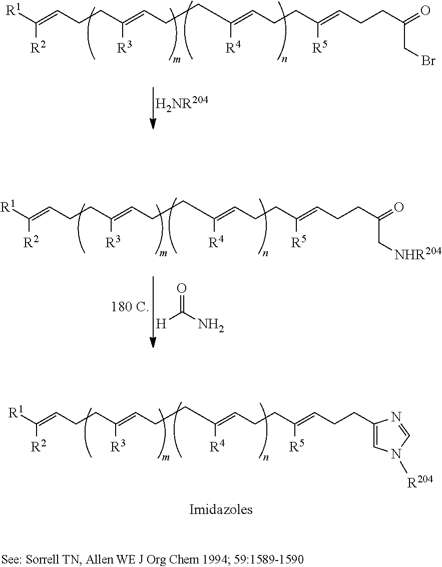

wherein the variables are as defined herein.

Provided below are representative synthetic routes to compounds having a two carbon L group:

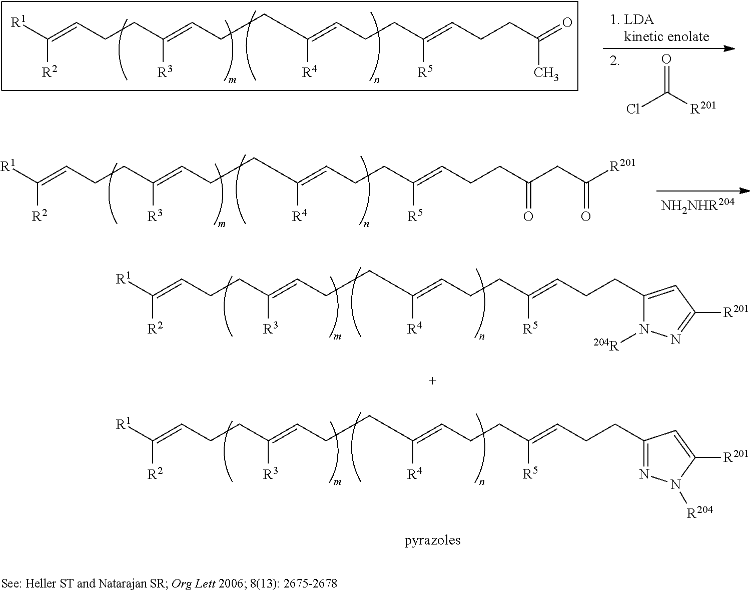

wherein the variables are as defined herein.

Provided below are representative synthetic routes to compounds having a two carbon L group:

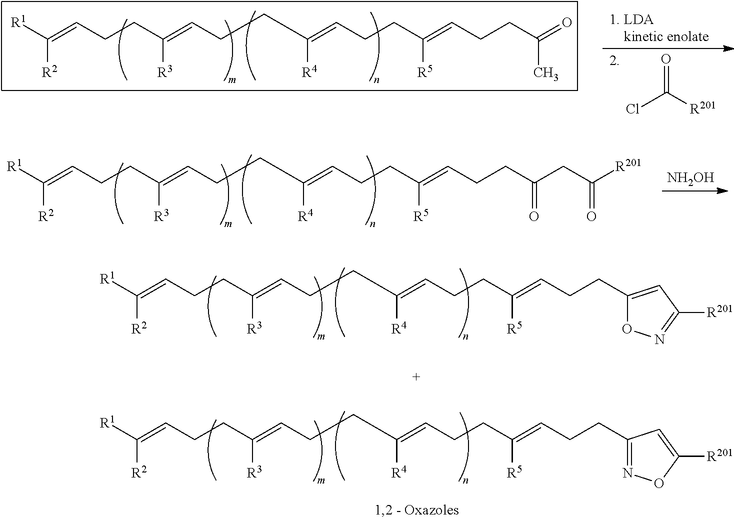

wherein the variables are as defined herein.

Provided below are representative synthetic routes to compounds having a two carbon L group:

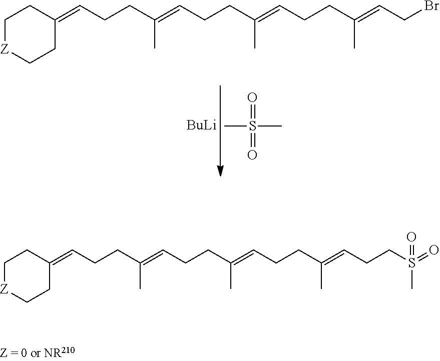

wherein the variables are as defined herein.

Provided below are representative synthetic precursors to compounds having a two or three carbon L group:

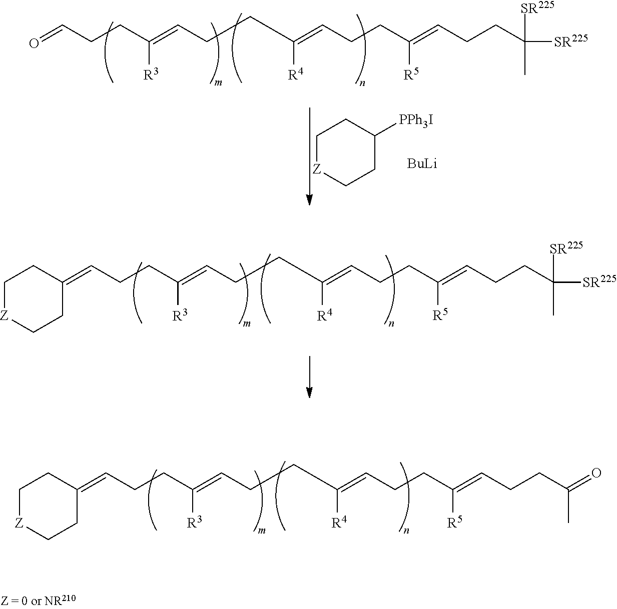

wherein each R²²⁵ is C₁-C₆ alkyl or both R²²⁵ are combine with the sulfur and carbon atoms to form a 5- or 6-membered heterocyclic ring and the remaining variables are as defined herein. The dithiaketal intermediate employed above is synthesized as schematically shown below.

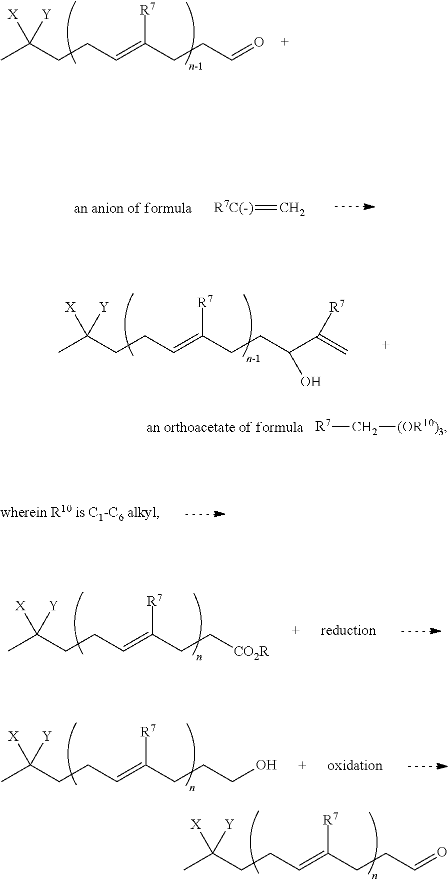

wherein in exemplary embodiments, X and Y are each independently SR²²⁵, each R⁶ is independently C₁-C₆ alkyl, each X¹ and X² are independently O, or S; q is 1 or 2; each X³ is independently C₁-C₆ alkyl; t is 0, 1, 2, or 3, each of R′ independently is H or C₁-C₆ alkyl; and n is 1-5. 7. In some embodiments, R¹-R⁵ are methyl. In some embodiments, R⁷ is methyl.

Provided below are representative synthetic precursors to compounds having a two or three carbon L group:

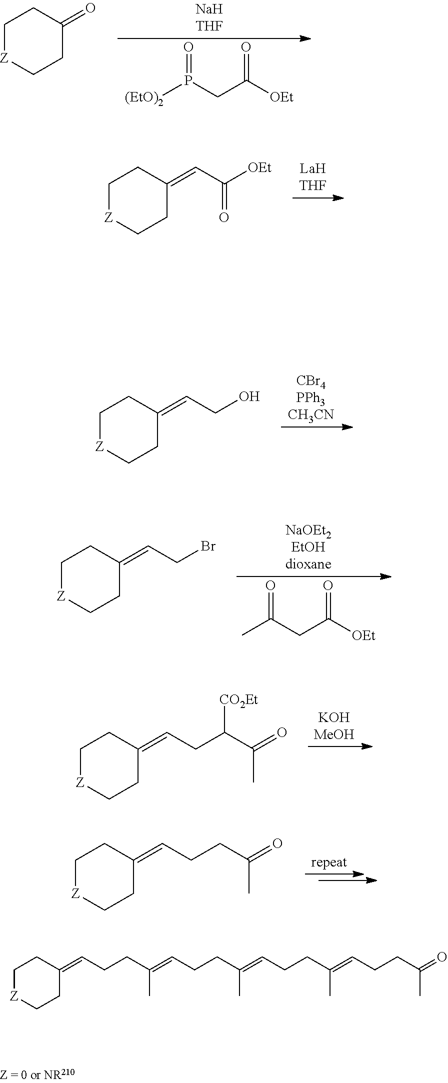

Provided below are representative synthetic routes to compounds having a three carbon L group.

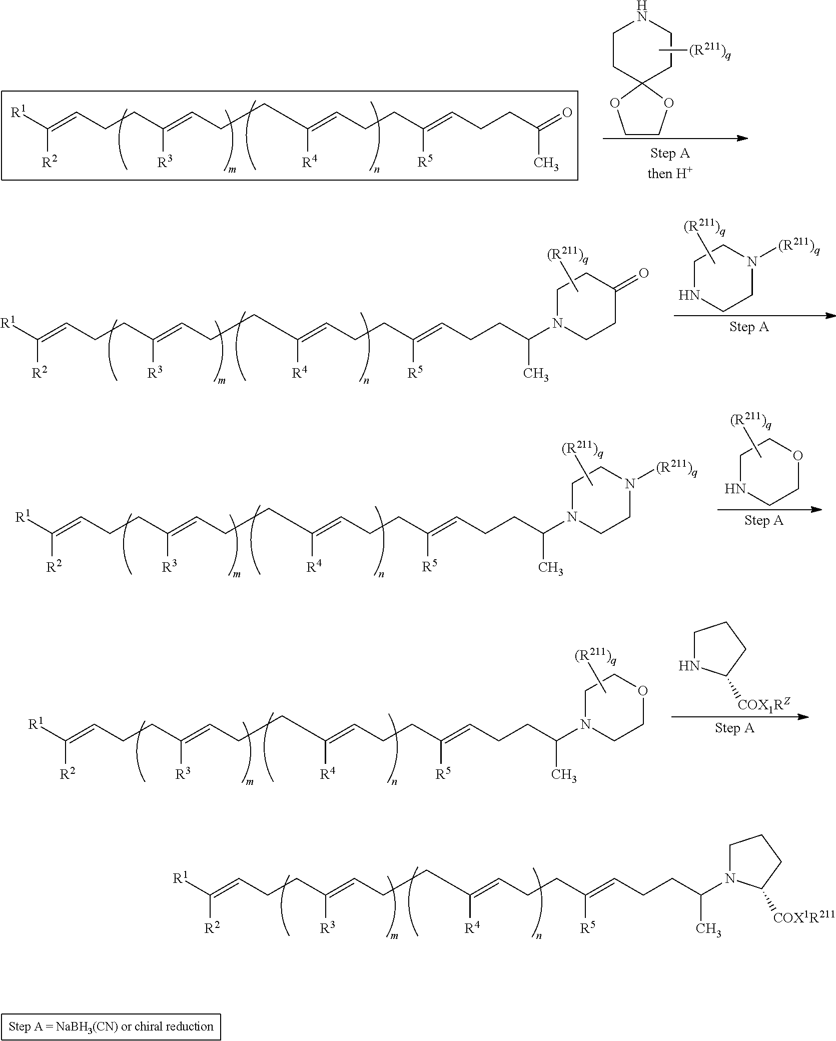

wherein the variables are as defined herein.

Provided below are representative synthetic routes to compounds having a bond for an L group:

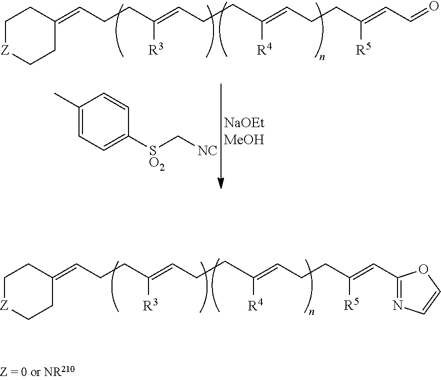

wherein the variables are as defined herein.

Provided below are representative synthetic routes to compounds having a bond for an L group:

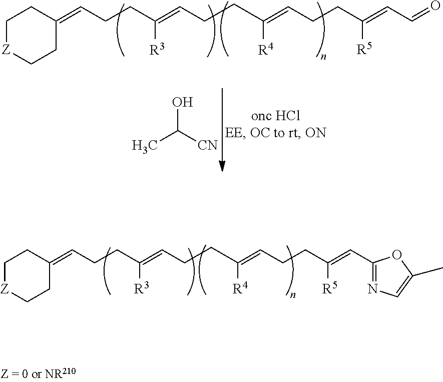

wherein the variables are as defined herein.

Provided below are representative synthetic routes to compounds having a bond for an L group:

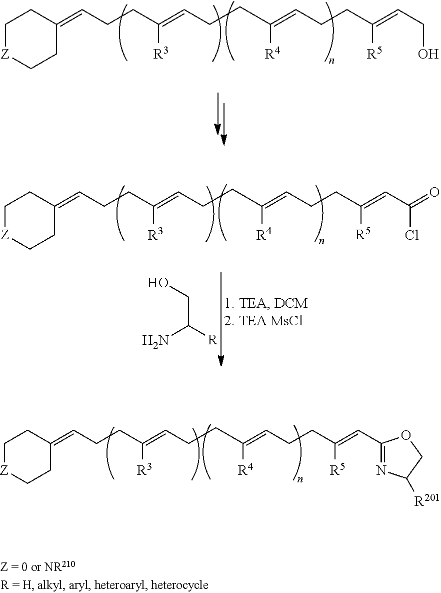

wherein the variables are as defined herein.

Provided below are representative synthetic routes to compounds having a bond for an L group:

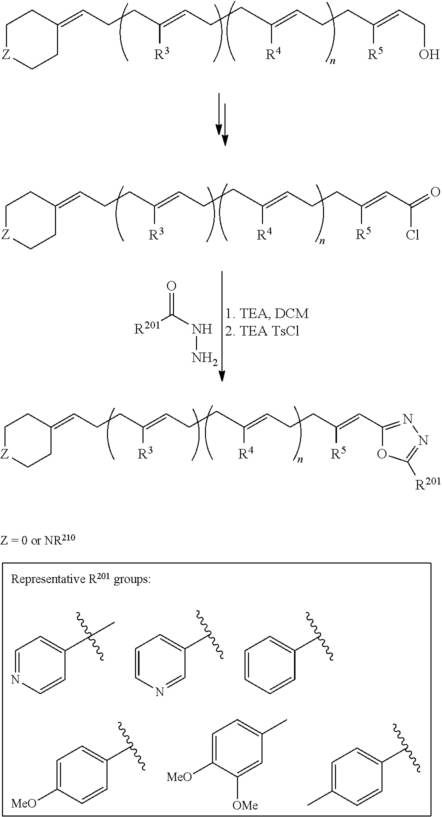

wherein the variables are as defined herein.

Provided below are representative synthetic routes to compounds having a bond for an L group:

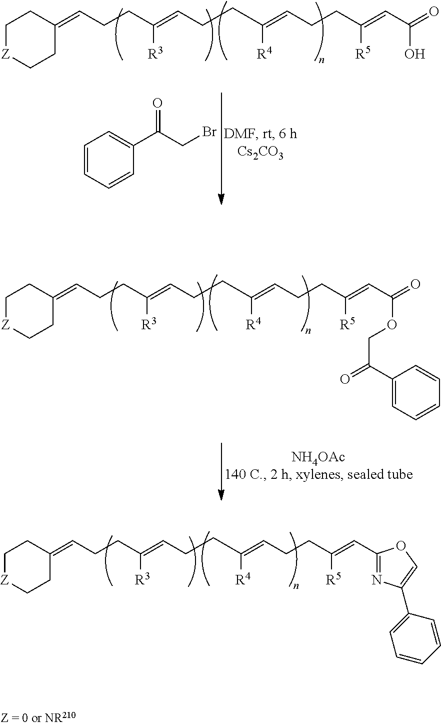

wherein the variables are as defined herein.

Provided below are representative synthetic routes to compounds having a bond for an L group:

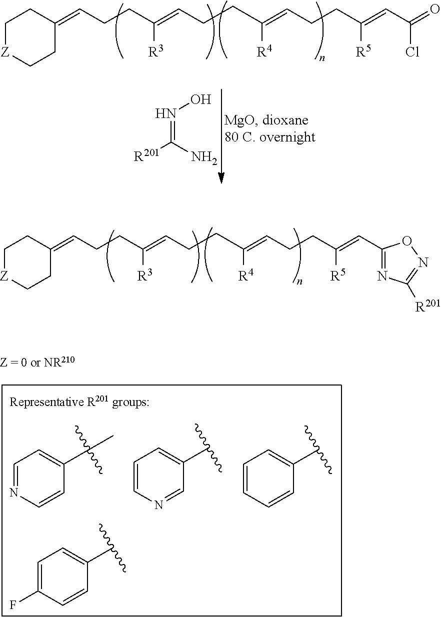

wherein the variables are as defined herein.

Provided below are representative synthetic routes to compounds having a bond for an L group.

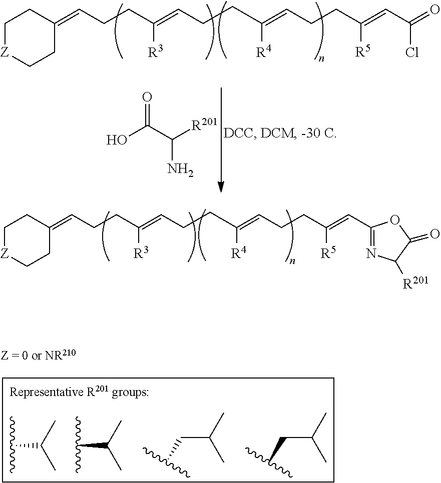

wherein the variables are as defined herein.

Some compounds provided and/or utilized in this invention are synthesized, e.g., from a compound of formula (III-A):

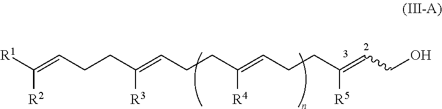

wherein n, R¹-R⁵ and [symbol] are defined as in Formula (I) above, following various well known methods upon substitution of reactants and/or altering reaction conditions as will be apparent to the skilled artisan upon reading this disclosure. The compound of Formula (III-A) is itself prepared by methods well known to a skilled artisan, for example, and without limitation, those described in PCT Pat. App. Pub. No. WO 2012/031028 and PCT Pat. App. No. PCT/US2012/027147 (each supra). An illustrative and non-limiting method for synthesizing a compound of Formula (III-A), where n is 1, is schematically shown below.

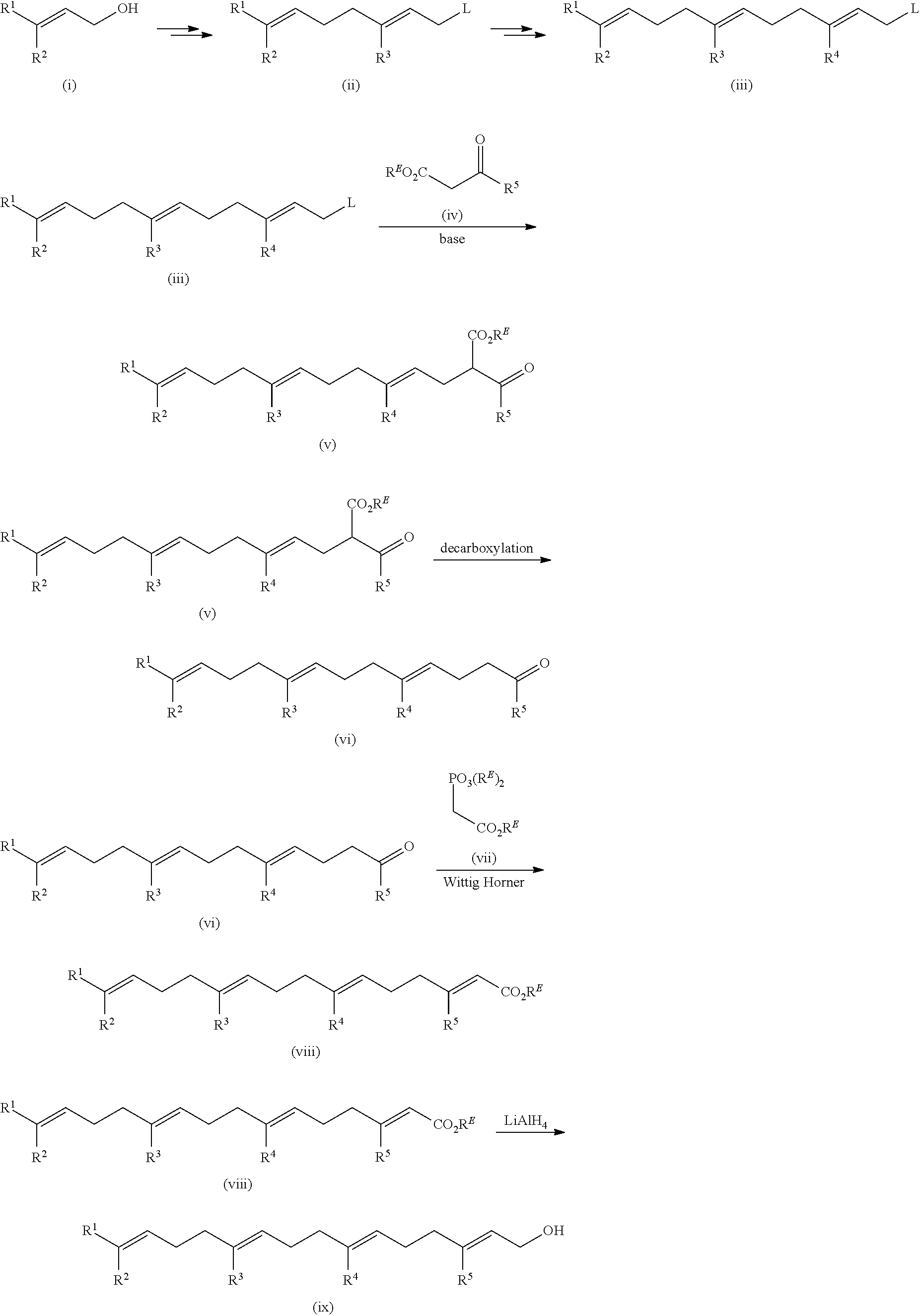

Starting compound (iii), which is synthesized from compound (i) by adding isoprene derivatives as described here, is alkylated with a beta keto ester (iv), in the presence of a base such as an alkoxide, to provide the corresponding beta-ketoester (v). Compound (v) upon alkaline hydrolysis followed by decarboxylation provides ketone (vi). Keto compound (vi) is converted, following a Wittig Horner reaction with compound (vii), to the conjugated ester (viii). Compound (viii) is reduced, for example with LiAlH₄, to provide alcohol (ix).

As will be apparent to the skilled artisan, a compound of Formula (III), where n is 2, is synthesized by repeating the reaction sequence of alkylation with a beta-keto ester, hydrolysis, decarboxylation, Wittig-Horner olefination, and LiAlH₄ reduction.

Certain illustrative and non-limiting synthesis of compounds provided and/or utilized in this invention are schematically shown below. Compounds where Q¹ is —(C═S)— or —SO₂— are synthesized by substituting the carbonyl group of the reactants employed, as will be apparent to the skilled artisan.

R⁶ in the schemes below may also correspond to R³⁰ and R⁵¹ as defined herein. R⁷ in the schemes below may also correspond to R²⁶, R³¹ and R⁵² as defined herein. R⁸ in the schemes below may also correspond to R²⁷, R³² and R⁵³ as defined herein. R⁹ in the schemes below may also correspond to R²⁸, R³³ and R⁵⁴ as defined herein. R¹³ in the schemes below may also correspond to R⁵⁸ as defined herein. R¹⁴ in the schemes below may also correspond to R⁵⁹ as defined herein. R¹⁵ in the schemes below may also correspond to R⁶⁹ as defined herein. R¹⁸ in the schemes below may also correspond to R²⁴, R³⁴ and R⁶³ as defined herein. R¹⁸ in the schemes below may also correspond to R²⁵, R³⁵ and R⁶⁴ as defined herein. L is a leaving group as known to one of ordinary skill in the art.

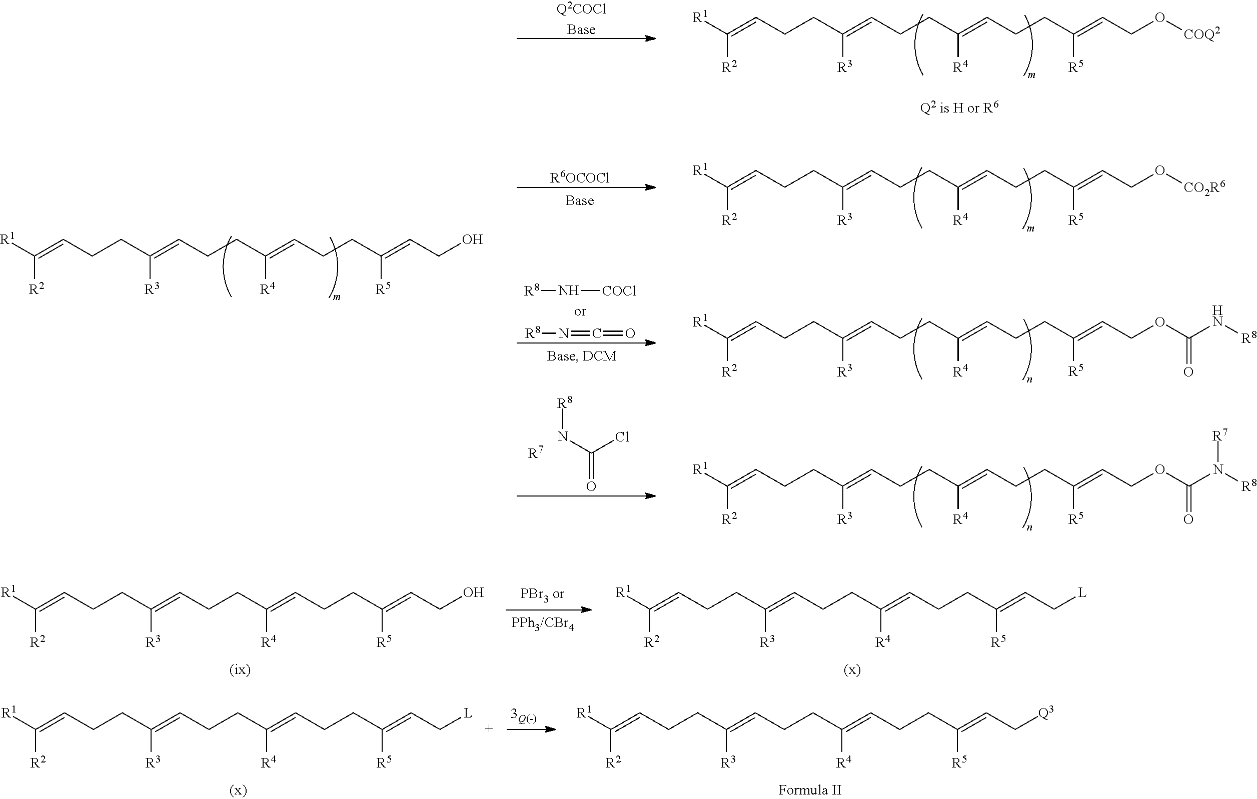

As shown above, R^{E} is alkyl and R¹ and R² is as defined herein above, and is preferably a heterocycle.

Compound (ix) with alcohol functionality is an intermediate useful for preparing the compounds provided and/or utilized in this invention. Compound (x), where L is an R⁵SO₂— group is made by reacting compound (ix) with R^{s}SO₂Cl in the presence of a base. The transformation of compound (iii) to compound (x) illustrates methods of adding isoprene derivatives to a compound, which methods are suitable to make compound (iii) from compound (i). Intermediate (ix) containing various R¹-R⁵ substituents are prepared according to this scheme as exemplified herein below. The transformation of compound (iii) to compound (x) illustrates methods of adding isoprene derivatives to a compound, which methods are suitable to make compound (iii) from compound (i).

The intermediates prepared above are converted to the compounds provided and/or utilized in this invention as schematically illustrated below:

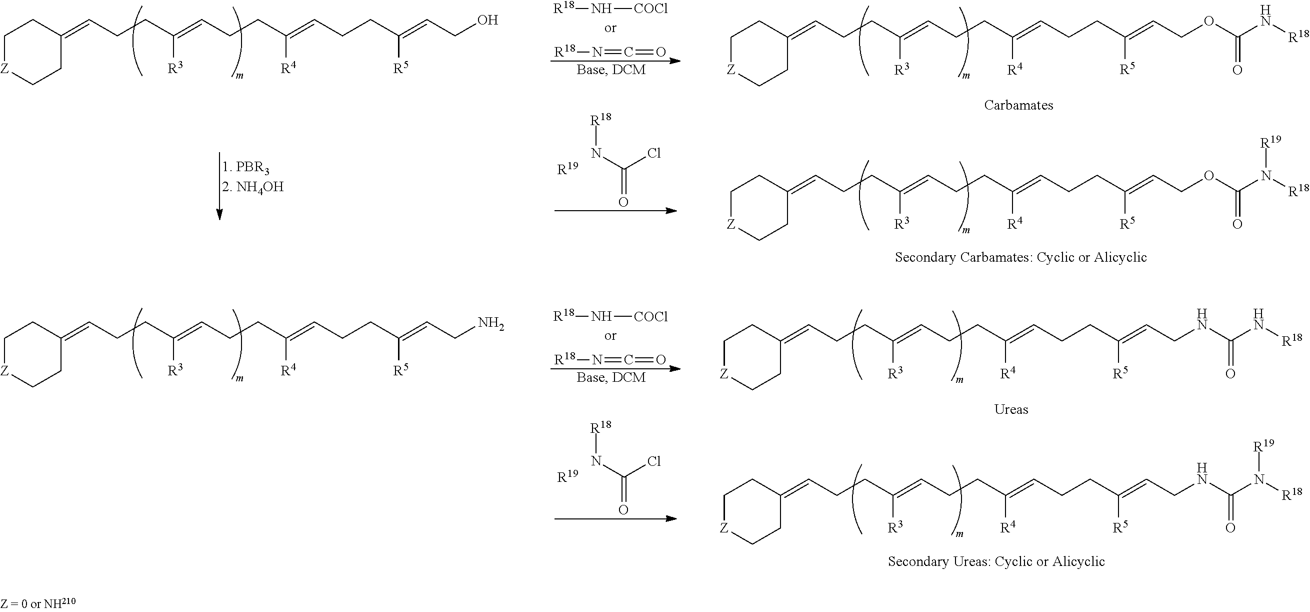

As used herein, for example, and without limitation, m is 0 or 1 and R¹-R⁵ are as defined herein, and are preferably alkyl, or more preferably methyl. Intermediate (ixa), prepared according to the scheme herein above, is converted to amino intermediate (ixb) via the corresponding bromide. Intermediates (ixa) and (ixb) are converted to the compounds provided and/or utilized in this invention by reacting with suitable isocyanates or carbamoyl chlorides, which are prepared by art known methods. The thiocarbamates and thioureas of this invention are prepared according to the methods described above and replacing the isocyanates or the carbamoyl chlorides with isothiocyanates (R¹⁸—N═C═S) or thiocarbamoyl chlorides (R¹⁸—NH—C(═S)Cl or R¹⁸R¹⁹N—C(═S)Cl). These and other compounds provided and/or utilized in this invention are also prepared by art known methods, which may require optional modifications as will be apparent to the skilled artisan upon reading this disclosure. Intermediates for synthesizing compounds provided and/or utilized in this invention containing various R¹-R⁵ substituents are illustrated in the examples section and/or are well known to the skilled artisan.

Certain GGA derivatives provided and/or utilized herein are synthesized as schematically shown below.

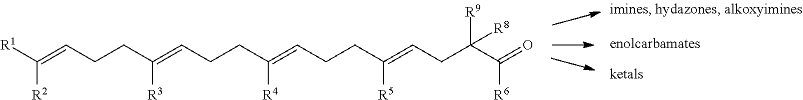

Certain compounds provided and/or utilized herein are obtained by reacting compound (x) with the anion Q(−), which can be generated by reacting the compound QH with a base. Suitable nonlimiting examples of bases include hydroxide, hydride, amides, alkoxides, and the like. Various compounds provided and/or utilized in this invention, wherein the carbonyl group is converted to an imine, a hydrazone, an alkoxyimine, an enolcarbamate, a ketal, and the like, are prepared following well known methods.

Other methods for making the compounds provided and/or utilized in this invention are schematically illustrated below:

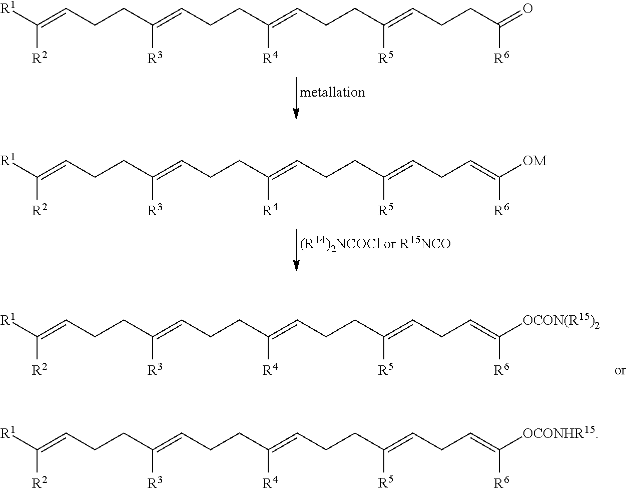

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine (R¹⁴)₂NH with phosgene or an equivalent reagent well known to the skilled artisan.

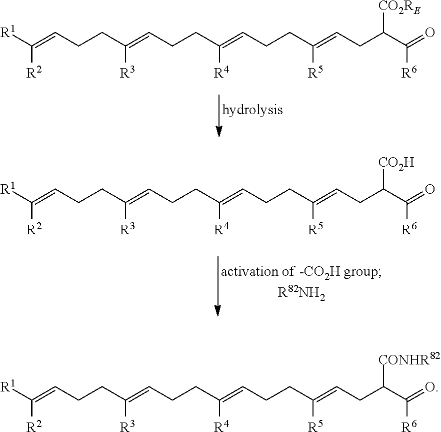

The beta keto ester is hydrolyzed while ensuring that the reaction conditions do not lead to decarboxylation. The acid is activated with various acid activating agent well known to the skilled artisan such as carbonyl diimodazole, or O-Benzotriazole-N,N,N′,N′-tetramethyl-uronium-hexafluoro-phosphate (HBTU) and reacted with the amine.

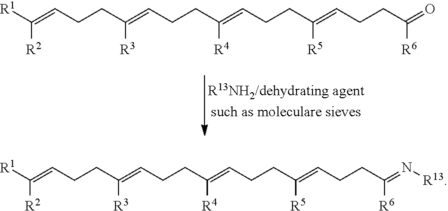

Various other compounds provided and/or utilized in this invention are prepared from the compounds made in the scheme above based on art known methods.

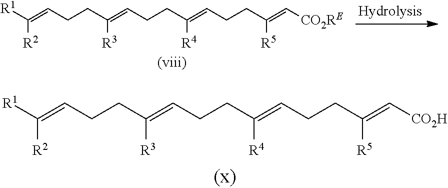

As shown above, R^{E} is alkyl.

The intermediates prepared above are converted to the compounds provided and/or utilized in this invention as schematically illustrated below:

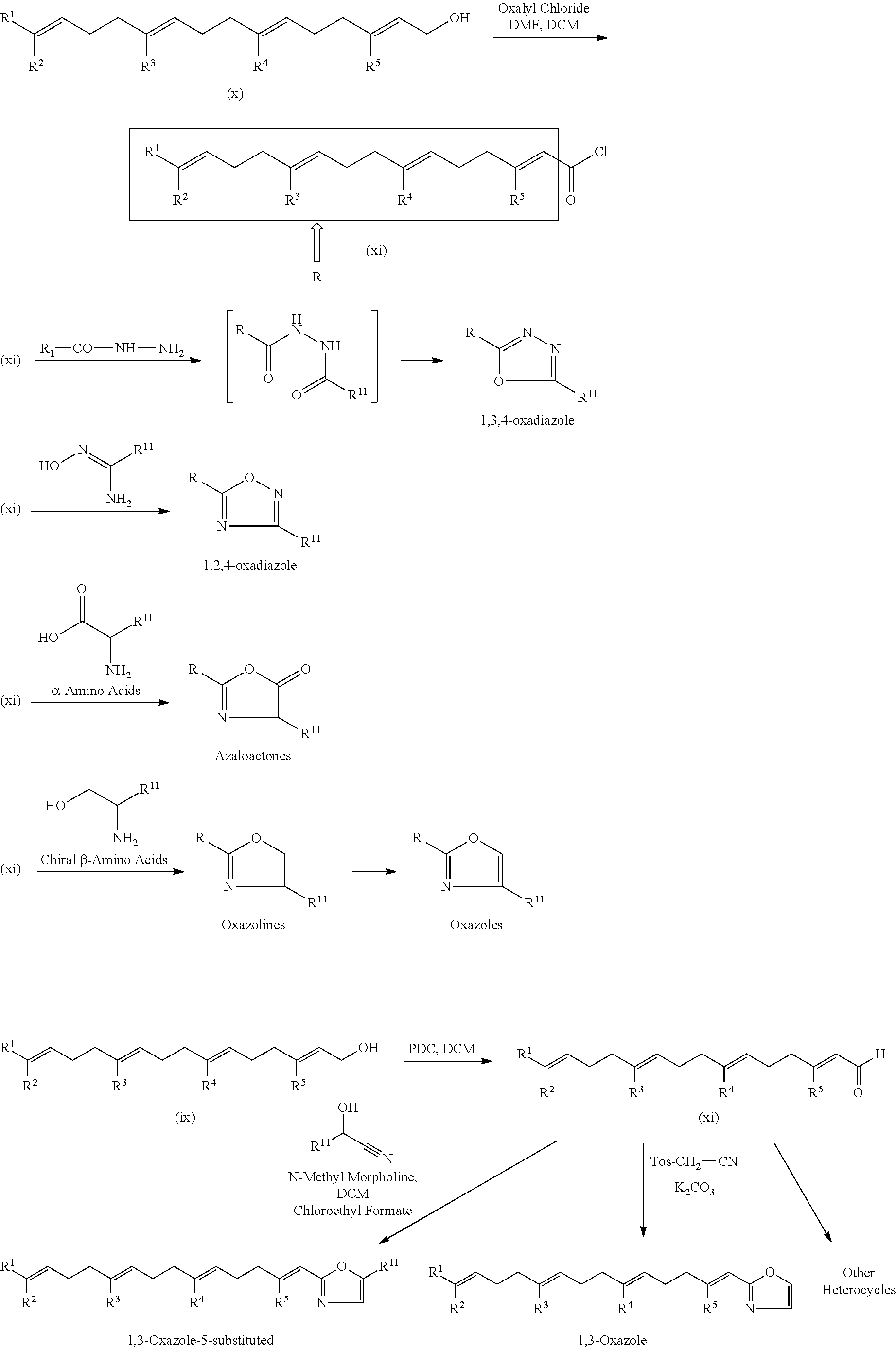

Compound (viii) is hydrolyzed to the carboxylic acid (x), which is then converted to the acid chloride (xi). Compound (xi) is reacted with a suitable nucleophile such as a hydrazide, a hydroxylamine, an amino alcohol, or an amino acid, and the intermediate dehydrated to provide a compound of Formula (IV). Alternatively, the allylic alcohol (ix) is oxidized to the aldehyde (xi), which is then reacted with a cyanohydrin or cyanotosylmethane to provide further compounds provided and/or utilized in this invention.

GGA derivatives provided and/or utilized in this invention can also be synthesized employing art known methods and those disclosed here by alkene-aryl, alkene-heteroaryl, or alkene-akene couplings such as Heck, Stille, or Suzuki coupling. Such methods can use (vi) to prepare intermediate (xii) that can undergo Heck, Stille, or Suzuki coupling under conditions well known to the skilled artisan to provide compounds provided and/or utilized in this invention.

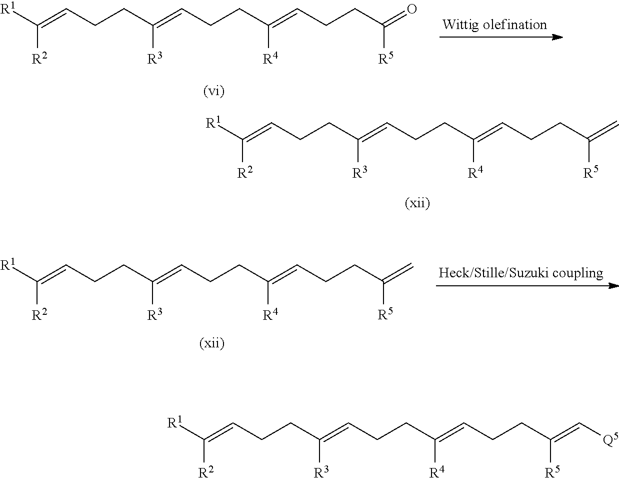

Higher and lower isoprenyl homologs of intermediates (x), (xi), and (xii), which are prepared following the methods disclosed here, can be similarly employed to prepare other compounds provided and/or utilized in this invention.

Compounds provided and/or utilized in this invention are also prepared as shown below

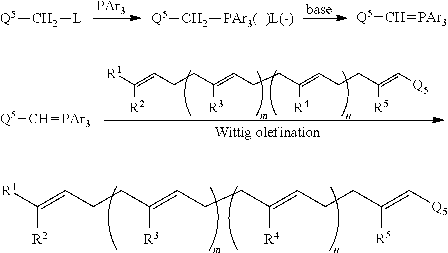

In the scheme above, R¹ and R² is as defined herein above, and is preferably a heterocycle.

L is a leaving group and Q₅ are as defined herein, Ar is a preferably an aryl group such as phenyl, the base employed is an alkoxide such as tertiarybutoxide, a hydride, or an alkyl lithium such as n-butyl lithium. Methods of carrying out the steps shown above are well known to the skilled artisan, as are conditions, reagents, solvents, and/or additives useful for performing the reactions and obtaining the compound of Formula (IV) in the desired stereochemistry.

Other methods for making the compounds provided and/or utilized in this invention are schematically illustrated below:

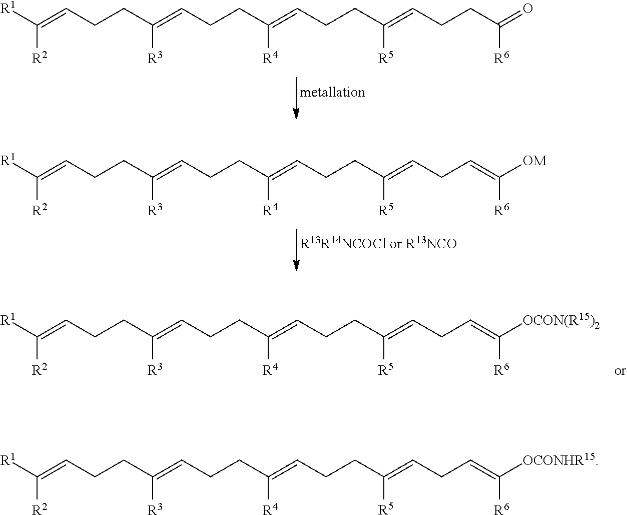

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine R¹³R¹⁴NH with phosgene or an equivalent reagent well known to the skilled artisan.

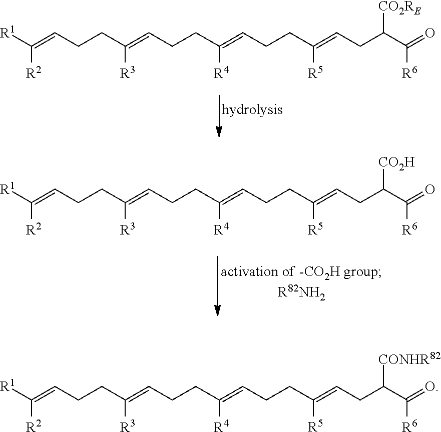

The beta keto ester is hydrolyzed while ensuring that the reaction conditions do not lead to decarboxylation. The acid is activated with various acid activating agent well known to the skilled artisan such as carbonyl diimodazole, or O-Benzotriazole-N,N,N′,N′-tetramethyl-uronium-hexafluoro-phosphate (HBTU) and reacted with the amine. Certain other methods of preparing the conjugates are shown below.

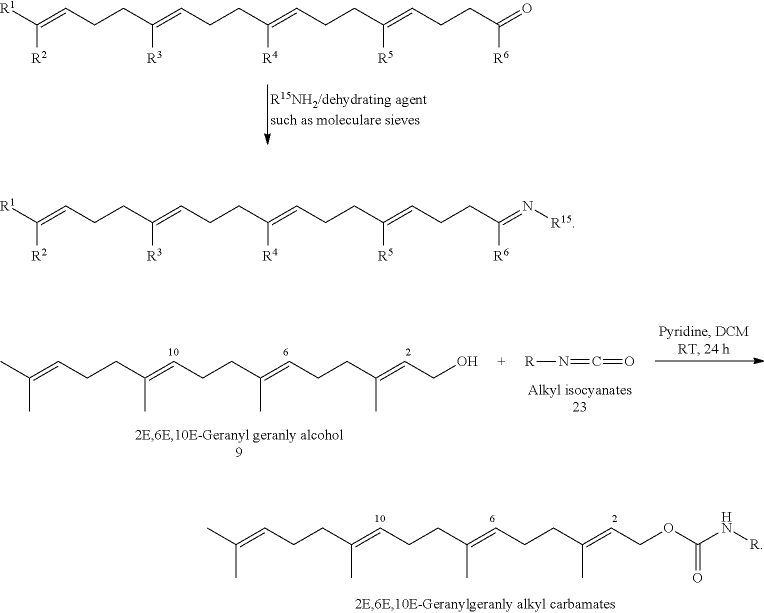

As shown above, R is a memantine or a riluzole residue, and R¹ and R² together with the carbon atom they are attached form a 5-6 membered heterocycle containing up to 3 ring heteroatoms, wherein the heteroatom is selected from the group consisting of O, N, S, and oxidized forms of N and S, and further wherein the heterocyclyl ring is optionally substituted.

In the schemes above, unless otherwise mentioned, R¹ and R² is as defined herein above, and is preferably a heterocycle.

### 6. UTILITY

GGA is a known anti-ulcer drug used commercially and in clinical situations. GGA has also been shown to exert cytoprotective effects on a variety of organs, such as the eye, brain, and heart (See for example Ishii Y., et al., Invest Ophthalmol V is Sci 2003; 44:1982-92; Tanito M, et al., J Neurosci 2005; 25:2396-404; Fujiki M, et al., J Neurotrauma 2006; 23:1164-78; Yasuda H, et al., Brain Res 2005; 1032:176-82; Ooie T, et al., Circulation 2001; 104:1837-43; and Suzuki S, et al., Kidney Int 2005; 67:2210-20).

In certain situations, the concentration of GGA required to exert a cytoprotective effect is an excessive amount of more than 600 mg per kg per day (Katsuno et al., Proc. Natl. Acad. Sci. USA 2003, 100, 2409-2414). The trans-isomer of GGA has been shown to be more efficacious at lower concentrations than a composition containing from 1:2 to 1:3 cis:trans mixture of GGA, and a composition of the cis-isomer of GGA alone. Therefore, the trans-isomer of GGA is useful for exerting cytoprotective effects on cells at a lower concentration than the cis-isomer or the 1:2 to 1:3 mixture of cis and trans isomers. Surprisingly, increasing amounts of the cis-isomer was found to antagonize the activity of the trans-isomer, as exemplified below.

It is contemplated that the isomeric mixture of GGA and/or compositions containing the 5-trans isomer of GGA can be used to inhibit neural death and increase neural activity in a mammal suffering from a neural disease, wherein the etiology of said neural disease comprises formation of protein aggregates which are pathogenic to neurons which method comprises administering to said mammal an amount of GGA which will inhibit neural death and increase neural activity, or impede the progression of the neural disease. As it relates to the isomeric mixture of GGA, this method is not intended to inhibit or reduce the negative effect of a neural disease in which the pathogenic protein aggregates are intranuclear or diseases in which the protein aggregation is related to SBMA.

Negative effects of neural diseases that are inhibited or reduced by GGA and the 5-trans isomer of GGA according to this invention include but are not limited to Alzheimer's disease, Parkinson's disease, multiple sclerosis, prion diseases such as Kuru, Creutzfeltdt-Jakob disease, Fatal familial insomnia, and Gerstmann-Straussler-Scheinker syndrome, amyotrophic lateral sclerosis, or damage to the spinal cord. GGA and the 5-trans isomer of GGA are also contemplated to prevent neural death during epileptic seizure.

As will be apparent upon reading this disclosure, certain GGA derivatives provided herein are useful as synthetic intermediates in the synthesis and/or manufacture of other GGA derivatives.

### 7. ASSAYS

The isolated cis- and trans-compounds described herein are also useful in assays which access a compound having putative cytoprotective effects. In particular, in such assays, the cis-isomer of GGA will behave as baseline or negative control and the trans-isomer as a positive control. The putative compound is tested in the assay described variously herein and its activity correlated against the cis- and trans-isomers. Compounds exhibiting activity similar to or exceeding that of the trans-isomer would be considered to be active compounds. Compounds providing activity similar to the cis-isomer would be considered to be inactive compounds. Accordingly, the cis-isomer finds utility as a negative control in the assay.

### 8. EXAMPLES OF THE INVENTION

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

In the examples below as well as throughout the application, the following abbreviations have the following meanings. If not defined, the terms have their generally accepted meanings.
° C.=degrees Celsius
PBr₃=phosphorus tribromide
EE=ethyl ether
EtOH=Ethanol
NaOEt=sodium ethoxide
Oet=Ethoxide
N=Normal
KOH=potassium hydroxide
aq=aqueous
h=hour(s)
RT=Room temperature
LAH=lithium aluminum hydride
THF=Tetrahydrofuran
min=minute(s)
Et=Ethyl
MeOH=Methanol
NaH=sodium hydride
ON=Overnight
E or (E)=Trans
Z or (Z)=Cis
TLC=thin layer chromatography
GGA=geranylgeranyl acetone
μL=Microliter
mL=Milliliter
HPC=hydroxypropyl cellulose
DI=Deionized
Av=Average
p-TsOH=p-toluenesulfonic acid
Ph₃P=Triphenylphosphine
Br-=bromide ion
CBr₄=Tetrabromomethane
LC-MS=Liquid chromatography-mass spectrometry
PEG-200 polyethylene glycol
KHMDA=potassium hexamethylenediamine
ACN=Acetonitrile

The starting materials for the reactions described below are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1 15 (John Wiley and Sons, 1991), Rodd's Chemistry of Carbon Compounds, Volumes 1 5 and Supplementals (Elsevier Science Publishers, 1989), Organic Reactions, Volumes 1 40 (John Wiley and Sons, 1991), March's Advanced Organic Chemistry, (John Wiley and Sons, 4.sup.th Edition), and Larock's Comprehensive Organic Transformations (VCH Publishers Inc., 1989).

### Example 1

### 5E,9E,13E-Geranylgeranyl Acetone Synthesis

### Synthesis of 5-trans-Isomer: 5E,9E,13E-Geranylgeranyl acetone 1

The synthesis of 5-trans isomer: 5E,9E,13E-geranylgeranyl acetone 1 can be achieved as per outlined in the scheme below.

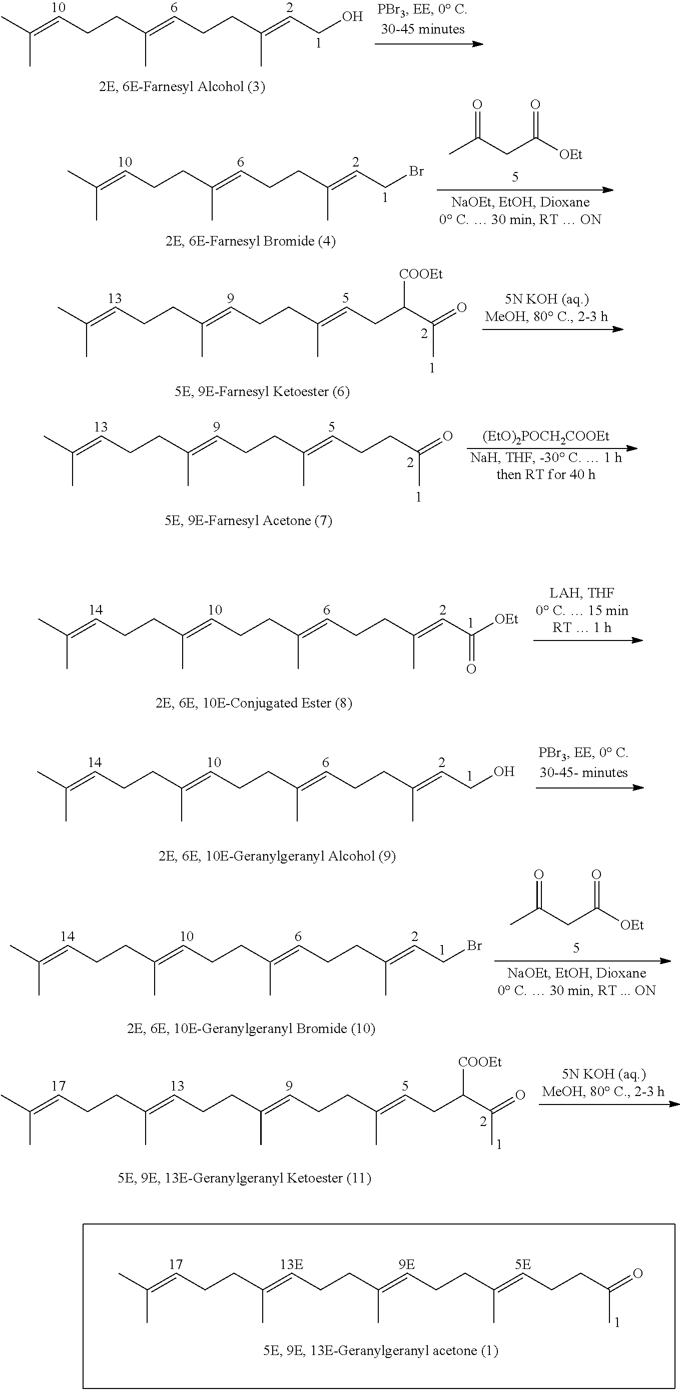

The 2E,6E-farnesyl alcohol 3 (where the geometry at C2 and C6 positions is already fixed as trans- or E) was designed and used as a commercially available starting material for the synthesis of 5E,9E,13E-geranylgeranyl acetone 1. The alcohol function of 2E,6E-farnesyl alcohol 3 was converted to the corresponding bromide 4 by the treatment of phosphorus tribromide (PBr₃) in ethyl ether (EE) or with Ph₃P and CBr₄ in acetonitrile (ACN) at 0° C. The resulting bromide was then reacted with carbanion (derived from the reaction of ethyl acetoacetate 5 and sodium ethoxide) to yield the desired 5E,9E-farnesyl ketoester 6. The homologated ketoester 6 after hydrolysis and decarboxylation using aqueous 5N KOH yielded the expected 5E,9E-farnesyl acetone 7. A one pot conversion of bromide 4 to the corresponding farnesyl acetone 7 can be possible without isolating intermediate ketoester 6.

In order to generate the trans-orientation of olefin at C2 of conjugated olefin 8 in a key step, the reaction of 5E,9E-farnesyl acetone 7 with carbanion [derived from the reaction of (EtO)₂PO—CH₂—COOEt and sodium hydride (NaH)] at −30° C. was conducted to obtain the desired 2E,6E,10E-conjugated ester 8. The formation of the product 8 with the exclusive trans (E) geometry was observed when the reaction was conducted at −30° C. or temperature below −30° C., where all the three olefins are set in a trans (E) orientation (Ref.: Kato et al., J. Org. Chem. 1980, 45, 1126-1130 and Wiemer et al., Organic Letters, 2005, 7(22), 4803-4806). The minor cis-(Z)-isomer was eliminated/separated from the trans-(E)-isomer 8 by a careful silica gel column chromatographic purification. However, it was also noted that the formation the corresponding cis-isomer (Z) was increased when the reaction was conducted at 0° C. or at higher temperature. It was also noted that the mixture of cis (2Z)- and trans (2E)-isomer of 8 can be separated by a very careful column chromatographic separation.

The resulting 2E-conjugated ester 8 was reduced to the corresponding 2E-alcohol 9 by means of a lithium aluminum hydride (LAH) treatment, which was then converted into the corresponding 2E,6E,10E-geranylgeranyl bromide 10 by means of phosphorus tribromide (PBr₃) treatment in ethyl ether (EE) or with Ph₃P and CBr₄ in acetonitrile (ACN) at 0° C. Furthermore, the interaction of carbanion (derived from ethyl acetoacetate 5 and sodium ethoxide) with the bromide 10 at 0° C. afforded the desired 2E,6E,10E-geranylgeranyl ketoester 11, a precursor needed for 5E,9E,13E-geranylgeranyl acetone 1. The subsequent ester hydrolysis and decarboxylation of ketoester 11 using aq. 5N KOH at 80° C. yielded the requisite 5E,9E,13E-geranylgeranyl acetone 1. TLC Rf: 0.28 (5% Ethyl Acetate in Hexanes); LC Retention time: 16.68 min; MS (m/e): 313 [M-18+H]+, 331 [MH]+, 353 [M+K].

### Example 2

### 5-Z,9E,13E-Geranylgeranyl Acetone Synthesis

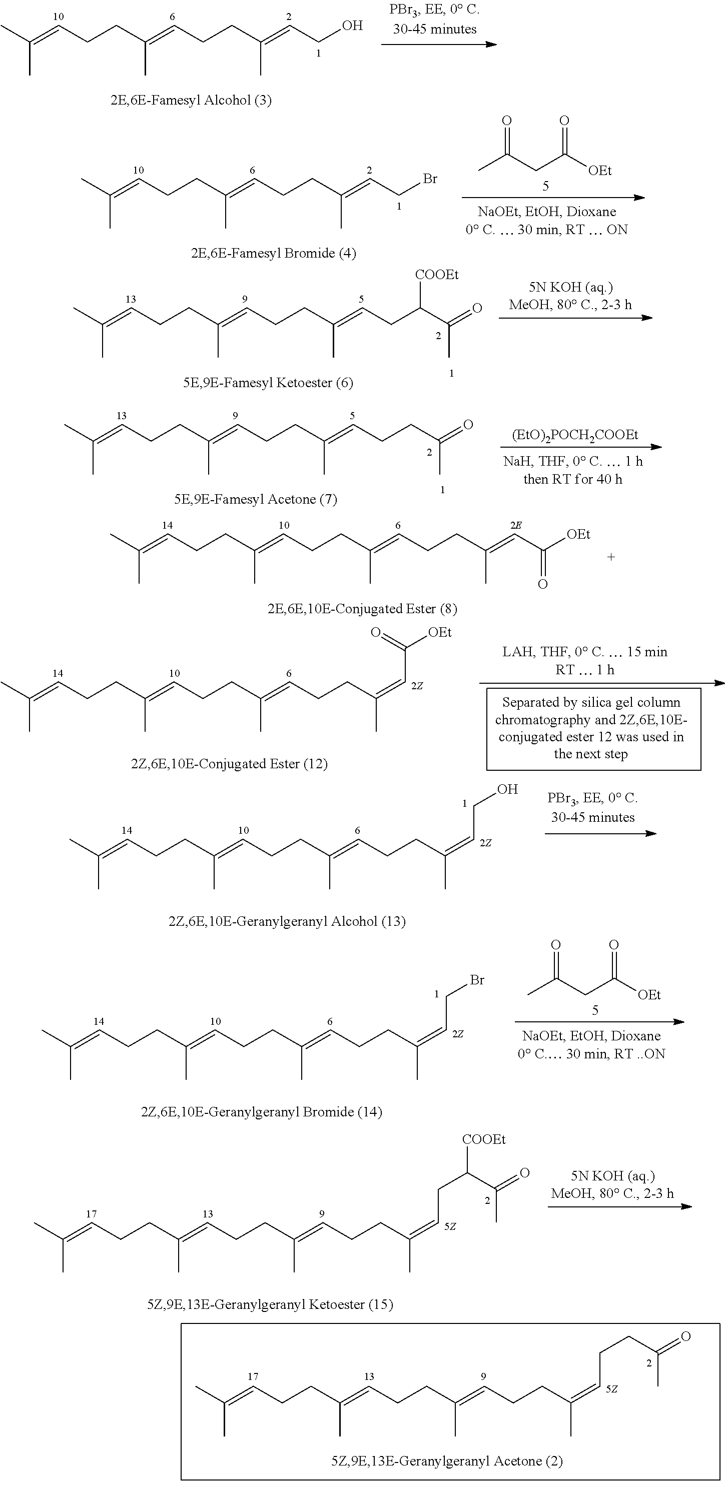

The 2E,6E-farnesyl alcohol 3 (where the geometry at C2 and C6 positions is already fixed as trans- or E) was used as a commercially available starting material for the synthesis of 5Z,9E,13E-geranylgeranyl acetone 2. The reaction of farnesyl alcohol 3 with phosphorus tribromide (PBr₃) in ethyl ether (EE) or with Ph₃P and CBr₄ in acetonitrile (ACN) at 0° C. afforded the requisite bromide 4, which was then reacted with carbanion (derived from the reaction of ethyl acetoacetate 5 and sodium ethoxide) to yield the desired 5E,9E-farnesyl ketoester 6. The homologated ketoester 6 after hydrolysis and decarboxylation using aqueous 5N KOH yielded the expected 5E,9E-farnesyl acetone 7, one of the key intermediate for the synthesis of 5E,9E,13E-geranylgeranyl acetone 1 and 5Z,9E,13E-geranylgeranyl acetone 2.

With a view to obtain product with cis-geometry at C2 with the conjugated olefin 12, the reaction of 5E,9E-farnesyl acetone 7 with carbanion [derived from the reaction of (EtO)₂PO—CH₂—COOEt and sodium hydride (NaH)] at 0° C. was conducted. This reaction afforded a mixture of 2E,6E,10E-conjugated ester 8 and 2Z,6E,10E-conjugated ester 12, from which the C2-cis (Z)-isomer 12 was separated by a repeated and careful silica gel column chromatography (Ref. Kato et al., J. Org. Chem., 1980, 45, 1126-1130).

The resulting 2Z-conjugated ester 12 was converted into the corresponding 2Z-alcohol 13 by means of a lithium aluminum hydride (LAH) treatment. The 2Z-alcohol 13 was transformed into the corresponding 2Z,6E,10E-geranylgeranyl bromide 14 by using phosphorus tribromide (PBr₃) treatment in ethyl ether (EE) or with Ph₃P and CBr₄ acetonitrile (ACN) at 0° C., and then reacted with carbanion (derived from ethyl acetoacetate 5 and sodium ethoxide) at 0° C. afforded the desired 2Z,6E,10E-geranylgeranyl ketoester 15, a precursor needed for 5Z,9E,13E-geranylgeranyl acetone 2. The subsequent ester hydrolysis and decarboxylation of ketoester 15 using aq. 5N KOH at 80° C. yielded the requisite 5Z,9E,13E-geranylgeranyl acetone 2.

### Example 3

### 5Z,9E,13E-Geranylgeranyl Acetone Synthesis

### Alternative synthesis of 5-cis Isomer: 5Z,9E,13E-Geranylgeranyl acetone 2

The alternative synthesis of 5Z,9E,13E-geranylgeranyl acetone 2 can be achieved as shown in the scheme below.

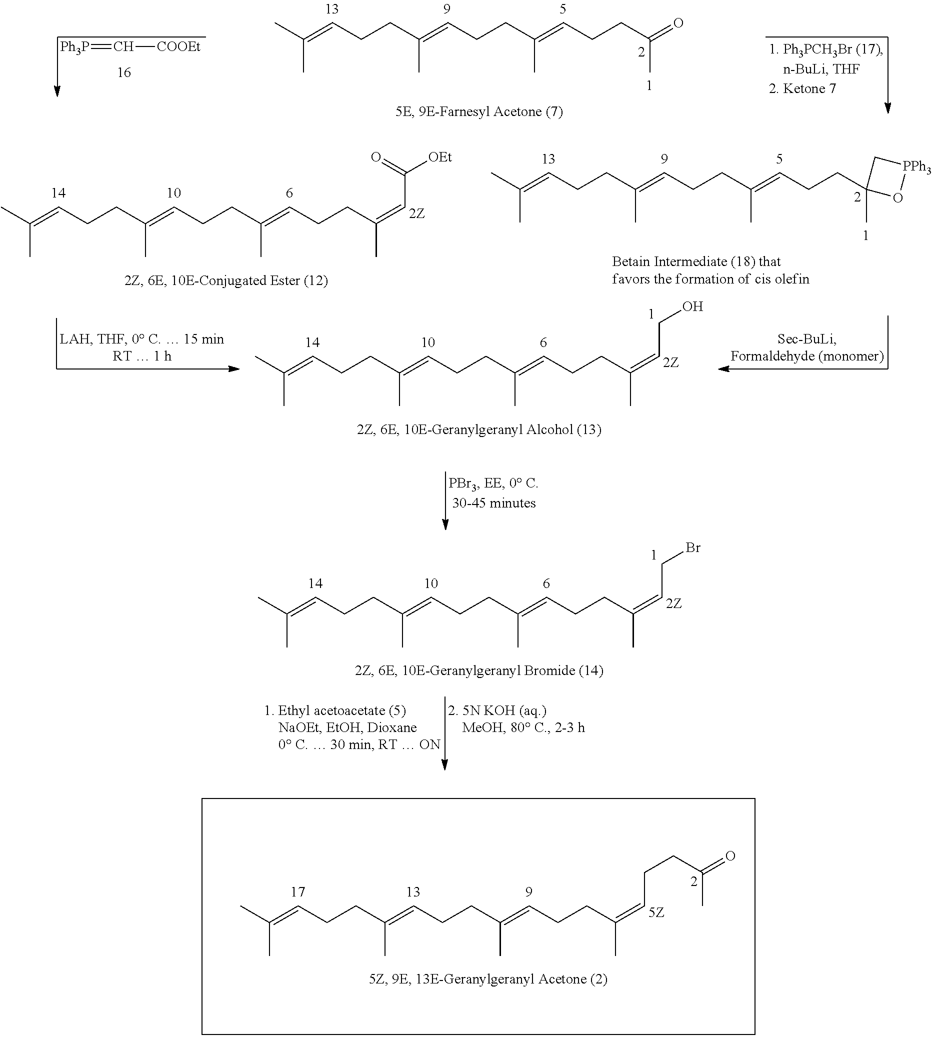

The use of 5E,9E-farnesyl acetone 7, as a key intermediate, can be used to generate additional double bond with cis-(Z)-orientation. In one approach, the reaction of 5E,9E-farnesyl acetone 7 with the witting reagent 16 can afford the conjugated ester 12 with cis-(Z)-geometry at C2 position. The subsequent reduction of ester 12 with lithium aluminum hydride (LAH) can generate the corresponding alcohol 13, which then can be converted into the corresponding bromide 14. The conversion of bromide 14 to the ketoester 15 followed by hydrolysis and decarboxylation can afford the desired 5-cis (Z) isomer; 5Z,9E,13E-geranygeranyl acetone (2). In an alternative approach, the reaction of 5E,9E-farnesyl acetone 7 with triphenyl methylphosphonrane bromide 17 under a basic conditions followed by treatment with formaldehyde (monomeric) can afford the 2Z,6E10E-geranylgeranyl alcohol 13 with cis (Z)-orientation at C2 (Ref.: Wiemer et al., Organic Letters, 2005, 7(22), 4803-4806). The conversion of bromide 14 to the ketoester 15 followed by hydrolysis and decarboxylation can afford the desired 5-cis (Z)-isomer; 5Z,9E,13E-geranygeranyl acetone (2). TLC Rf: 0.32 (5% Ethyl Acetate in Hexanes); LC: Retention time: 17.18 min; MS (m/e): 313 [M-18+H]⁺, 331 [MH, very weak ionization]+, 339 [M-CH₂+Na], 353 [M+K].

All the intermediate products were purified by silica gel column chromatography and then used in the next step, except the bromides 4, 10 and 14. Due to the unstable nature of bromides 4, 10 and 14 towards silica gel column chromatography, these bromides were used in the next step without purification. Alternatively, all the intermediate products shown in the schemes 1, 2 and 3 are liquids and therefore can be separated and purified by a distillation process under appropriate levels of vacuum. All the intermediates and final products were characterized by LC-MS for mass along with the Thin Layer Chromatography (TLC) for Rf values.

### Example 4

### 5-Z,9E,13E-Geranylgeranyl Acetone Synthesis

### Alternative synthesis of 5-cis Isomer: 5Z,9E,13E-Geranylgeranyl acetone 2

The alternative synthesis of 5Z,9E,13E-geranylgeranyl acetone 2 can be achieved as shown in the scheme below

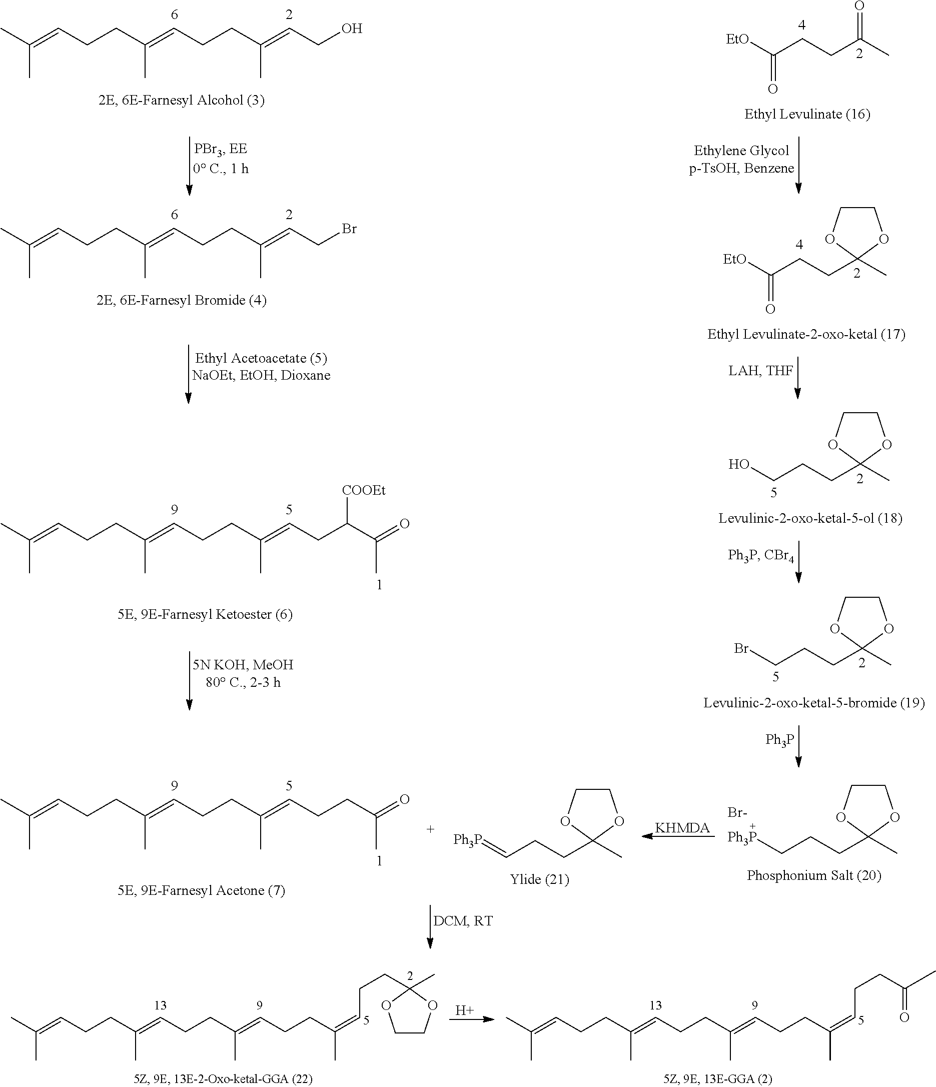

The convergent synthesis of 5Z,9E,13E-GGA 2 has been shown in the above scheme and is outlined as follows.

The 2E,6E-farnesyl alcohol 3 (where the geometry at C2 and C6 positions is already fixed as trans- or E) was used as a commercially available starting material for the synthesis of 5Z,9E,13E-geranylgeranyl acetone 2. The reaction of farnesyl alcohol 3 with phosphorus tribromide (PBr₃) in ethyl ether (EE) or with Ph₃P and CBr₄ in acetonitrile (ACN) at 0° C. afforded the requisite bromide 4, which was then reacted with carbanion (derived from the reaction of ethyl acetoacetate 5 and sodium ethoxide) to yield the desired 5E,9E-farnesyl ketoester 6. The homologated ketoester 6 after hydrolysis and decarboxylation using aqueous 5N KOH yielded the expected 5E,9E-farnesyl acetone 7, one of the key intermediate for the synthesis of 5E,9E,13E-geranylgeranyl acetone 1 and 5Z,9E,13E-geranylgeranyl acetone 2.

The other synthon, namely the ylide 21 can be synthesized from a commercially available starting material, ethyl levulinate 16, a sugar industry by-product. The ketalization of ethyl levulinate 16 using conventional conditions (ethylene glycol, p-TsOH, azeotropic reflux) can yield the desired 2-oxo-ketal 17, which then can be reduced using LAH in THF at 0° C. to the corresponding alcohol 18. Furthermore, the alcohol 18 then can be treated with Ph₃Br in diethyl ether at 0° C. to obtain the bromide 19, which then after treatment with Ph₃P can yield the phosphonium bromide salt 20. The bromide salt 20 upon treatment with mild alkali (1N NaOH) can furnish the desired ylide 21, required to complete the synthesis of 5Z-GGA 2.

With a view to obtain product with cis-geometry, the reaction of 5E,9E-farnesyl acetone 7 with the ylide 21 in DCM at RT can afford the desired 5Z-oxoketal 22 (Ref.: Ernest et al, Tetrahedron Lett. 1982, 23(2), 167-170). The protected oxo-function from 22 can be removed by means of a mild acid treatment to yield the expected 5Z,9E,13E-GGA 2.

### Example 5

### 5E,9E,13E-Geranylgeranyl Acetone Synthesis

### Alternative synthesis of 5-trans Isomer: 5E,9E,13E-Geranylgeranyl acetone 1

The alternative synthesis of 5E,9E,13E-geranylgeranyl acetone 1 can be achieved as shown in the scheme below.

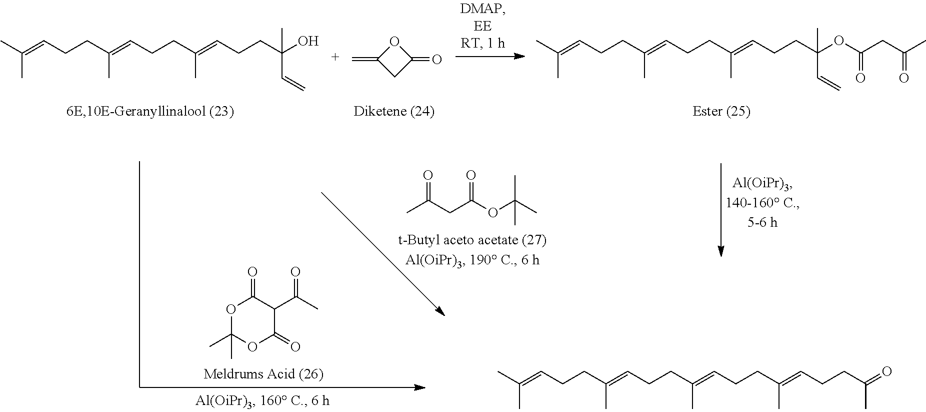

The 5E, 9E, 13E-geranyl geranyl acetone (1) can be prepared by reacting 6E-10E-geranyl linalool (23) with diketene (24) catalyzed by DMAP in ethyl ether to give the ester 25. The ester 25 in the Carroll rearrangement using Al(OiPr)₃ at elevated temperature can afford the desired 5E, 9E, 13E-geranyl geranyl acetone (1). In another approach, the GGA (1) can be prepared by treating geranyl linalool (23) with the Meldrum's acid 26 in the Carroll rearrangement using Al(OiPr)₃ at 160° C. Similarly, the use of tert-butyl acetoacetate (27) with geranyl linalool (23) in the Carroll rearrangement can also give the desired 5E,9E,13E-geranyl geranyl acetone (1).

### Example 6

### 5-Z,9E,13E-Geranylgeranyl Acetone Synthesis

The alternative synthesis of 5Z,9E,13E-geranylgeranyl acetone 2 can be achieved as shown in the scheme below

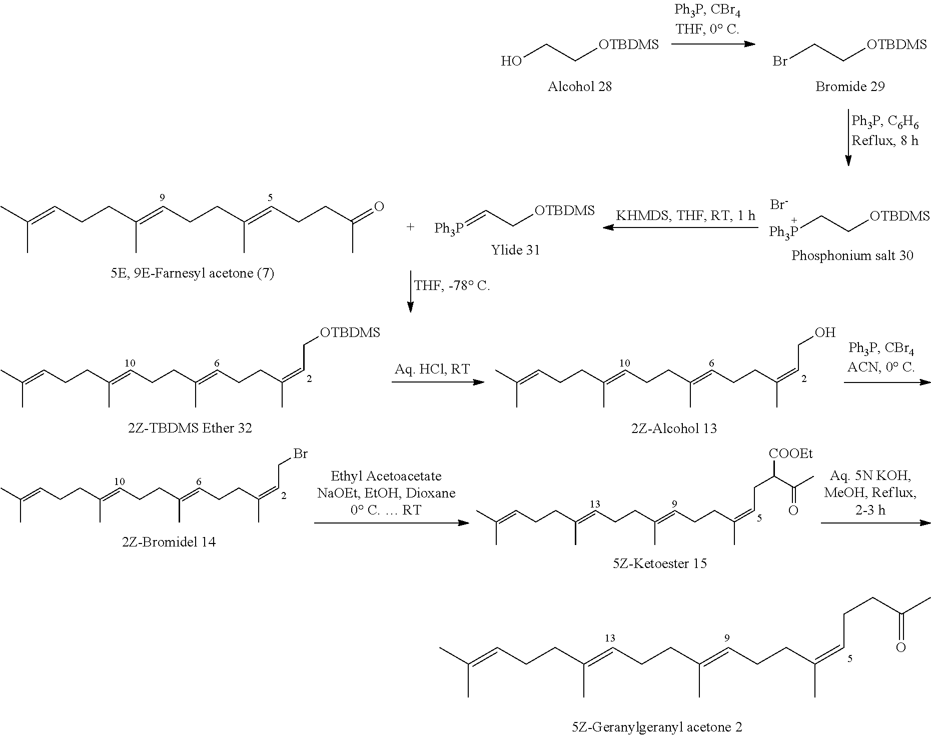

### Alternative synthesis of 5-cis Isomer: 5Z,9E,13E-Geranylgeranyl acetone 2

The 2E,6E-farnesyl alcohol 3 (where the geometry at C2 and C6 positions is already fixed as trans- or E) was used as a commercially available starting material for the synthesis of 5Z,9E,13E-geranylgeranyl acetone 2. The reaction of farnesyl alcohol 3 with phosphorus tribromide (PBr₃) in ethyl ether (EE) or with Ph₃P and CBr₄ in acetonitrile (ACN) at 0° C. afforded the requisite bromide 4, which was then reacted with carbanion (derived from the reaction of ethyl acetoacetate 5 and sodium ethoxide) to yield the desired 5E,9E-farnesyl ketoester 6. The homologated ketoester 6 after hydrolysis and decarboxylation using aqueous 5N KOH yielded the expected 5E,9E-farnesyl acetone 7, one of the key intermediate for the synthesis of 5E,9E,13E-geranylgeranyl acetone 1 and 5Z,9E,13E-geranylgeranyl acetone 2.

The ylide 31 synthesized from a commercially available mono-TBDMS protected ethylene glycol 28. The conversion of alcohol function of 28 by using Ph₃P and CBr₄ in acetonitrile can afford the corresponding bromide 29, which then can be used to make a phosphonium bromide salt 30 by treatment with Ph₃P at elevated temperature. The bromide salt 30 upon treatment with KHMDS in THF can afford the ylide 31, which then can be reacted in-situ with ketone 7 in a key step to establish cis geometry with the newly created double bond at C2 position and obtain the 2Z-TBDMS ether 32 (ref: Still et al, J. Org. Chem., 1980, 45, 4260-4262 and Donetti et al, Tetrahedron Lett. 1982, 23(21), 2219-2222). The deprotection of TBDMS with aqueous HCl to afford the corresponding alcohol 13 followed by conversion of alcohol to bromide using Ph₃P and CBr₄ can afford the desired bromide 14. The bromide 14 upon reaction with ethyl acetoacetate can give ketoester 15, which then upon hydrolysis followed by decarboxylation can yield the desired 5-Z-GGA (5-cis) 2.

From the foregoing it will be appreciated that, although specific embodiments of the invention have been described herein for purposes of illustration, various modifications may be made without deviating from the spirit and scope of the invention.

Throughout the description of this invention, reference is made to various patent applications and publications, each of which are herein incorporated by reference in their entirety.

What is claimed is:

1. A compound of Formula (I): 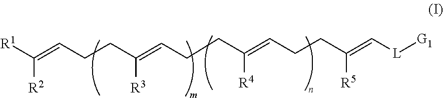
or pharmaceutically acceptable salt thereof, wherein
n is 0, 1 or 2;
m is 0 or 1;
L is a bond or C₁-C₆ alkylene;
G₁ is
—C(═O)H, —CO₂H or an ester or acyl halide thereof;
a 5-14 membered heteroaryl or heterocycle containing up to 6 ring heteroatoms, wherein the heteroatom is selected from the group consisting of O, N, S, and oxidized forms of N and S;
wherein the ring B, heteroaryl, or heterocyclyl is optionally substituted with 1-3 substituents selected from the group consisting of:
hydroxy, oxo, —N(R⁴⁰)₂, C₁-C₆ alkoxy group, C₁-C₆ alkyl group, C₃-C₁₀ cycloalkyl, —CO₂H or an C₁-C₆ alkyl ester or an C₁-C₆ alkyl amide thereof, wherein the cycloalkyl group is optionally substituted with 1-3 C₁-C₆ alkyl groups,
a 5-9 membered heteroaryl or heterocyclyl containing up to 3 ring heteroatoms, wherein the heteroaryl or heterocyclyl is optionally substituted with 1-3 hydroxy, —N(R⁴⁰)₂, or C₁-C₆ alkyl groups, and
benzyl or C₆-C₁₀ aryl, optionally substituted with 1-3 substituents selected from the group consisting of C₁-C₆ alkyl, C₁-C₆ alkoxy, hydroxy, and halo groups;
each R¹ and R² are independently C₁-C₆ alkyl, or R¹ and R² together with the carbon atom they are attached to form a C₄-C₇ cycloalkyl ring optionally substituted with 1-3 C₁-C₆ alkyl groups; or a 5-6 membered heterocycle containing up to 3 ring heteroatoms, wherein the heteroatom is selected from the group consisting of O, N, S, and oxidized forms of N and S, and further wherein the heterocyclyl ring is optionally substituted with 1-3 substituents selected from the group consisting of:
hydroxy, oxo, —N(R⁴⁰)₂, C₁-C₆ alkoxy, and C₁-C₆ alkyl,
each of R³, R⁴, and R⁵ independently are hydrogen or C₁-C₆ alkyl;
R⁴⁰ is hydrogen or C₁-C₆ alkyl; and
R²⁰⁰ is hydrogen, CO₂H or an C₁-C₆ alkyl ester thereof;
with the proviso that if L is a bond G₁ is not —C(═O)H, —CO₂H or an ester or acyl halide thereof; and
with the proviso that if L is a bond or —CH₂—, R¹ and R² are not C₁-C₆ alkyl; and
with the proviso that if L is a bond or —CH₂—, R¹ and R² do not combine with the carbon to which they are attached to form a C₄-C₇cycloalkyl ring.

2. The compound of claim 1, wherein the compound is of Formula (II): 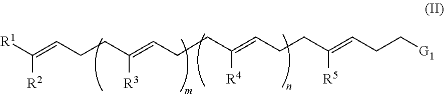
or pharmaceutically acceptable salt thereof.

3. The compound of claim 1, wherein the compound is of Formula (IIa): 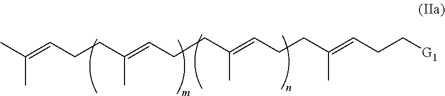
or pharmaceutically acceptable salt thereof.

4. The compound of claim 1, wherein the compound has a Formula selected from the group consisting of: 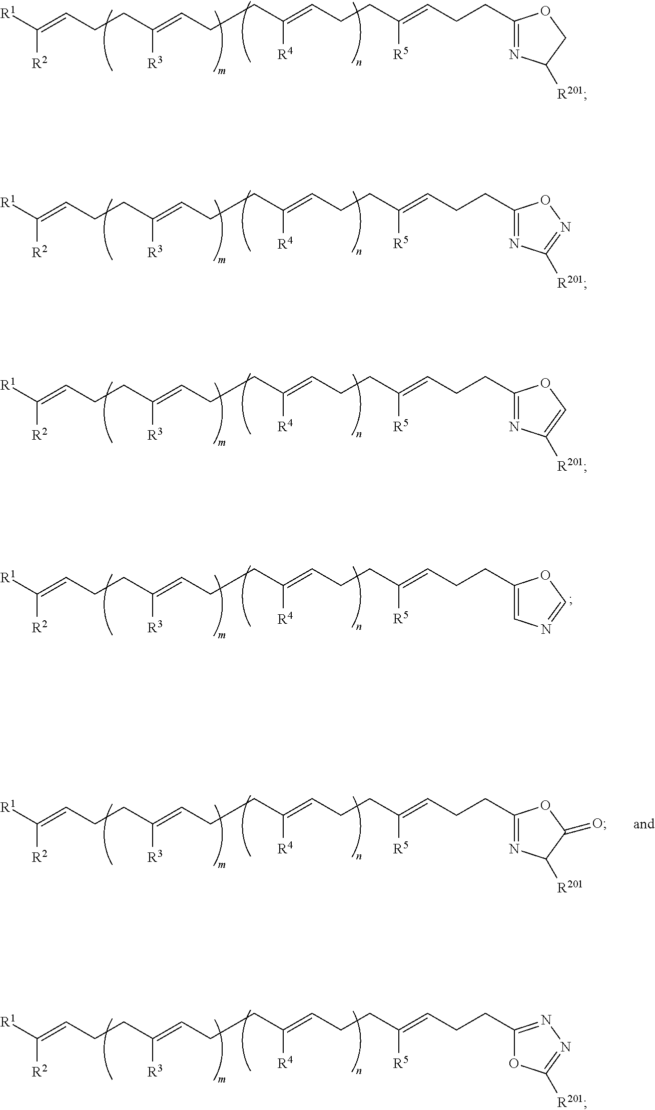
or pharmaceutically acceptable salt thereof, wherein
R²⁰¹ is selected from the group consisting of
hydrogen or hydroxy, oxo, —N(R⁴⁰)₂, C₁-C₆ alkoxy group, C₁-C₆ alkyl group, C₃-C₁₀ cycloalkyl, —CO₂H or an C₁-C₆ alkyl ester or an C₁-C₆ alkyl amide thereof, wherein the cycloalkyl group is optionally substituted with 1-3 C₁-C₆ alkyl groups,
a 5-9 membered heteroaryl or heterocyclyl containing up to 3 ring heteroatoms, wherein the heteroaryl or heterocyclyl is optionally substituted with 1-3 hydroxy, —N(R⁴⁰)₂, and C₁-C₆ alkyl group, and
benzyl or C₆-C₁₀ aryl, optionally substituted with 1-3 substituents selected from the group consisting of C₁-C₆ alkyl, C₁-C₆ alkoxy, hydroxy, and halo groups; and
the remaining variables are as defined in claim 1.

5. The compound of claim 4, wherein the compound has a Formula selected from the group consisting of: 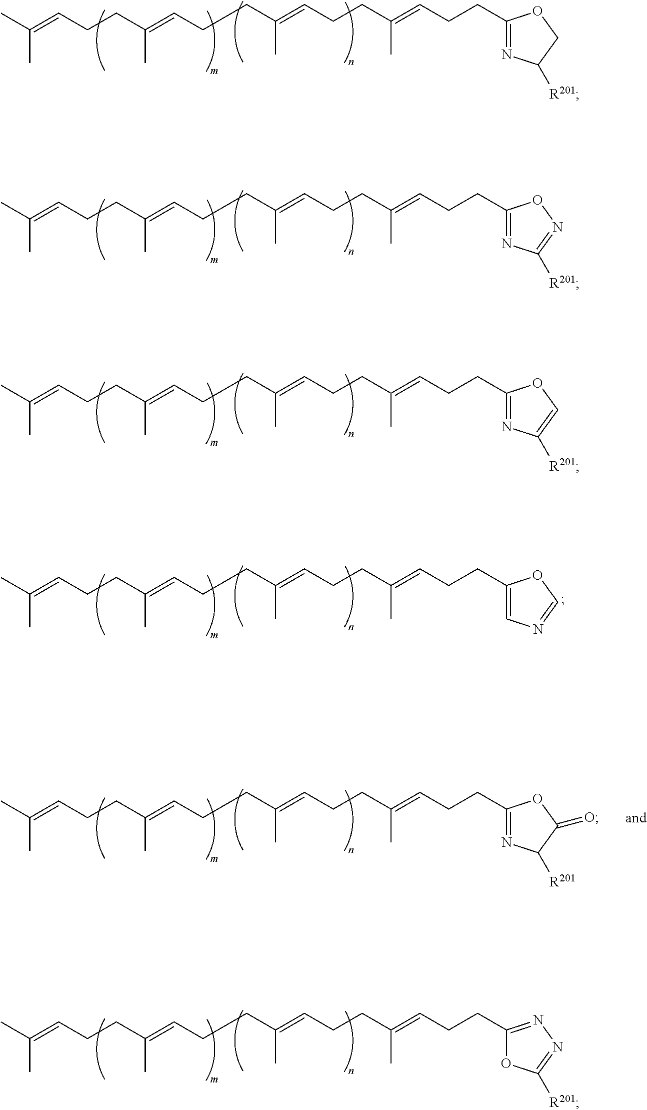
or pharmaceutically acceptable salt thereof.

6. The compound of claim 1, wherein the compound is of Formula (III): 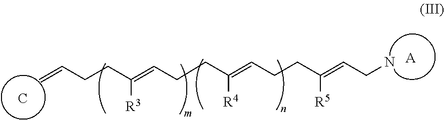
or pharmaceutically acceptable salt thereof, wherein
rings A and C are independently a 5-10 membered heteroaryl or heterocycle containing up to 6 ring heteroatoms, wherein the heteroatom is selected from the group consisting of O, N, S, and oxidized forms of N and S, and further wherein the heteroaryl, or heterocyclyl ring is optionally substituted with 1-3 substituents selected from the group consisting of:
hydroxy, oxo, —N(R⁴⁰)₂, C₁-C₆ alkoxy group, C₁-C₆ alkyl group, C₃-C₁₀ cycloalkyl, —CO₂H or an C₁-C₆ alkyl ester or an C₁-C₆ alkyl amide thereof, wherein the cycloalkyl group is optionally substituted with 1-3 C₁-C₆ alkyl groups,
a 5-9 membered heteroaryl or heterocyclyl containing up to 3 ring heteroatoms, wherein the heteroaryl or heterocyclyl is optionally substituted with 1-3 hydroxy, —N(R⁴⁰)₂, and C₁-C₆ alkyl group,
benzyl or C₆-C₁₀ aryl, optionally substituted with 1-3 substituents selected from the group consisting of C₁-C₆ alkyl, C₁-C₆ alkoxy, hydroxy, and halo groups; and
the remaining variables are as defined in claim 1.

7. The compound of claim 6, wherein the compound is of Formula (IIIa): 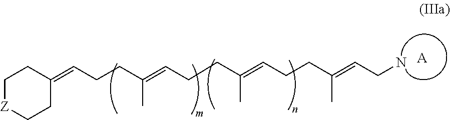
or pharmaceutically acceptable salt thereof;
Z is O or NR²¹⁰; and
R²¹⁰ is hydrogen or C₁-C₆ alkyl, CO₂H or an C₁-C₆ alkyl ester thereof; and
the remaining variables are as defined in claim 1.

8. The compound of claim 6, wherein the compound has a Formula selected from the group consisting of: 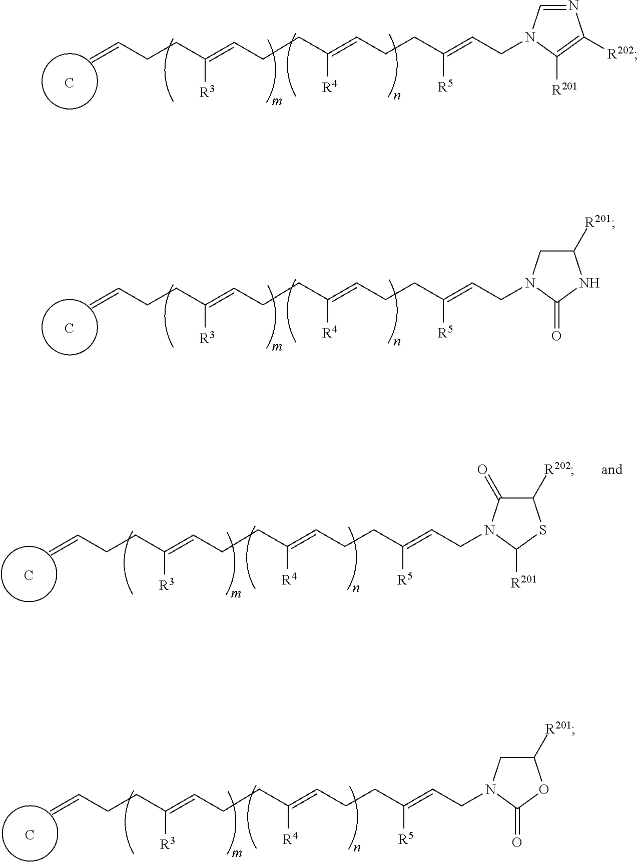
or pharmaceutically acceptable salt thereof, and
wherein R²⁰¹ and R²⁰² are independently hydrogen or hydroxy, oxo, —N(R⁴⁰)₂, C₁-C₆ alkoxy group, C₁-C₆ alkyl group, C₃-C₁₀ cycloalkyl, —CO₂H or an C₁-C₆ alkyl ester or an C₁-C₆ alkyl amide thereof, wherein the alkyl or cycloalkyl group is optionally substituted with 1-3 C₁-C₆ alkyl groups,
a 5-9 membered heteroaryl or heterocyclyl containing up to 3 ring heteroatoms, wherein the heteroaryl or heterocyclyl is optionally substituted with 1-3 hydroxy, —N(R⁴⁰)₂, and C₁-C₆ alkyl group,
benzyl or C₆-C₁₀ aryl, optionally substituted with 1-3 substituents selected from the group consisting of C₁-C₆ alkyl, C₁-C₆ alkoxy, hydroxy, and halo groups; and
the remaining variables are as defined in claim 1.

9. The compound of claim 8, wherein the compound has a Formula selected from the group consisting of: 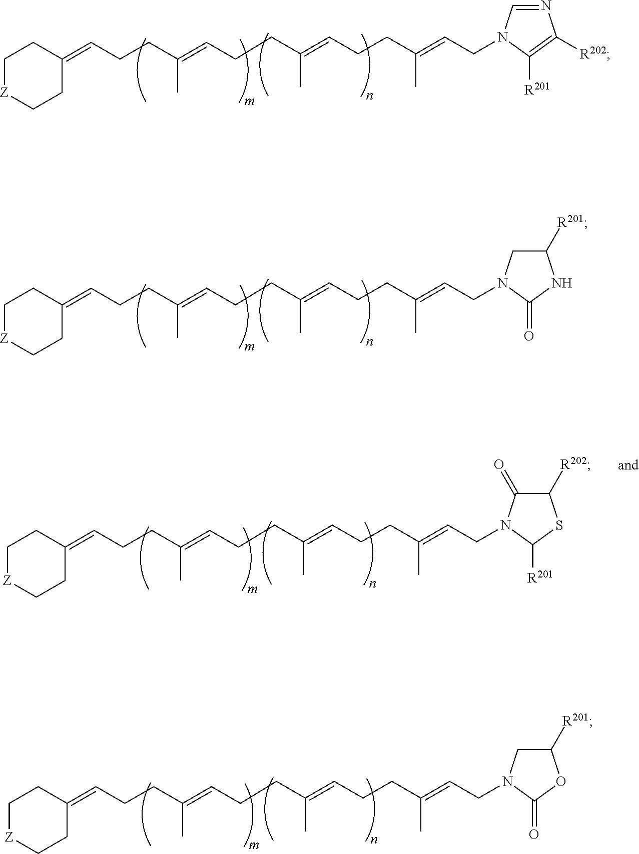
or pharmaceutically acceptable salt thereof;
Z is O or NR²¹⁰; and
R²¹⁰ is hydrogen or C₁-C₆ alkyl, CO₂H or an C₁-C₆ alkyl ester thereof and the remaining variables are as defined in claim 1.

10. The compound of claim 1, wherein the compound is of Formula (VI): 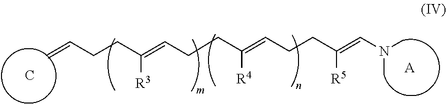
or pharmaceutically acceptable salt thereof, wherein
rings A and C are independently a 5-10 membered heteroaryl or heterocycle containing up to 6 ring heteroatoms, wherein the heteroatom is selected from the group consisting of O, N, S, and oxidized forms of N and S, and further wherein the heteroaryl, or heterocyclyl ring is optionally substituted with 1-3 substituents selected from the group consisting of:
hydroxy, oxo, —N(R⁴⁰)₂, C₁-C₆ alkoxy group, C₁-C₆ alkyl group, C₃-C₁₀ cycloalkyl, —CO₂H or an C₁-C₆ alkyl ester or an C₁-C₆ alkyl amide thereof, wherein the cycloalkyl group is optionally substituted with 1-3 C₁-C₆ alkyl groups,
a 5-9 membered heteroaryl or heterocyclyl containing up to 3 ring heteroatoms, wherein the heteroaryl or heterocyclyl is optionally substituted with 1-3 hydroxy, —N(R⁴⁰)₂, and C₁-C₆ alkyl group,
benzyl or C₆-C₁₀ aryl, optionally substituted with 1-3 substituents selected from the group consisting of C₁-C₆ alkyl, C₁-C₆ alkoxy, hydroxy, and halo groups; and
the remaining variables are as defined in claim 1.

11. The compound of claim 10, wherein the compound is of Formula (IVa): 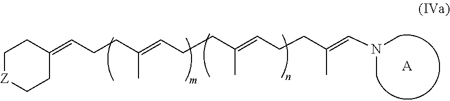
or pharmaceutically acceptable salt thereof;
Z is O or NR²¹⁰;
R²¹⁰ is hydrogen or C₁-C₆ alkyl, CO₂H or an C₁-C₆ alkyl ester thereof; and the remaining variables are as defined in claim 1.

12. The compound of claim 10, wherein the compound has a Formula selected from the group consisting of: 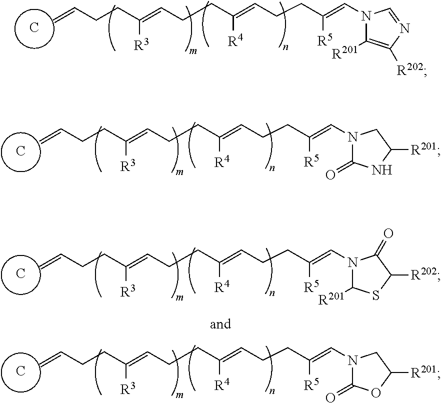
or pharmaceutically acceptable salt thereof,
wherein R²⁰¹ and R²⁰² are independently hydrogen or hydroxy, oxo, —N(R⁴⁰)₂, C₁-C₆ alkoxy group, C₁-C₆ alkyl group, C₃-C₁₀ cycloalkyl, —CO₂H or an C₁-C₆ alkyl ester or an C₁-C₆ alkyl amide thereof, wherein the alkyl or cycloalkyl group is optionally substituted with 1-3 C₁-C₆ alkyl groups,
a 5-9 membered heteroaryl or heterocyclyl containing up to 3 ring heteroatoms, wherein the heteroaryl or heterocyclyl is optionally substituted with 1-3 hydroxy, —N(R⁴⁰)₂, and C₁-C₆ alkyl group,
benzyl or C₆-C₁₀ aryl, optionally substituted with 1-3 substituents selected from the group consisting of C₁-C₆ alkyl, C₁-C₆ alkoxy, hydroxy, and halo groups; and
the remaining variables are as defined in claim 1.

13. The compound of claim 1, wherein the compound is of Formula (V): 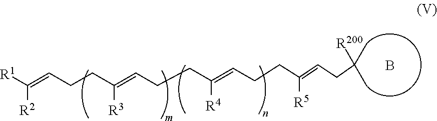
or pharmaceutically acceptable salt thereof;
R²⁰⁰ is hydrogen, CO₂H or an C₁-C₆ alkyl ester thereof; and the remaining variables are as defined in claim 1.

14. The compound of claim 1, wherein the compound is of Formula (Va): 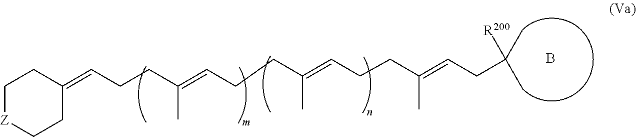
or pharmaceutically acceptable salt thereof;
Z is O or NR²¹⁰;
R²¹⁰ is hydrogen or C₁-C₆ alkyl, CO₂H or an C₁-C₆ alkyl ester thereof; and the remaining variables are as defined in claim 1.

15. The compound of claim 1, wherein the compound has a Formula selected from the group consisting of: 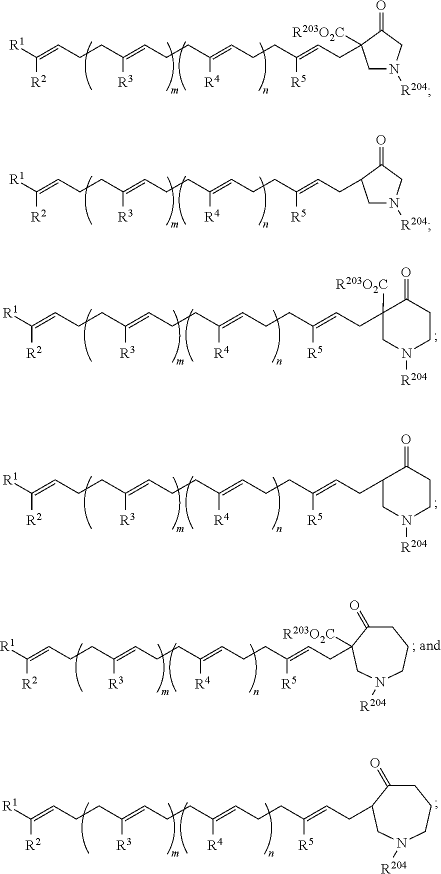
or pharmaceutically acceptable salt thereof, wherein
R²⁰³ is hydrogen or C₁-C₆ alkyl; and
R²⁰⁴ is hydrogen, —CO₂H or a C₁-C₆ alkyl ester or a C₁-C₆ alkyl amide thereof, —SO₂N(R⁴⁰)₂, C₁-C₆ alkyl, C₆-C₁₀ aryl, optionally substituted with 1-3 substituents selected from the group consisting of C₁-C₆ alkyl, C₁-C₆ alkoxy, hydroxy, and halo;
R¹ and R² are not C₁-C₆ alkyl; and R¹ and R² do not combine with the carbon to which they are attached to form a C₄-C₇ cycloalkyl ring; and
the remaining variables are as defined in claim 1.

16. The compound of claim 1, wherein the compound is of Formula (VI): 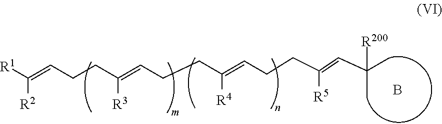
or pharmaceutically acceptable salt thereof;
R¹ and R² are not C₁-C₆ alkyl; and R¹ and R² do not combine with the carbon to which they are attached to form a C₄-C₇ cycloalkyl ring;
R²⁰⁰ is hydrogen, CO₂H or an C₁-C₆ alkyl ester thereof;
R¹ and R² are not C₁-C₆ alkyl; and R¹ and R² do not combine with the carbon to which they are attached to form a C₄-C₇ cycloalkyl ring; and
the remaining variables are as defined in claim 1.

17. The compound of claim 16, wherein the compound has a Formula selected from the group consisting of: 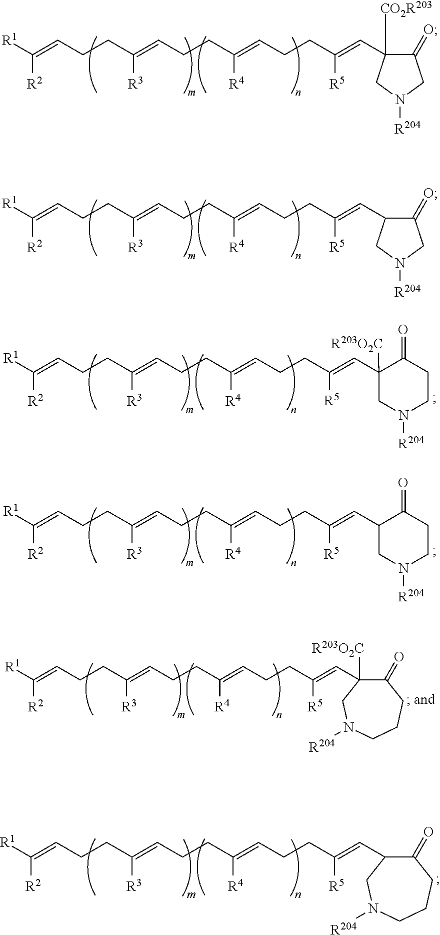
or pharmaceutically acceptable salt thereof, wherein
R²⁰³ is hydrogen or C₁-C₆ alkyl; and
R²⁰⁴ is hydrogen, —CO₂H or a C₁-C₆ alkyl ester or a C₁-C₆ alkyl amide thereof, —SO₂N(R⁴⁰)₂, C₁-C₆ alkyl, C₆-C₁₀ aryl, optionally substituted with 1-3 substituents selected from the group consisting of C₁-C₆ alkyl, C₁-C₆ alkoxy, hydroxy, and halo;
R¹ and R² are not C₁-C₆ alkyl; and R¹ and R² do not combine with the carbon to which they are attached to form a C₄-C₇ cycloalkyl ring; and
the remaining variables are as defined in claim 1.

18. The compound of claim 1, wherein the compound is of Formula (VII): 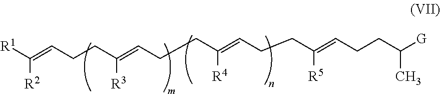
or pharmaceutically acceptable salt thereof.

19. The compound of claim 18, wherein the compound is of Formula (VIIa): 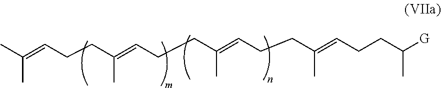
or pharmaceutically acceptable salt thereof.

20. The compound of claim 1, wherein the compound has a Formula selected from the group consisting of: 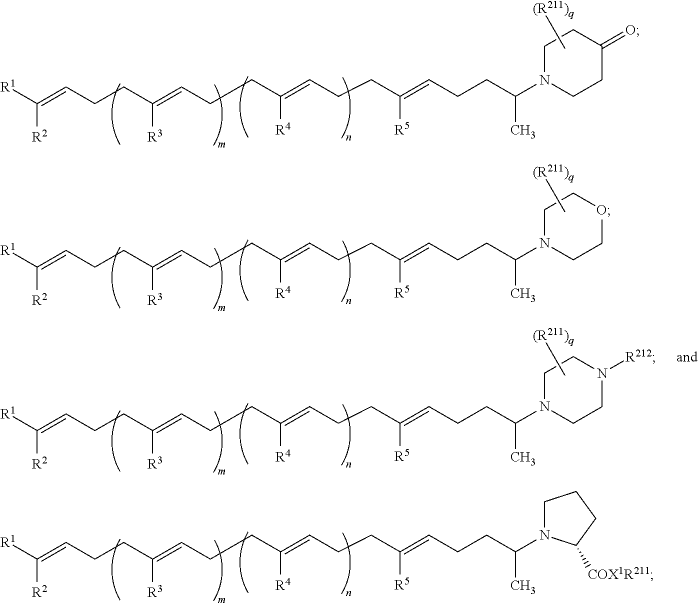
or pharmaceutically acceptable salt thereof; wherein
q is 0, 1, 2, 3 or 4;
X¹ is O or NR²¹⁴
R²¹¹ is selected from the group consisting of
hydrogen,
C₁-C₆ alkyl optionally substituted with 1-3 C₁-C₆ alkyl;
a 5-9 membered heteroaryl containing up to 3 ring heteroatoms, wherein the heteroaryl or heterocyclyl is optionally substituted with 1-3 hydroxy, —N(R⁴⁰)₂, and C₁-C₆ alkyl group, and
C₆-C₁₀ aryl, optionally substituted with 1-3 substituents selected from the group consisting of C₁-C₆ alkyl, C₁-C₆ alkoxy, hydroxy, and halo groups;
R²¹² is selected from the group consisting of
hydrogen, R²¹³, —CO—R²¹³ and —SO₂—R²¹³; wherein
R²¹³ is selected from the group consisting of:
C₁-C₆ alkyl optionally substituted with 1-3 C₁-C₆ alkyl;
a 5-9 membered heteroaryl containing up to 3 ring heteroatoms, wherein the heteroaryl is optionally substituted with 1-3 hydroxy, —N(R⁴⁰)₂, and C₁-C₆ alkyl group, and
C₆-C₁₀ aryl, optionally substituted with 1-3 substituents selected from the group consisting of C₁-C₆ alkyl, C₁-C₆ alkoxy, hydroxy, and halo groups; and
R²¹⁴ is hydrogen or C₁-C₆ alkyl optionally substituted with 1-3 C₁-C₆ alkyl; and
the remaining variables are as defined in claim 1.

21. A compound of Formula (VIII), (IX), (X), (XI) or (XII): 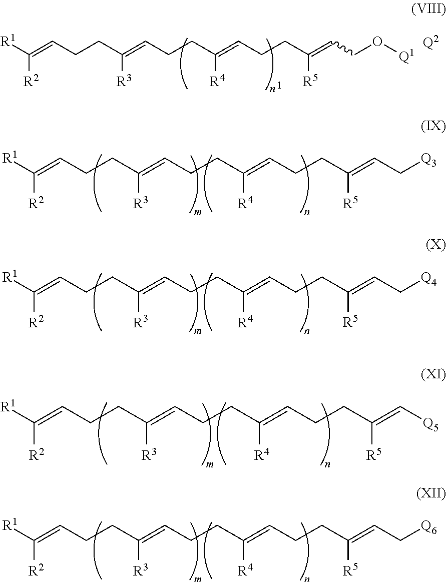
or pharmaceutically acceptable salt thereof, wherein
n¹ is 1 or 2;
n is 0, 1 or 2;
m is 0 or 1;
each R¹ and R² form, together with the carbon atom to which they are attached, a 5-6 membered heterocycle containing up to 3 ring heteroatoms, wherein the heteroatom is selected from the group consisting of O, N, S, and oxidized forms of N and S, and further wherein the heterocyclyl ring is optionally substituted with 1-3 substituents selected from the group consisting of:
hydroxy, oxo, —N(R⁴⁰)₂, C₁-C₆ alkoxy group, and C₁-C₆ alkyl group, wherein the alkyl group is optionally substituted with 1-3 substituents selected from hydroxy, NH₂, —CO₂H or an ester or an amide thereof, R⁴⁰ is defined as above,
each of R³, R⁴, and R⁵ independently are hydrogen or C₁-C₆ alkyl;
Q¹ is —(C═O)—, —(C═S)—, or —S(O₂)—;
Q² is hydrogen, R⁶, —O—R⁶, —NR⁷R⁸, or is a chiral moiety;
Q³ is —OH, —NR²²R²³—X—CO—NR²⁴R²⁵, —X—CS—NR²⁴R²⁵, or —X—SO₂—NR²⁴R²⁵;
Q⁴ is selected from the group consisting of: 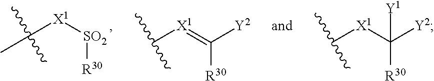
Q⁵ is —C(═O)H, —CO₂H or an ester or acyl halide thereof, wherein the ester is optionally substituted with —CO-phenyl; a 6-10 membered aryl or a 5-14 membered heteroaryl or heterocycle containing up to 6 ring heteroatoms, wherein the heteroatom is selected from the group consisting of O, N, S, and oxidized forms of N and S, and further wherein the aryl, heteroaryl, or heterocyclyl ring is optionally substituted with 1-3 substituents selected from the group consisting of:
hydroxy, oxo, —N(R⁴⁰)₂, C₁-C₆ alkoxy group, and C₁-C₆ alkyl group, wherein the alkyl group is optionally substituted with 1-3 substituents selected from hydroxy, NH₂, —CO₂H or an ester or an amide thereof,
a 5-9 membered heteroaryl containing up to 3 ring heteroatoms, wherein the heteroaryl is optionally substituted with 1-3 hydroxy, —N(R⁴⁰)₂, and C₁-C₆ alkyl group,
benzyl, and phenyl optionally substituted with 1-3 substituents selected from the group consisting of C₁-C₆ alkyl, C₁-C₆ alkoxy, hydroxy, and halo groups; and
when Q₅ is present:
R⁵ and Q₅ together with the intervening carbon atoms form a 6 membered aryl ring, or a 5-8 membered cycloalkenyl ring, or a 5-14 membered heteroaryl or heterocycle, wherein each aryl, cycloalkenyl, heteroaryl, or heterocycle, ring is optionally substituted with 1-2 substituents selected from the group consisting of halo, hydroxy, oxo, —N(R⁴⁰)₂, and C₁-C₆ alkyl;
Q⁶ is selected from the group consisting of: 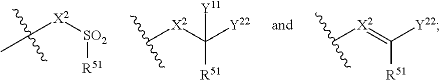
X is —O—, —S—, —NR²⁶—, or —CR²⁷R²⁸;
when X¹ is bonded via a single bond, X¹ is —O—, —NR³¹—, or —CR³²R³³—, and when X¹ is bonded via a double bond, X¹ is —CR³²—;
when X² is bonded via a single bond, X² is —O—, —NR⁵²—, or —CR⁵³R⁵⁴—, and when X² is bonded via a double bond, X² is —CR⁵³—;
Y¹ is hydrogen, —OH or —O—R¹⁰,
Y² is —OH, —OR¹¹ or —NHR¹², or Y¹ and Y² are joined to form an oxo group (═O), an imine group (═NR¹³), a oxime group (═N—OR¹⁴), or a substituted or unsubstituted vinylidene (═CR¹⁶R¹²);
Y^{1l} is hydrogen, —OH or —OR⁵⁵;
Y²² is —OH, —OR⁵⁶, —NHR⁵⁷, or —O—CO—NR⁵⁸R⁵⁹, or Y¹¹ and Y²² are joined to form an oxo group (═O), an imine group (═NR⁶⁰), a oxime group (═N—OR⁶¹), or a substituted or unsubstituted vinylidene (═CR⁶³R⁶⁴);
R⁶ is:
C₁-C₆ alkyl, optionally substituted with —CO₂H or an ester thereof, C₁-C₆ alkoxy, oxo, —OH, —CR═CR₂, —C≡CR, C₃-C₁₀ cycloalkyl, C₃-C₈ heterocyclyl, C₆-C₁₀ aryl, C₂-C₁₀heteroaryl, wherein each R independently is hydrogen or C₁-C₆ alkyl;
CO—C₁-C₆ alkyl;
C₃-C₁₀ cycloalkyl;
C₃-C₈ heterocyclyl;
C₆-C₁₀ aryl; or
C₂-C₁₀ heteroaryl;
wherein each cycloalkyl, heterocyclyl, aryl, or heteroaryl is optionally substituted with 1-3 alkyl groups; —CF₃, 1-3 halo, preferably, chloro or fluoro, groups; 1-3 nitro groups; 1-3 C₁-C₆ alkoxy groups; —CO-phenyl; or —NR¹⁸R¹⁹;
each R⁷ and R⁸ are independently hydrogen or defined as R⁶;
R¹⁰ is C₁-C₆ alkyl;
R¹¹ and R¹² are independently C₁-C₆ alkyl, C₃-C₁₀ cycloalkyl, —CO₂R¹⁵, or —CON(R¹⁵)₂, or R¹⁰ and R¹¹ together with the intervening carbon atom and oxygen atoms form a heterocycle optionally substituted with 1-3 C₁-C₆ alkyl groups;
R¹³ is C₁-C₆ alkyl or C₃-C₁₀ cycloalkyl optionally substituted with 1-3 C₁-C₆ alkyl groups;
R¹⁴ is hydrogen, C₃-C₈ heterocyclyl, or C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;
each R¹⁵ independently are hydrogen, C₃-C₁₀ cycloalkyl, C₁-C₆ alkyl optionally substituted with 1-3 substituents selected from the group consisting of —CO₂H or an ester thereof, aryl, or C₃-C₈ heterocyclyl, or two R¹⁵ groups together with the nitrogen atom they are bonded to form a 5-7 membered heterocycle;
R¹⁶ is hydrogen or C₁-C₆ alkyl;
R¹⁷ is hydrogen, C₁-C₆ alkyl substituted with 1-3 hydroxy groups, —CHO, or is CO₂H or an ester thereof;
each R¹⁸ and R¹⁹ independently is hydrogen; C₁-C₆ alkyl, optionally substituted with —CO₂H or an ester thereof, C₁-C₆ alkoxy, oxo, —CR═CR₂, —CCR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, or where R¹⁸ and R¹⁹ together with the nitrogen atom they are attached to form a 5-7 membered heterocycle;
each R²² and R²³ independently is hydrogen; C₁-C₆ alkyl, optionally substituted with C₁-C₆ alkoxy; and C₃-C₁₀ cycloalkyl;
each R²⁴ and R²⁵ independently is hydrogen; C₁-C₆ alkyl, optionally substituted with —CO₂H or an ester thereof, C₁-C₆ alkoxy, oxo, —CR═CR₂, —CCR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, or R²⁴ and R²⁵ together with the nitrogen atom they are attached to form a 5-7 membered heterocycle;
R²⁶ is hydrogen or together with R²⁴ or R²⁵ and the intervening atoms form a 5-7 membered heterocyclic ring optionally substituted with 1-3 C₁-C₆ alkyl groups;
each R²⁷ and R²⁸ independently are hydrogen, C₁-C₆ alkyl, —COR⁸¹ or —CO₂R⁸¹, or R²⁷ together with R²⁴ or R²⁵ and the intervening atoms form a 5-7 membered heterocyclyl ring optionally substituted with 1-3 C₁-C₆ alkyl groups;
R³⁰ is C₁-C₆ alkyl optionally substituted with 1-3 alkoxy or 1-5 halo group, C₂-C₆ alkenyl, C₂-C₆ alkynyl, C₃-C₁₀ cycloalkyl, C₆-C₁₀ aryl, C₃-C₈ heterocyclyl, or C₂-C₁₀ heteroaryl, wherein each cycloalkyl or heterocyclyl is optionally substituted with 1-3 C₁-C₆ alkyl groups, or wherein each aryl or heteroaryl is independently substituted with 1-3 C₁-C₆ alkyl or nitro groups, or R³⁰ is —NR³⁴R³⁵;
R³¹ is hydrogen or together with R³⁰ and the intervening atoms form a 5-7 membered ring optionally substituted with 1-3 C₁-C₆ alkyl groups;
each R³² and R³³ independently are hydrogen, C₁-C₆ alkyl, —COR⁸¹ or —CO₂R⁸¹, or R³² together with R³⁰ and the intervening atoms form a 5-7 membered cycloalkyl or heterocyclyl ring optionally substituted with oxo or 1-3 C₁-C₆ alkyl groups;
each R³⁴ and R³⁵ independently is hydrogen, C₁-C₆ alkyl, optionally substituted with —CO₂H or an ester thereof, C₃-C₁₀ cycloalkyl, C₃-C₈ heterocyclyl, C₆-C₁₀ aryl, or C₂-C₁₀ heteroaryl, or is C₃-C₁₀ cycloalkyl, C₃-C₈ heterocyclyl, C₆-C₁₀ aryl, or C₂-C₁₀ heteroaryl, wherein each cycloalkyl, heterocyclyl, aryl, or heteroaryl is optionally substituted with 1-3 alkyl groups, or R³⁴ and R³⁵ together with the nitrogen atom they are attached to form a 5-7 membered heterocycle;
each R⁴⁰ independently is hydrogen or C₁-C₆ alkyl;
R⁵¹ is C₁-C₆ alkyl, C₁-C₆ alkyl substituted with 1-3 alkoxy or 1-5 halo groups, C₂-C₆ alkenyl, C₂-C₆ alkynyl, C₃-C₁₀ cycloalkyl, C₃-C₈ heterocyclyl, C₆-C₁₀ aryl, C₂-C₁₀ heteroaryl, or —NR⁶⁵R⁶⁶, wherein each cycloalkyl or heterocyclyl is optionally substituted with 1-3 C₁-C₆ alkyl groups, and wherein each aryl or heteroaryl is optionally substituted independently with 1-3 nitro and C₁-C₆ alkyl groups;
R⁵² is hydrogen or together with R⁵¹ and the intervening atoms form a 5-7 membered ring optionally substituted with 1-3 C₁-C₆ alkyl groups;
each R⁵³ and R⁵⁴ independently are hydrogen, C₁-C₆ alkyl, —COR⁸¹, —CO₂R⁸¹, or —CONHR⁸², or R⁵³ together with R⁵¹ and the intervening atoms form a 5-7 membered cycloalkyl or heterocyclyl ring optionally substituted with 1-3 C₁-C₆ alkyl groups;
R⁵⁵ is C₁-C₆ alkyl;
each R⁵⁶ and R⁵⁷ independently are C₁-C₆ alkyl, C₃-C₁₀ cycloalkyl, —CO₂R⁶², or —CON(R⁶²)₂; or R⁵⁵ and R⁵⁶ together with the intervening carbon atom and oxygen atoms form a heterocycle optionally substituted with 1-3 C₁-C₆ alkyl groups;
R⁵⁸ is: C₃-C₁₀ cycloalkyl, C₁-C₆ alkyl optionally substituted with —OH, CO₂H or an ester thereof, or C₃-C₁₀ cycloalkyl, 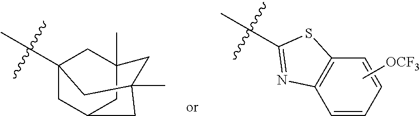
R⁵⁹ is hydrogen or C₁-C₆ alkyl;
R⁶⁰ is C₁-C₆ alkyl or C₃-C₁₀ cycloalkyl optionally substituted with 1-3 C₁-C₆ alkyl groups, or is: 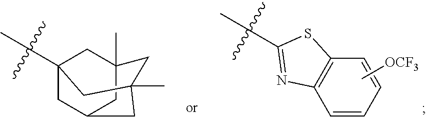
R⁶¹ is hydrogen, C₃-C₈ heterocyclyl, or C₁-C₆alkyl optionally substituted with a —CO₂H or an ester thereof or a C₆-C₁₀ aryl, C₂-C₆ alkenyl, C₂-C₆ alkynyl, C₃-C₁₀cycloalkyl, or a C₃-C₈ heterocyclyl, wherein each cycloalkyl, heterocyclyl, or aryl, is optionally substituted with 1-3 alkyl groups;
each R⁶² independently are hydrogen, C₃-C₁₀ cycloalkyl, C₁-C₆ alkyl optionally substituted with 1-3 substituents selected from the group consisting of —CO₂H or an ester thereof, aryl, C₃-C₈ heterocyclyl, or two R⁶² groups together with the nitrogen atom they are bonded to form a 5-7 membered heterocycle;
R⁶³ is hydrogen or C₁-C₆ alkyl;
R⁶⁴ is hydrogen, C₁-C₆ alkyl substituted with 1-3 hydroxy groups, —CHO, or is CO₂H or an ester thereof;
one or both of R⁶⁵ and R⁶⁶ independently are hydrogen, C₁-C₆ alkyl, optionally substituted with —CO₂H or an ester thereof, C₃-C₁₀ cycloalkyl, C₃-C₈ heterocyclyl, C₂-C₁₀ aryl, or C₂-C₁₀ heteroaryl, or is C₃-C₁₀ cycloalkyl, C₃-C₈ heterocyclyl, C₆-C₁₀aryl, or C₂-C₁₀ heteroaryl, wherein each cycloalkyl, heterocyclyl, aryl, or heteroaryl is optionally substituted with 1-3 alkyl groups, or R⁶⁵ and R⁶⁶ together with the nitrogen atom they are bonded to form a 5-7 membered heterocycle, and if only one of R⁶⁵ and R⁶⁶ are defined as above, then the other one is 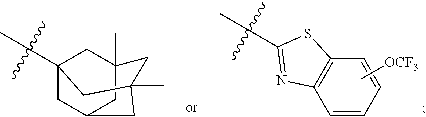
R⁸¹ is C₁-C₆ alkyl; and
R⁸² is:

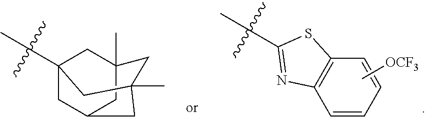

22. The compound of claim 21, wherein R¹ and R² are together with the carbon atom they are attached to form 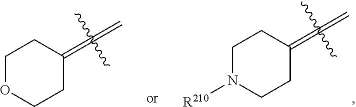
R²¹⁰ is hydrogen or C₁-C₆ alkyl, CO₂H or an C₁-C₆ alkyl ester thereof.

23. The compound of claim 21, wherein R³, R⁴ and R⁵ are methyl.

24. A pharmaceutical composition comprising a compound of any one of claims 1-23 and a pharmaceutically acceptable excipient.
